(19) United States
(12) Patent Application Publication
(10) Pub. No.: US 2002/0088015 A1
(43) Pub. Date: Jul. 4, 2002

(54) WILMS' TUMOR WT1 BINDING PROTEINS

(76) Inventor: YANG SHI, BROOKLINE, MA (US)

Correspondence Address: BANNER & WITCOFF, LTD., 28 STATE STREET, 28th FLOOR, BOSTON, MA, 02109, US

(21) Appl. No.: 09/269,390

(22) PCT Filed: Sep. 26, 1997
(86) PCT No.: PCT/US97/17382

(51) Int. Cl. A01K 67/00; A01K 67/033; C07H 21/04; C12N 1/20; C12N 15/00; C12N 15/09; C12N 15/63; C12N 15/70; C12N 15/74; C12N 5/00; C12N 5/02; C07K 1/00; C07K 14/00; C07K 17/00; C07K 16/00; C08H 1/00; C12Q 1/68; G01N 33/53

(52) U.S. Cl. 800/13; 530/350; 530/403; 530/387.1; 536/23.4; 536/23.5; 435/320.1; 435/252.3; 435/325; 435/7.1; 435/6

(57) ABSTRACT

The present invention comprises compositions and methods that relate to diagnostic and therapeutic assays and reagents for detecting and treating disorders involving aberrant assembly of WT1 complexes. Moreover, drug discovery assays are provided for identifying agents which can modulate the binding of one or more of the subject WT1-binding proteins with WT1 or other transcriptional regulatory proteins. Such agents can be useful therapeutically to alter the growth and/or differentiation of a cell, but can also be used in vitro as cell-culture additives for controlling proliferation and/or differentiation of cultured cells and tissue.

### BACKGROUND OF THE INVENTION

Wilm's tumor is a pediatric nephroblastoma that is one of the most frequent solid tumors in children, occurring in about one in ten thousand live births. The tumor appears to arise from the embryonal metanephric mesenchyme during differentiative events in the embryonic kidney and is often composed of stromal, epithelial and mesenchymal cells in a disorganized array. Abortive glomeruli and tubular elements are present, indicating a block in proper nephrogenesis. Treatment of this malignant disorder includes surgery and chemotherapy for all patients and radiation therapy for those with advanced disease or specific adverse prognostic features.

Wilm's tumors (WT) are associated with certain congenital defects, in particular, sporadic aniridia (a malformation of the iris and surrounding tissue), hemihypertrophy, Beckwith-Wiedemann Syndrome (BWS, a congenital overgrowth syndrome characterized by growth abnormalities and a predisposition to several embryonal neoplasms, including WT), Denys Drash Syndrome (DDS, which consists of the triad WT, intersex disorders, and nephropathy), and various anomalies of the genitourinary tract. In male patients, aniridia and WT are often associated with a genitourinary malformations and mental retardation, giving rise to the WAGR Syndrome.

Wilm's tumor has been linked to inactivation of the tumor-suppressor gene “WT1” at the 11p13 chromosomal locus (Haber et al. (1992) Adv Cancer Res 59:41-68). WT1 encodes a developmentally regulated transcription factor of 52-54 kDa. The Wilms tumor candidate gene WT1 is a tumor suppressor gene expressed in the developing kidney and in the adult urogenital system (reviewed in Haber, D. A. et al. (1992) New Biol. 4:97-106; Rauscher, F. J. (1993) FASEB J. 7:896-903; Reddy, J. C. et al. (1996) Biochimica et Biophysica Acta 1287:1-28). The WT1 gene is deleted or mutated in approximately 10% of sporadic Wilms tumors and in nearly 100% of Denys-Drash patients (reviewed in Dignam, J. D. et al. (1983) Nucleic Acids Res 11:1475-1489; Haber, D. A. et al. (1992) Adv. Cancer. Res. 59:41-68). WT1 protein has been shown to suppress cell growth in both Wilms tumor and non-Wilms' tumor cells (Haber, D. A. et al. (1993) Science 262:2057-2059; Kudoh, T. et al. (1995) Proc. Natl. Acad. Sci USA 92:4517-4521; Luo, X. -N. et al. (1995) Oncogene 11:734-750). The C-terminus contains four zinc fingers which confer binding specificity to the EGR1 DNA consensus (Rauscher et al. (1990) Science 1259-1262), while the N-terminus mediates transcriptional repression in transient transfection assays (Madden et al. (1991) Science 253:1550-1553; and Drummond et al. (1992) Science 257:674-678). A pattern of alternative splicing leads to distinct WT1 gene products (Haber et al. (1991) PNAS 88:9618-9622), and cells expressing WT1 produce four WT1 mRNAs reflecting different combinations of the alternative exons.

WT1 shows an expression pattern restricted both temporally and spatially during development. During embryonic development, WT1 transcripts are found in the kidney where the condensing metanephric mesenchyme and primitive renal vesicle are formed, the gonadal ridge mesothelia, and the mesothelial lining of the coelomic cavity and the organs it contains (Sharma et al. (1992) Cancer Research 52:6407-6412; and Jones et al. (1990) Nature 346:194-197). Transcripts are also found in the spleen, which is consistent with WT1 expression found in hematopoietic cell lines. Surprisingly, strong WT1 expression is also observed in the spinal cord ventral horn motor neurons and in the area posterior of the rat brain (Sharma et al. supra). The latter pattern of expression in the central nervous system persists in the adult animal.

WT1 has been proposed to exert its effects on cell proliferation or differentiation through specifically repressing or activating other cellular genes. The expression pattern of WT1 observed in a number of species supports its role as a tumor suppressor gene in kidney, and extends its possible functions to differentiation events in other organs. Moreover, WT1 acts as a transcriptional suppressor of growth-related genes, including the PDGF A-chain (Wang et al. (1992) J Biol Chem 267:21999-22002), IGF II (Drummond et al., supra), and EGR-1 genes (Madden et al. (1991) Science 253:1550-1553), supporting the potential of WT1 as a tumor suppressor. Classification of WT1 as a tumor suppressor gene is based on detection in tumor specimens of mutations within genes that inactivate the protein, such as small deletions and point mutations in the zinc fingers of WT1 that abolish DNA binding in a number of Wilm's tumors (Little et al. (1992) PNAS 89:4791-4795). Functional loss of WT1 can result in the unregulated synthesis of growth factors such as the PDGF A-chain and IGF-II in kidney blastemal cells.

The four alternatively spliced WT1 mRNA isoforms (A, B, C, D) encode zinc finger-containing proteins of 52-54 kD (Haber, D. A. et al. (1991) Proc. Natl. Acad. Sci. USA 88:9618-9622). Compared with isoform A, isoforms B and D contain 17 additional amino acids encoded by exon 5, inserted between the transactivation and DNA binding domains. Isoforms C and D contain an additional 9 nucleotides encoding lysine, threonine and serine residues inserted between zinc fingers 3 and 4 (Haber, D. A. et al. (1991) Proc. Natl. Acad. Sci. USA 88:9618-9622). Different isoforms of WT1 have been suggested to play different biological roles (Englert, C. et al. (1995) Proc. Natl. Acad Sci USA 92:11960-11964; Larsson, S. H. et al. (1995) Cell 81:391-401). The A and B isoforms of WT1 have been shown to bind the same consensus sequence that constitutes the recognition site for the Early Growth Response (EGR) family of transcription factors (Rauscher III, F. et al. (1990) Science 250:1259-1263). Isoforms C and D recognize related but distinct DNA sequences (Drummond, I. A. et al. (1994) Mol. Cell. Biol. 14:3800-3809; Rauscher III, F. et al. (1990) Science 250:1259-1263; Wang, Z. Y. et al. (1995) Oncogene 10:415422), suggesting that they may regulate different sets of genes.

WT1 like many DNA-binding transcription factors is capable both of repression and activation of transcription, according to the promoter and the physiological context (Madden, S. L. et al. (1991) Science 253:1550-1553; Reddy, J. C. et al. (1995) J. Biol. Chem. 270: 10878-10884; Wang, Z. -Y. et al. (1993) J. Bio. Chem. 268:9972-9975). WT1 negatively regulates many growth-related genes (Drummond, I. A. et al. (1992) Science 257:674-678; Gashler, A. L. et al. (1992) Proc. Natl. Acad Sci. USA 89:10984-10988; Madden, S. L. et al. (1991) Science 253:1550-1553; Wang, Z. Y. et al. (1992) J. Biol. Chem. 267:21999-22002; Werner, H. et al. (1993) Proc. Natl. Acad. Sci. USA 90:5828-5832), some of which may be physiologically relevant target genes. Mutated WT1 transcripts in Wilms' tumor samples encode mutant proteins that are defective for transcriptional repression activity (Haber, D. A. et al. (1993) Science 262:2057-2059; Park, S. et al. (1993) Cancer Res. 53:4757-4760). Heterozygous WT1 mutations are also associated with the disease. These WT1 mutants were shown to function as dominant negatives that inhibit the transcriptional activation and repression functions of the wild-type WT1 allele (Moffett, P. et al. (1995) Proc. Natl. Acad. Sci. USA. 92:11105-11109; Reddy, J. C. et al. (1995) J. Biol. Chem. 270: 10878-10884). The loss of WT1 transcriptional functions and/or an imbalance in its transcriptional repression and activation activities may lead to deregulated cell growth which contributes to tumorigenesis.

WT1 can physically interact with p53 (Maheswaran et al. (1993), PNAS 90:5100-5104) and this interaction modulates the ability of each protein to transactivate their respective targets. In fact, in contrast to the proposed function of WT1 as a transcriptional repressor, potent transcriptional activation by WT1 of reporter genes driven by EGR1 in cells lacking wild type p53 indicates that transcriptional repression is not an intrinsic property of WT1. Instead, transcriptional repression by WT1 may result from its interaction with p53.

This invention addresses one of the central questions regarding the mechanisms that control WT1 dual transcriptional activity. One model postulates that cellular proteins that interact with WT1 can influence its activity. The WT1/p53 interaction leads to the inhibition of p53-mediated apoptosis (Maheswaren, S. et al. (1995) Genes & Dev. 9:2143-2156). Genes other than p53 can modulate the transcriptional activities of WT1. Null mutations of WT1 in homozygous mice are embryonic lethal due to the failure in heart and kidney development (Kreidberg, J. A. et al. (1993) Cell 74:679-691), while most of the mice null for p53 are morphologically normal (Deluca, N. A. et al. (1992) Nature 56:215-221). In addition, analyses of Wilms' tumor samples revealed that the majority of Wilms tumors do not contain p53 mutations (Malkin, D. et al. (1994) Cancer Res. 54:2077-2079), although p53 defects are associated with anaplastic Wilms' tumors (Bardeesy, N. et al. (1994) Nature Genetics 7:91-96). To systematically identify proteins that interact with WT1, and to determine the functional consequences of such interactions, WT1-interacting proteins and the regulatory consequences of interaction with WT1 are here isolated and their effects on WT1 are described.

Unless otherwise defined, all technical and scientific terms used herein have the same meaning as commonly understood by one of ordinary skill in the art to which this invention belongs. Although methods and materials similar or equivalent to those herein can be used in the practice or testing of the present invention, the preferred methods and materials are described below. All publications, patent applications, patents, and other references mentioned herein are incorporated by reference. In addition, the materials, methods, and examples are illustrative only and not intended to be limiting.

### SUMMARY OF THE INVENTION

The present invention relates to the discovery in eukaryotic cells, particularly human cells, of novel protein-protein interactions between the Wilms tumor regulatory protein WT1 and certain cellular proteins, referred to hereinafter as “WT1-binding proteins” or “WT1-BP”.

In general, the invention features a WT1-BP^{Ciao-1} polypeptide, preferably a substantially pure preparation of a WT1-BP^{Ciao-1} polypeptide, or a recombinant WT1-BP^{Ciao-1} polypeptide. In preferred embodiments the polypeptide has a biological activity associated with its binding to WT1, e.g., it retains the ability to bind to a WT1 protein, though it may be able to either agonize or antagonize assembly of WT1-containing transcriptional protein complexes. The polypeptide can be identical to the Ciao-1 polypeptide shown in SEQ ID No: 3, or it can be homologous to that sequence. For instance, the Ciao-1 polypeptide preferably has an amino acid sequence at least 60% homologous or identical to the amino acid sequence in SEQ ID No: 3, though higher sequence homologies or identities of, for example, 80%, 90% or 95% are also contemplated.

The Ciao-1 polypeptide can comprise the full length protein represented in SEQ ID No: 3, or it can comprise a fragment of that protein, which fragment can be, for instance, at least 5, 10, 20, 50 or 100 amino acids in length. As described below, the Ciao-1 polypeptide can be either an agonist (e.g. mimics), or alternatively, an antagonist of a biological activity of a naturally occuring form of the Ciao-1 protein, e.g., the Ciao-1 polypeptide is able to modulate WT1-mediated gene expression in at least one tissue in which the WT1 protein is expressed, such as in urogenital tissue (such as bladder, gonadal tissues—i.e. ovarian or testicular), in renal tissue, in mesothelium, in hematopoietic cells, or in neural tissue.

In a preferred embodiment, a peptide having at least one biological activity of the subject Ciao-1 polypepide may differ in amino acid sequence from the sequence in SEQ ID No: 3, but such differences result in a modified protein which functions in the same or similar manner as the native WT1-binding protein or which has the same or similar characteristics of the native WT1-binding protein. However, homologs of the naturally occuring Ciao-1 protein are contemplated which are antagonistic of the normal cellular role of the naturally occurring Ciao-1 protein. For example, the homolog may be capable of interfering with the ability of WT1 to modulate gene expression, e.g. of developmentally or growth regulated genes.

In yet other preferred embodiments, a WT1-binding protein is a recombinant fusion protein which includes a second polypeptide portion, e.g., a second polypeptide having an amino acid sequence unrelated to WT1-BPCiao-1, e.g. the second polypeptide portion is glutathione-S-transferase, e.g. the second polypeptide portion is a DNA binding domain of transcriptional regulatory protein, e.g. the second polypeptide portion is an RNA polymerase activating domain, e.g. the fusion protein is functional in a two-hybrid assay.

Yet another aspect of the present invention concerns an immunogen comprising a Wt1-binding protein, for example a WT1-BP^{Ciao-1} peptide in an immunogenic preparation, the immunogen being capable of eliciting an immune response specific for said WT1-BP polypeptide; e.g. a humoral response, e.g. an antibody response: e.g. a cellular response. In preferred embodiments, the immunogen comprises an antigenic determinant, e.g. a unique determinant, from a protein represented by SEQ ID No: 3.

A still further aspect of the present invention features an antibody preparation specifically reactive with an epitope of the WT1-BP^{Ciao-1} immunogen.

In another aspect, the invention features a WT1-6BP^{par-4} polypeptide, preferably a substantially pure preparation of a WT-1-BP^{par-4} polypeptide, or a recombinant WT1-BP^{par-4} polypeptide. As above, in preferred embodiments the par-4 polypeptide has a biological activity associated with its binding to WT1, e.g., it retains the ability to bind to a WT1 protein, and can either agonize or antagonize assembly of WT1-containing transcriptional protein complexes. The polypeptide can be identical to the par-4 polypeptide shown in SEQ ID No: 4, or can comprise a polypeptide fragment homologous to that sequence. For instance, the par-4 polypeptide preferably has an amino acid sequence at least 60% homologous or identical to the amino acid sequence in SEQ ID No: 4, though higher sequence homologies or identities of, for example, 80%, 90% or 95% are also contemplated. The par-4 polypeptide can comprise the full length protein represented in SEQ ID No: 4, or it can comprise a fragment of that protein, which fragment can be, for instance, at least 5, 10, 20, 50 or 100 amino acids in length. The par-4 polypeptide can be either an agonist (e.g. mimics), or alternatively, an antagonist of a biological activity of a naturally occuring form of the par-4 protein, e.g., the par-4 polypeptide is able to modulate WT1-mediated gene expression in at least one tissue in which the WT1 protein is expressed, such as in urogenital tissue (such as bladder, gonadal tissues, e.g., ovarian or testicular), in renal tissue, in mesothelium, in hematopoietic cells, or in neural tissue.

In a preferred embodiment, a peptide having at least one biological activity of the subject WT1-BP^{par-4} polypeptide may differ in amino acid sequence from the sequence in SEQ ID No: 4, but such differences result in a modified protein which functions in the same or similar manner as the native WT1-binding protein or which has the same or similar characteristics of the native WT1-binding protein. However, homologs of the naturally occuring par-4 protein are contemplated which are antagonistic of the normal cellular role of the naturally occurring par-4 protein. For example, the homolog may be capable of interfering with the ability of WT1 to modulate gene expression, e.g. of developmentally or growth regulated genes.

In yet other preferred embodiments, the par-4 protein is a recombinant fusion protein which includes a second polypeptide portion, e.g., a second polypeptide having an amino acid sequence unrelated to WT-1-BP^{par-4} protein, e.g. the second polypeptide portion is glutathione-S-transferase, e.g. the second polypeptide portion is a DNA binding domain of transcriptional regulatory protein, e.g. the second polypeptide portion is an RNA polymerase activating domain, e.g. the fusion protein is functional in a two-hybrid assay.

Yet another aspect of the present invention concerns an immunogen comprising a par-4 polypeptide in an immunogenic preparation, the immunogen being capable of eliciting an immune response specific for said polypeptide; e.g. a humoral response, e.g. an antibody response; e.g. a cellular response. In preferred embodiments, the immunogen comprises an antigenic determinant, e.g. a unique determinant, from a protein represented by SEQ ID No: 4.

A still further aspect of the present invention features an antibody preparation specifically reactive with an epitope of the WT1-BP^{par-4} immunogen.

Another aspect of the present invention provides a substantially isolated nucleic acid having a nucleotide sequence which encodes a WT-1-BP^{Ciao-1} polypeptide. In preferred embodiments: the encoded polypeptide specifically binds a WT1 protein and is able to agonize or antagonize assembly of WT1-containing transcriptional protein complexes. The coding sequence of the nucleic acid can comprise a Ciao-1-encoding sequence which can be identical to the Ciao-1 cDNA shown in SEQ ID No: 1, or it can merely be homologous to that sequence. For instance, the Ciao-1-encoding sequence preferably has a sequence at least 60% homologous to the nucleotide sequence in SEQ ID No: 1, though higher sequence homologies of, for example, 80%, 90% or 95% are also contemplated. The Ciao-1 polypeptide encoded by the nucleic acid can comprise the nucleotide sequence represented in SEQ ID No: 1 which encodes the full length protein, or it can comprise a fragment of that nucleic acid, which fragment may be, for instance, encode a fragment of Ciao-1 which is, for example, at least 5, 10, 20, 50 or 100 amino acids in length. The Ciao-1 polypeptide encoded by the nucleic acid can be either an agonist (e.g. mimics), or alternatively, an antagonist of a biological activity of a naturally occuring form of the Ciao-1 protein, e.g., the Ciao-1 polypeptide is able to modulate WT1-mediated gene expression in at least one tissue in which the WT1 protein is expressed, such as in urogenital tissue (such as bladder, gonadal tissues—i.e. ovarian or testicular), in renal tissue, in mesothelium, in hematopoietic cells, or in neural tissue.

Furthermore, in certain preferred embodiments, the subject WT1-BP^{Ciao-1} nucleic acid will include a transcriptional regulatory sequence, e.g. at least one of a transcriptional promoter or transcriptional enhancer sequence, which regulatory sequence is operably linked to the WT-1-BP^{Ciao-1} gene sequence. Such regulatory sequences can be used in to render the WT1-BP^{Ciao-1} gene sequence suitable for use as an expression vector.

In yet a further preferred embodiment, the nucleic acid hybridizes under stringent conditions to a nucleic acid probe corresponding to at least 12 consecutive nucleotides of SEQ ID No: 1; preferably to at least 20 consecutive nucleotides of SEQ ID No: 1; more preferably to at least 40 consecutive nucleotides of SEQ ID No: 1.

Another aspect of the present invention provides a substantially isolated nucleic acid having a nucleotide sequence which encodes a WT-1-BP^{par-4} polypeptide. In preferred embodiments: the encoded polypeptide specifically binds a WT1 protein and is able to agonize or antagonize assembly of WT1-containing transcriptional protein complexes. The coding sequence of the nucleic acid can comprise a par-4-encoding sequence which can be identical to the par-4 cDNA shown in SEQ ID No: 2, or it can merely be homologous to that sequence. For instance, the par-4-encoding sequence preferably has a sequence at least 60% homologous to the nucleotide sequence in SEQ ID No: 2, though higher sequence homologies of, for example, 80%, 90% or 95% are also contemplated. The par-4 polypeptide encoded by the nucleic acid can comprise the nucleotide sequence represented in SEQ ID No: 2 which encodes the full length protein, or it can comprise a fragment of that nucleic acid, which fragment may be, for instance, encode a fragment of par-4 which is, for example, at least 5, 10, 20, 50 or 100 amino acids in length. The par-4 polypeptide encoded by the nucleic acid can be either an agonist (e.g. mimics), or alternatively, an antagonist of a biological activity of a naturally occuring form of the par-4 protein, e.g., the par-4 polypeptide is able to modulate WT1-mediated gene expression in at least one tissue in which the WT1 protein is expressed, such as in urogenital tissue (such as bladder, gonadal tissues—i.e. ovarian or testicular), in renal tissue, in mesothelium, in hematopoietic cells, or in neural tissue.

Furthermore, in certain preferred embodiments, the subject WT-1-BP^{par-4} nucleic acid will include a transcriptional regulatory sequence, e.g. at least one of a transcriptional promoter or transcriptional enhancer sequence, which regulatory sequence is operably linked to the WT1-BP^{par-4} gene sequence. Such regulatory sequences can be used in to render the WT-1-BP^{par-4} gene sequence suitable for use as an expression vector.

In yet a further preferred embodiment, the nucleic acid hybridizes under stringent conditions to a nucleic acid probe corresponding to at least 12 consecutive nucleotides of SEQ ID No: 2; preferably to at least 20 consecutive nucleotides of SEQ ID No: 2; more preferably to at least 40 consecutive nucleotides of SEQ ID No: 2.

The invention also features transgenic non-human subject animals, e.g. mice, rats, rabbits or pigs, having a transgene, e.g., animals which include (and preferably express) a heterologous form of one of the WT1-BP genes described herein, e.g. a gene derived from humans, or which misexpress an endogenous WT1-BP gene, e.g., an animal in which expression of one or more of the subject WT-1-binding proteins is disrupted. Such a transgenic animal can serve as an animal model for studying cellular disorders comprising mutated or mis-expressed WT1-BP alleles or for use in drug screening.

The invention also provides a probe/primer comprising a substantially purified oligonucleotide, wherein the oligonucleotide comprises a region of nucleotide sequence which hybridizes under stringent conditions to at least 10 consecutive nucleotides of sense or antisense sequence of one of SEQ ID Nos: 1-2, or naturally occurring mutants thereof. In preferred embodiments, the probe/primer further includes a label group attached thereto and able to be detected. The label group can be selected, e.g., from a group consisting of radioisotopes, fluorescent compounds, enzymes, and enzyme co-factors. Probes of the invention can be used as a part of a diagnostic test kit for identifying transformed cells, such as for detecting in a sample of cells isolated from a patient, a level of a nucleic acid encoding one of the subject WT-1-binding proteins; e.g. measuring the WT1-BP mRNA level in a cell, or determining whether the genomic WT1-BP gene has been mutated or deleted. Preferably, the oligonucleotide is at least 10 nucleotides in length, though primers of 20, 30, 50, 100, or 150 nucleotides in length are also contemplated.

In yet another aspect, the invention provides an assay for screening test compounds for an agent such as an inhibitor, or alternatively, a potentiator, of an interaction between a WT1-binding protein and a WT1 protein. An exemplary method includes the steps of (i) combining a viral WT1 protein, an WT1-BP, e.g., a WT1-BP of the invention (e.g. a protein expressed from one of the clones selected from the group Ciao-1, par-4. and homologs, and a test compound, e.g., under conditions wherein, but for the test presence of the agent or compound, the WT1 protein and the WT1-binding protein are able to interact; and (ii) detecting the formation of a complex which includes the WT1 protein and the WT1-binding protein. A statistically significant change, such as a decrease, in the formation of the complex in the presence of a test compound (relative to what is seen in the absence of the test compound) is indicative of a modulation, e.g., inhibition, of the interaction between WT1 and the WT1-binding protein. In preferred embodiments: the WT1 protein is a Wilm's tumor WT1 protein. Moreover, primary screens are provided in which the WT1 protein and the WT1-binding protein are combined in a cell-free system and contacted with the test compound; i.e. the cell-free system is selected from a group consisting of a cell lysate and a reconstituted protein mixture. Alternatively, WT1 and the WT1-binding protein are simultaneously expressed in a cell, and the cell is contacted with the test compound, e.g. as an interaction trap assay (two hybrid assay).

The present invention also provides a method for treating a subject such as a human or an animal, having unwanted cell growth characterized by a loss of wild-type function of one or more of the subject WT1-binding proteins, comprising administering a therapeutically effective amount of an agent able to inhibit the interaction of the WT1-binding protein with other cellular or viral proteins for example, interaction with WT1. In one embodiment, the method comprises administering a nucleic acid construct encoding a polypeptides represented in one of SEQ ID Nos: 3 and 4, under conditions wherein the construct is incorporated by cells deficient in that WT1-binding protein, and under conditions wherein the recombinant gene is expressed, e.g. by gene therapy techniques. In other embodiments, the action of a naturally-occurring WT-1-binding protein is antagonized by therapeutic expression of a WT1-BP homolog which is an antagonist of, for example, assembly of functional WT1 transcriptional regulatory complexes, or by delivery of an antisense nucleic acid molecule which inhibits transcription and/or translation of the targeted WT1-BP gene.

Another aspect of the present invention provides a method of determining if a subject, e.g. a human patient, is at risk for a disorder characterized by unwanted cell proliferation. The method includes detecting, in a tissue of the subject, the presence or absence of a genetic lesion characterized by at least one of (i) a mutation of a gene encoding a protein represented by one of SEQ ID Nos: 3 and 4, or a homolog thereof; (ii) the mis-expression of a gene encoding a protein represented by one of SEQ ID Nos: 3 and 4; or (iii) the mis-incorporation of a WT1-binding protein in a transcriptional regulatory complex comprising a WT1 protein. In preferred embodiments: detecting the genetic lesion includes ascertaining the existence of at least one of: a deletion of one or more nucleotides from the WT1-BP gene; an addition of one or more nucleotides to the gene; a substitution of one or more nucleotides of the gene; a gross chromosomal rearrangement of the gene; an alteration in the level of a messenger RNA transcript of the gene; the presence of a non-wild type splicing pattern of a messenger RNA transcript of the gene; or a non-wild type level of the protein.

For example, detecting the genetic lesion can include (i) providing a probe/primer including an oligonucleotide containing a region of nucleotide sequence which hybridizes to a sense or antisense sequence of one of SEQ ID Nos: 1 and 2. or naturally occurring mutants thereof or 5′ or 3′ flanking sequences naturally associated with the WT1-BP gene; (ii) exposing the probe/primer to nucleic acid of the tissue; and (iii) detecting, by hybridization of the probe/primer to the nucleic acid, the presence or absence of the genetic lesion; e.g. wherein detecting the lesion comprises utilizing the probe/primer to determine the nucleotide sequence of the WT1-BP gene and, optionally, of the flanking nucleic acid sequences. For instance, the probe/primer can be employed in a polymerase chain reaction (PCR) or in a ligation chain reaction (LCR). In alternate embodiments, the level of the WT1-binding protein is detected in an immunoassay using an antibody which is specifically immunoreactive with a protein represented by one of SEQ ID Nos: 3 and 4.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### DESCRIPTION OF THE DRAWINGS

FIG. 1 illustrates the pJG4-5 library plasmid and the invariant 107 amino acid moiety it encodes. This moiety carries (amino to carboxy termini) an ATG, an SV40 nuclear localization sequence (PPKKKRKVA), the B42 transcription activation domain, and the HA1 epitope tag (YPYDVPDYA). pJG4-5 directs the synthesis of proteins under the control of the GAL1 promoter. It carries a 2 m replicator and a TRP1⁺ selectable marker. Each of the WT1 binding proteins are inserted as EcoRI-XhoI fragments. Downstream of the XhoI site, pJG4-5 contains the ADH1 transcription terminator.

FIG. 2 illustrates the comparison of Ciao-1 (SEQ ID NO: I) with the WD-40 consensus sequence.

FIG. 3 illustrates the comparison of human par-4 (SEQ ID No: 4) and rat par-4 sequences. The deduced amino acid sequence of human par-4 is compared with that of the rat par-4 protein, with the leucine zipper domain of human and rat par-4 underlined, and the nuclear localization sequence denoted by asterisks. The human par-4 sequence is 75% identical (vertical lines) and 84% similar (dots) to the rat par-4 sequence.

FIG. 4 illustrates the physical interaction between par-4 and WT1.

Panel A shows in vitro transcribed/translated ³⁵S-labeled WT1 and YY1, incubated with 1 μg of either GST-H2-73 (lanes 4&6) or GST alone (lanes 3&5) coupled to glutathione-agarose beads. The input lanes (lanes 1 and 2) were loaded with one-fifth the amount of WT1 and YY1 used in the binding reactions. The presence of a protein with migration identical to that of WT1 input (lane 2) in lane 4 illustrates that WT1 and H2-73 specifically interact in vitro.

Panel B shows that WT1 binds to the leucine repeats of par-4, using in vitro transcribed/translated ³⁵S-labeled WT1 incubated with 1 μg of either GST-par-4 (lane 1), GST-par-4₁₋₂₆₇ (lane 2), GST-par-4₂₆₈₋₃₃₂ (lane 3) or GST alone (lane 4) coupled to glutathione-agarose beads. The input lane (lane 5) was loaded with one-fifth the amount of WT1 used in the binding reactions. A schematic representation of the GST fusion proteins used in the experiment is shown. Black boxes, GST; white boxes, par-4₁₋₂₆₇; shaded boxes, par-4₂₆₈₋₃₃₂. From protein in lane 3, it can be seen that the H2-73 par-4 protein feature defined by amino acid residues 268-332 specifically interacts with WT1 in vitro.

Panel C shows that par-4 binds the zinc fingers of WT1. 293 cells were transfected with 10 μg pCMVFLAGpar-4 and whole cell lysates were prepared. Approximately 200 mg of lysate was incubated with either GST-WT1 (lane 1), GST-WT1₁₋₁₈₀ (lane 2), GST-WT1₁₈₁₋₄₂₉ (lane 3), GST-WT1₁₋₃₀₇ (lane 4), GST-WT1₃₀₈₋₄₂₉ (lane 5) or GST-YY1 (lane 6) coupled to glutathione-agarose beads. Beads were washed, and bound proteins were separated by SDS PAGE, transferred to nitrocellulose/nylon membrane and probed with an anti-FLAG monoclonal antibody. The FLAGpar-4 is indicated by an arrow on the right. The input lane (lane 7) was loaded with approximately 40 μg of lysate. A schematic representation of the GST fusion proteins used in the experiment is shown. Black boxes, GST; white boxes, WT1; shaded box, YY1.

Panel D shows that WT1 and par4 interact in 293 cells. FLAGpar-4 was transfected into 293 cells with (+) or without (−) WT1 as indicated. Immunoprecipitations were performed with a-WT1 or pre-immune rabbit serum (indicated NRS). The immunoprecipitates were analyzed for the presence of FLAGpar-4 by Western blot with anti-FLAG (a-FLAG) monoclonal antibody. Molecular weight markers (kD) are indicated on the left and the position of FLAG-par4 is indicated by an arrow on the right.

Panel E shows the interaction of endogenous WT1 and par-4 in the M15 mouse mesonephric cells. The M15 whole cell extracts were subjected to immunoprecipitation with either pre-immune rabbit serum (NRS) or affinity-purified anti-par-4 (α-par4) antibodies. The immunoprecipitated proteins were analyzed by Western blotting using α-WT1 antibodies. The position of the WT1 protein was indicated by an arrow on the right. The asterisk on the left indicates IgH that cross-reacted with the secondary antibodies. Lane 1: NRS; lane 2: α-par4 antibodies; lane 3: input M15 cell extract.

FIG. 5 illustrates tissue expression of par-4 and WT1 mRNA. Approximately 2 μg of polyA⁺RNA from various human tissues (Clontech) were used for Northern blot analyses. The tissue origins of the RNA samples are indicated across the top of the top panel. The blots were probed with ³²P-labeled par-4 (upper panel), WT1 (middle panel) or βactin (actin, lower panel) cDNA probes. Three major bands (1, 2, 3) of approximately 7.3 kb, 5.0 kb and 2.1 kb respectively detected with the par-4 probe are indicated by arrows on the right. The β-actin probe was used as an internal control for the amount of mRNA loaded onto the gel.

FIG. 6 shows the subcellular expression of par-4 and WT1. Nuclear (N) and cytoplasmic (C) lysates were prepared from cells as follows: Panel A., 293 cells were co-transfected with 10 μg pCMV-FLAG-par-4 and 10 μg PRSV-WT1; Panel B, cells of the mouse mesonephric cell line M 15 were co-transfected with 10 μg pCMV-FLAG-par-4 and 10 μg pRSV-WT1. Proteins were separated by SDS-PAGE and detected by Western blot using antibodies (a) α-FLAG, α-WT1 and α-p70^{s6k} antibodies for A., and affinity purified α-par-4 polyclonal antibody, α-WT1 and α-p70^{s6k} antibodies respectively for B. Molecular weight markers (kD) are indicated on the left.

FIG. 7 shows that par-4 specifically modulates the transcriptional activity of WT1. Results from all CAT assays represent the means and standard deviations from three independent transfections and CAT assays.

Panel A shows that par-4 inhibits WT1-mediated transcriptional activation. A reporter plasmid containing three WT1/EGR1 binding sites was co-transfected with pRSV-WT1 (A) and increasing amounts of pCMV-par-4 expression plasmid (lanes 6-9), or with the expression plasmid alone (lanes 10 and 11). The amounts of transfected plasmids are indicated.

Panel B shows that par-4 does not affect EGR1-mediated transcriptional activation. The same reporter plasmid described in (A) was co-transfected with pCMV-EGR1 and increasing amounts of pCMV-par-4 expression plasmid (lanes 4 and 5), or with the expression plasmid alone (lanes 6 and 7).

Panel C shows that cotransfection of par-4 does not change WT1 expression levels. Cells were transfected with 10 μg pRSV-WT1 and 1-15 μg pCMV-par-4 (lanes 1-5). Whole cell lysates were prepared and WT1 and par-4 were detected by Western blotting. Molecular weight markers (kDa) are shown on the left.

Panel D shows that a mutant par-4 protein lacking the WT1-interacting domain does not affect WT1-mediated transcriptional activation. The same reporter plasmid described in (A) was co-transfected with WT1 and expression plasmids encoding full length par-4 (lane 7) or par-4 lacking the WT-1-interacting domain (lane 8), or with vector plasmid alone (lane 9). The amount of transfected plasmids is indicated.

FIG. 8 illustrates par-4 augmentation of WT-1-mediated transcriptional repression. A reporter plasmid containing five GAL4 DNA binding sites (pGAL4TKCAT) was co-transfected with different combinations of plasmids indicated in the figure under “transfected DNA” as is the amount of transfected plasmids. Results for all CAT assays represent the means and standard deviations from three independent transfections and CAT assays.

FIG. 9 illustrates that par-4 is a transcriptional repressor. CAT reporter plasmids with (GAL4TKCAT) and without (TKCAT) GAL4 DNA binding sites were co-transfected with GAL4-par-4 (lanes 5-9 and 11) or GAL4 (lanes 2-4 and 12). The amount of transfected plasmids is indicated. Results represent the means and standard deviations from three independent transfections and CAT assays.

FIG. 10 illustrates that par-4 overcomes WT1-induced growth suppression of A375-C6 cells. A375-C6 cells were transfected separately with CMV vector, CMV-WT1 (WT1), CMV-par-4 (Par-4), CMV-par-4₁₋₂₆₇; or cotransfected with CMV-WT1 plus CMV-par-4 (WT1+Par4) or CMV-WT1 plus CMV-par-41267. Stably transfected clones were selected with G418 sulfate and pools of transfected clones were maintained as cell lines L1 or L2. The transfected cells were seeded in 96-well plates and cultured for 72 hr. Thereafter, the cells were pulsed with [³H]thymidine for 8 h and incorporation of [³H]thymidine was determined. A total of three separate experiments were performed. Data shown indicate mean of counts per minute from 12 different observations; error bars indicate standard deviations.

### DETAILED DESCRIPTION OF THE INVENTION

Wilm's Tumor has been linked to the inactivation of the WT1 tumor-suppressor gene at the 11p13 chromosomal locus (Haber et al. (1992) Adv Cancer Res 59:41-68). WT1 encodes a developmentally regulated transcription factor of 52-54 kDa. The C terminus of WT1 contains four zinc finger domains of the Cys₂-His₂ type (Call et al. (1990) Cell 60:509-520), which apparently confers sequence-specific binding to DNA. The WT1 amino terminus is rich in proline residues, a feature found in the transactivation domain of several transcription factors. The function of WT1 is evidently required for initiation and maintenance of diverse programs of differentiation in both embryonic and adult tissues, particularly in the urogenital system. For instance, WT1 is involved in the mesenchymal-epithelial differentiation program in the kidney and in the stromal fibroblast-epithelial program in the uterus.

The apparent mechanism of action of the WT1 gene products involves regulating transcription of genes related to growth and/or differentiation. Repression of transcription initiated from the promoters of, for example, the PDGF A-chain, EGR-1, and IGF-II genes is a general paradigm for the tumor suppressor activity of WT-1 and indicates ability of the protein to directly mediate organogenesis through its influence on the transcription of growth-related genes. However, recent evidence suggests that, in some cellular contexts, WT1 can function as an activator of transcription and that repression mediated by WT1 may be due in part to its physical association with the p53 protein (Maheswaran et al. (1993) PNAS 90:5100-5104). It has been demonstrated, for example, that in p53 deficient cells transcriptional activation by WT1 occurs from genes containing WT1 binding sites in their promoter regions, whereas repression results upon expression of p53. Moreover, it has also been shown that WT1 is composed of separate regulatory domains that function either to activate or suppress transcription (Wang et al. (1993) J Biol Chem 268:9172-9175). We hypothesized that the transcriptional regulatory control by WT1, both as a transcriptional activator and repressor, is likely to be modulated by other cellular factors of which p53 is exemplary.

As described herein, a WT1-dependent interaction trap assay was used to identify cellular proteins that can associate with the human WT1 protein. Surprisingly, it was observed that WT1 was able to associate with itself to form at least binary complexes. In addition, a number of novel proteins which interact with WT1 were cloned from a human cDNA library. Given the apparent role of WT1 in mediating both transcriptional activation and repression, the present invention is consistent with WT1 being an important core protein of various multimeric complexes, with multiple cellular proteins participating in WT1 assembled complexes to control the activation and inactivation of growth and developmental gene programs. Thus, WT1 can, depending on the proteins associated in a complex with it, repress growth-related genes while activating genes that are involved in, for example, epithelial differentiation. This invention derives in part from the discovery that, in addition to the tumor suppressor protein p53, the Wilms tumor suppressor protein WT1 is also associated with several other cellular proteins (hereinafter termed “cellular WT1-binding proteins” or “WT1-binding proteins” or “WT1-BPs”), which association is likely to be involved in WT1-mediated gene expression and presumably important in the pathogenesis of Wilms tumor disease states as well as other proliferative and differentiative disorders. For example, association of one or more of the subject WT1-binding proteins with WT1 can be important for initiating and establishing diverse programs of differentiation, as well as for providing a mechanism to ensure developmentally coordinated action of WT1. Consequently, the interaction of WT1 with one or more of the subject WT1-binding proteins are significant in the modulation of cellular homeostasis, in the control of organogenesis, in the control of entry into the apoptosis pathway, in the maintenance and survival of differentiated tissues, as well as in development of Wilms tumor and other neoplastic abnormalities.

Accordingly, certain aspects of the present invention relate to diagnostic and therapeutic assays and reagents for detecting and treating disorders involving aberrant assembly of WT1 complexes. A preferred diagnostic test is for duplication of the par-4 gene chromosome IV, or for other chromosomal duplications, insertions, deletions, inversions, transpositions, or point mutations of genes encoding WT1 binding proteins. Moreover, drug discovery assays are provided for identifying agents which can modulate the binding of one or more of the subject WT1-binding proteins with WT1 or other transcriptional regulatory proteins. Such agents can be useful therapeutically to alter the growth and/or differentiation of a cell, but can also be used in vitro as cell-culture additives for controlling proliferation and/or differentiation of cultured cells and tissues. Other aspects of the invention are described below or will be apparent to those skilled in the art in light of the present disclosure.

One of the novel proteins, Ciao-1 (“Bridge” in Chinese), which was shown to physically interact with WT1, was found to contain areas of distinct similarity with a family of proteins that contained the so-called WD-40 for β-transduction repeats (Fong et al. (1986) PNAS 83:2162-2166). The WD-40 repeating unit was originally defined (by Fong et al., supra) in the P-subunit of the signal transducing GTP-binding proteins, and was later found in many proteins of diverse biological functions including signal transduction. cell-cycle regulation, splicing, and transcription (reviewed in van der Voorn et al. (1992) FEBS Lett 307:131-134). It is noteworthy that among the identified WD-40 family members are two proteins involved in transcription, the Drosophila TAPF80 (Dynlacht et al. (1993) Nature 363:176-179) and yeast Tup1 (Williams et al. (1990) Mol Cell Biol 10:6500-6511). TAF80 is a component of the large basal transcription factor complex TFIID (Dynlacht et al, supra). Thus, its role in transcription is evident. Tup1, on the other hand, functions as a transcriptional repressor when complexed with another protein, Ssn6 (Williams et al. (1991) Mol Cell Biol 11:3307-3316). Genetic studies suggest Tup1-Ssn6 to be a general repressor that is involved in the repression of diverse sets of genes in yeast, including mating type A-specific, haploid-specific and glucose-repressible genes (Keleher et al. (1992) Cell 68:709-719). Tup1-Ssn is presumably recruited to glucose-repressible promoters via its interaction with a sequence-specific DNA-binding protein Mig1 (Keleher et al., supra), which was reported to be related to WT1 (Nehlin et al. (1990) EMBO J 9:2891-2898). Accordingly, in light of the facts that Ciao-1 is a WD-40 protein with similarities to Tup1, and that it interacts with WT1, which has previously been shown to be related to Mig1, it is possible that a transcriptional repression pathway involving Tup1-like proteins may be operative in mammalian cells. In this scenario, Ciao-1 could be considered as a functional homolog of Tup1, despite the observation that the structures of the two proteins are not entirely similar.

For convenience, certain terms employed in the specfication, examples, and appended claims are collected here.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, such as peptide nucleic acids and phosphoro-thioate nucleic acids, and, as applicable to the embodiment being described, single-stranded (such as sense or antisense) and double-stranded polynucleotides.

As used herein, the term “gene” or “recombinant gene” refers to a nucleic acid comprising an open reading frame, encoding, for example, a WT1-binding protein of the present invention, including both exon and (optionally) intron sequences. A “recombinant gene” refers to nucleic acid encoding, for example, a WT1-binding protein and comprising WT1-BP encoding exon sequences. It can optionally include intron sequences derived from a chromosomal WT1-BP gene or from an unrelated chromosomal gene. A recombinant gene can be substantially isolated, or it can be chemically or enzymatically joined to one or more other nucleic acid sequences, different from the sequence context in which it is found in nature. Exemplary recombinant genes encoding the subject WT1-binding proteins are represented by any one of SEQ ID Nos: 1 and 2. The term “intron” refers to a DNA sequence present in a given WT1-BP gene which is generally found between exons and not translated into protein.

As used herein, the term “transfection” means the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of the WT1-binding protein of the present invention or where anti-sense expression occurs from the transferred gene, the expression of a naturally-occurring form of the WT1-binding protein is disrupted.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

“Transcriptional regulatory sequence” is a generic term used throughout the specification to refer to a DNA sequence, such as an initiation signal, an enhancer, and a promoter, which induce or control transcription of protein coding sequences with which they are operably linked. In preferred embodiments, transcription of a recombinant WT1-BP gene is under the control of a promoter sequence (or other transcriptional regulatory sequence) which controls the initiation and extent of expression of the recombinant gene in a cell-type in which expression is intended. It will also be understood that the recombinant gene can be under the control of transcriptional regulatory sequences which are the same or which are different from those sequences which control transcription of the naturally-occurring form of the WT1-binding protein.

As used herein, the term “tissue-specific promoter” means a DNA sequence that serves as a promoter, i.e., regulates expression of a selected DNA sequence operably linked to the promoter, and which effects preferential expression of the selected DNA sequence in specific cells of a tissue, such as cells of a urogenital origin, e.g. renal cells, or cells of a neural origin, e.g. neuronal cells. The term also covers so-called “leaky” promoters, which regulate expression of a selected DNA primarily in one tissue, and cause expression at a lower level in other tissues as well.

As used herein, a “transgenic animal” is any animal, preferably a non-human mammal, a bird or an amphibian, in which one or more of the cells of the animal contain heterologous or foreign nucleic acid introduced by way of human intervention, such as by trangenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule can be integrated within a chromosome, or it can be extrachromosomally replicating DNA. In the typical transgenic animals described herein, the transgene causes cells to express a recombinant form of a subject WT-1-binding protein, e.g. either agonistic or antagonistic forms. However, transgenic animals in which the recombinant WT1-BP gene is silent are also contemplated, as for example, the FLP or CRE recombinase dependent constructs described below. The “non-human animals” of the invention include vertebrates such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding, for example, embryogenesis and tissue patterning. The term “chimeric animal” is used herein to refer to animals in which a recombinant gene is found, or in which the recombinant gene is expressed in some but not all cells of an animal. The term “tissue-specific chimeric animal” indicates that a recombinant gene, for example, the recombinant WT1-BP gene, is present and/or expressed in some tissues but not others.

As used herein, the term “transgene” means a nucleic acid sequence (encoding, e.g., a WT1-binding protein), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location different from that of the natural gene or its insertion results in a knockout). A transgene can include one or more transcriptional regulatory sequences and any other nucleic acid, such as introns, that may be necessary for optimal expression of a selected nucleic acid.

As is well known, a gene for a particular polypeptide can exist in single or multiple copies within the genome of an individual. Such duplicate genes can be identical or can have certain modifications, including nucleotide substitutions, additions or deletions, which all still code for polypeptides having substantially the same activity. The term “DNA sequence encoding a WT1-binding protein” can thus refer to one or more genes within a particular individual. In addition to the human ciao-1 and par-4 nucleotide sequences shown in SEQ ID No: 1 and SEQ ID No: 2, respectively, it will be appreciated by those skilled in the art that DNA sequence polymorphisms that lead to changes in the amino acid sequences of these proteins can exist within a population. Such genetic polymorphisms in the ciao-1 and par-4 genes can exist among individuals within a population due to mutation and maintenance of neutral mutations, which leads to natural allelic variation, which typically can comprise 1-5% variance in the nucleotide sequence of the a gene. Any and all such nucleotide variations and resulting amino acid polymorphisms in ciao-1 and par-4 that are the result of natural allelic variation are intended to be within the scope of the invention.

Moreover, nucleic acid molecules encoding ciao-1 and par-4 proteins from other species, and thus which have a nucleotide sequence that differs from the human sequence of SEQ ID No: 1 or from SEQ ID No: 2 but that is related to the human sequence, are intended to be within the scope of the invention. Nucleic acid molecules corresponding to natural allelic variants and nonhuman homologues of the human cDNA of the invention can be isolated based on their homology to the human nucleic acid molecules disclosed herein using the human cDNA, or a portion thereof, as a hybridization probe according to standard hybridization techniques under stringent hybridization conditions. Accordingly, in another embodiment, an isolated nucleic acid molecule of the invention hybridizes under stringent conditions to the nucleic acid molecule comprising the nucleotide sequence of SEQ ID NO: 1. Further, in another embodiment, an isolated nucleic acid molecule of the invention hybridizes under stringent conditions to the nucleic acid molecule comprising the nucleotide sequence of SEQ ID No: 2. In certain embodiment, the nucleic acid is at least 150, 300, 600, 1200, 1500, 2000, or 3000 nucleotides in length. Preferably, an isolated nucleic acid molecule of the invention that hybridizes under stringent conditions to the sequence of SEQ ID No: 1 corresponds to a naturally-occurring nucleic acid molecule. Further, an isolated nucleic acid molecule of the invention that hybridizes under stringent conditions to the sequence of SEQ ID No: 2 corresponds to a naturally-occurring nucleic acid molecule. In on embodiment, the nucleic acid encodes natural human ciao-1 protein. In another embodiment, the nucleic acid molecule encodes a natural human par-4 protein.

“Homology” refers to sequence similarity and extent of identity between two peptides or between two nucleic acid molecules. Homology can be determined by comparing a position in each sequence which may be aligned for purposes of comparison. When a position in the compared sequence is occupied by the same base or amino acid, then the molecules are homologous (identical) at that position. A degree of homology between sequences is a function of the number of matching or homologous positions shared by the sequences.

To determine the percent homology of two amino acid sequences (e.g., SEQ ID No: 1 and a mutant or homologous form thereof), the sequences are aligned for optimal comparison purposes (e.g., gaps may be introduced in the sequence of one protein for optimal alignment with the other protein). The amino acid residues at corresponding amino acid positions are then compared. When a position in one sequence (e.g., SEQ ID No: 1) is occupied by the same or a similar amino acid residue as the corresponding position in the other sequence (e.g., a mutant form of ciao-1), then the molecules are homologous at that position (i.e., as used herein amino acid “homology” is equivalent to amino acid identity or similarity). As used herein, an amino acid residue is “similar” to another amino acid residue if the two amino acid residues are members of the same family of residues having similar side chains.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric protein” or “fusion protein” is a fusion of a first amino acid sequence encoding one of the subject WT1-binding proteins with a second amino acid sequence defining a domain foreign to and not substantially homologous with any domain of the subject WT1-BP. A chimeric protein can present a foreign domain which is found (albeit in a different protein) in an organism which also expresses the first protein, or it may be an “interspecies”, “intergenic”, etc. fusion of protein structures that are naturally expressed by different species of organisms.

The term “evolutionarily related to”, with respect to nucleic acid sequences encoding WT-1-binding protein, refers to nucleic acid sequences which have arisen naturally in an organism, including naturally occurring mutants. The term also refers to nucleic acid sequences which, while derived from a naturally occurring WT-1-BP, have been altered by mutagenesis, as for example, combinatorial mutagenesis described below, yet still encode polypeptides which have at least one activity of a WT1-binding protein.

The term “isolated” as also used herein with respect to proteins or nucleic acids, such as DNA or RNA, refers to molecules separated from other proteins, DNAs, or RNAs, respectively, that are present in the natural source of the macromolecule. For example, an isolated nucleic acid encoding one of the subject WT1-binding proteins preferably includes no more than 10 kilobases (kb) of nucleic acid sequence which naturally immediately flanks that particular WT1-BP gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

As used herein, the terms peptide, polypeptide, and protein are, unless specified otherwise, used interchangeably. Peptides, polypeptides, and proteins used in methods and compositions described herein can be recombinant, synthesized on ribosomes in a cell-free in vitro system, purified from natural sources, or chemically synthesized. For example, reference to the use of a bacterial protein or a protein from bacteria, includes the use of recombinantly produced molecules, molecules purified from natural sources, or chemically synthesized molecules.

As described below, one aspect of the invention pertains to an isolated nucleic acid having a nucleotide sequence encoding one of the subject WT1-binding proteins, and/or equivalents of such nucleic acids. The term nucleic acid as used herein is intended to include fragments and equivalents. The term equivalent is understood to include nucleotide sequences encoding functionally equivalent WT1-binding proteins or functionally equivalent peptides which, for example, retain the ability to bind to WT1. Equivalent nucleotide sequences will include sequences that differ by one or more nucleotide substitutions, additions or deletions, such as allelic variants; and will, therefore, include sequences that differ from the nucleotide sequence of WT1-BP genes shown in any of SEQ ID Nos: 1 and 2 due to the degeneracy of the genetic code. Equivalents will also include nucleotide sequences that hybridize under stringent conditions (i.e., equivalent to about 20-27° C. below the melting temperature (Tₘ) of the DNA duplex formed in about 1 M salt) to the nucleotide sequence of the subject WT1-bp genes represented in SEQ ID Nos: 1 and 2, or to the nucleotide sequence of a WT1-bp gene from the pSHI-WT1BP library. In one embodiment, equivalents will further include nucleic acid sequences derived from and evolutionarily related to, a nucleotide sequences shown in any of SEQ ID Nos: 1 and 2.

Moreover, it will be generally appreciated that, under certain circumstances, it can be advantageous to provide homologs of the subject WT1-binding proteins which function in a limited capacity as one of either a WT1-BP agonists or a WT1-BP antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of a WT1-binding protein's biological activities. For instance, antagonistic homologs can be generated which interfere with the ability of certain of the wild-type (“authentic”) WT-binding proteins to form complexes with WT1, but which do not substantially interfere with the formation of complexes between the WT1-BP and other cellular proteins, such as can be involved in other transcriptional regulatory mechanisms of the cell.

Homologs of the subject WT1-binding proteins can be generated by mutagenesis, such as by discrete point mutation(s), or by truncation. For instance, mutation can give rise to homologs which retain substantially the same, or merely a subset, of the biological activities of the WT1-BP from which it was derived. Alternatively, antagonistic forms of the protein can be generated which are able to inhibit the function of the naturally occurring form of the protein, such as by competitively binding to WT1.

A polypeptide is considered to possess a biological activity of a WT1-binding protein if the polypeptide has one or more of the following properties: the ability to modulate gene expression, e.g. of a developmentally or growth regulated gene; the ability to modulate WT1-mediated gene expression in a tissue in which the WT1 protein is expressed, e.g. in urogenital tissue such as bladder, in gonadal tissues (such as ovarian or testicular), in renal tissue, in mesothelium, in hematopoietic cells, or in neural tissue; the ability to bind to a WT1 protein; the ability to agonize or antagonize assembly of WT1-containing transcriptional protein complexes such as may be involved in WT-1-dependent inactivation or, alternatively, activation of gene transcription, e.g. to modulate expression of genes involved in mesenchyme-to-epithelial transition, e.g. to modulate differentiation of mesodermally-derived tissue, such as tissue derived from lateral mesoderm or intermediate mesoderm, or alternatively, ectodermally-derived spinal or brain tissue; the ability to modulate differentiation of renal tissue in normal kidneys or in Wilm's tumors; the ability to modulate differentiation of urogenital tissue, such as normal or neoplastic gonadal tissue, e.g. ovarian tissue, e.g. gonadal tissue (such as granulosa and Sertoli cells); the ability to modulate differentiation of heart mesothelium; and/or the ability to modulate neural differentiation. A protein also has biological activity if it is a specific agonist or antagonist of one of the above recited properties.

Preferred nucleic acids encode a WT1-binding protein comprising an amino acid sequence at least 60% homologous, more preferably at least 70% homologous and most preferably at least 80% homologous with an amino acid sequence shown in one of SEQ ID Nos: 3 and 4. Nucleic acids which encode polypeptides having an activity of a subject WT-1-binding protein and having at least about 90%, more preferably at least about 95%. and most preferably at least about 98-99% homology with a sequence shown in one of SEQ ID Nos: 3 and 4 are also within the scope of the invention, as of course are proteins which are identical to the aforementioned sequence listings. In one embodiment, the nucleic acid is a cDNA encoding a peptide having at least one activity of a subject WT1-binding protein. Preferably, the nucleic acid is a cDNA molecule comprising at least a portion of the nucleotide sequence represented in one of SEQ ID Nos: 1 and 2. A preferred portion of these cDNA molecules includes the coding region of the gene. For instance, a recombinant WT-1-BP gene can include nucleotide sequences of a PCR fragment generated by amplifying the coding sequences for one of the WT1-BP clones using sets of primers derived from Table 3 below.

Certain of the nucleotide sequences shown in the appended sequence listing encode portions of the subject WT-1-binding proteins. Therefore, in a further embodiment of the invention, the recombinant WT1-BP genes can include, in addition to nucleotides shown in SEQ. ID. Nos: 1 and 2 encoding the amino acid sequences shown in SEQ. ID. Nos: 3 and 4, other nucleotide sequences which encode amino acids at the C-terminus and/or N-terminus of each protein.

Another aspect of the invention provides a nucleic acid which hybridizes under high or low stringency conditions to a DNA or RNA which encodes a peptide having all or a portion of an amino acid sequence shown in one of SEQ ID No: 3 or SEQ ID No:4. Appropriate stringency conditions which promote DNA hybridization, for example, 6.0×sodium chloride/sodium citrate (SSC) at about 45° C., followed by a wash of 2.0×SSC at 50° C., are known to those skilled in the art or can be found in Ausubel, F, et al. Eds. Current Protocols in Molecular Biology, John Wiley & Sons, N.Y. (1989), 6.3.1-6.3.6. For example, the salt concentration in the wash step following hybridization can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C.

Nucleic acids having a sequence which differs from the nucleotide sequence shown in any of SEQ ID Nos: 1 and 2 due to degeneracy in the genetic code are also within the scope of the invention. Such nucleic acids encode functionally equivalent peptides (i.e., a peptide having a biological activity of a WT1-binding protein) but that differ in sequence from said sequence listings due to degeneracy in the genetic code. For example, a number of amino acids are designated by more than one triplet. Codons that specify the same amino acid, or synonyms (for example, CAU and CAC each encode histidine) may result in “silent” mutations of the nucleotide sequence which do not affect the amino acid sequence of the WT1-binding protein. However, it is expected that DNA sequence polymorphisms that do lead to changes in the amino acid sequences of the subject WT1-binding proteins will exist among vertebrates. One skilled in the art will appreciate that these variations in one or more nucleotides (up to about 3-5% of the nucleotides) of the nucleic acids encoding polypeptides having an activity of a WT1-binding protein may exist between individuals of a given species due to natural allelic variation. Any and all such nucleotide variations and resulting amino acid polymorphisms are within the scope of this invention.

Fragments of the nucleic acids encoding an active portion of the subject WT1-binding proteins are also within the scope of the invention. As used herein, a fragment of the nucleic acid encoding the active portion of a WT1-binding protein refers to an oligonucleotide having fewer nucleotides than the nucleotide sequence encoding the entire amino acid sequence of a WT1-binding protein but which nevertheless encodes a peptide having a WT1-BP biological activity, e.g. the fragment retains the ability to bind to a WT1 protein. Nucleic acid fragments within the scope of the present invention include those capable of hybridizing under high or low stringency conditions with nucleic acids from other species such as can be used in screening protocols to detect WT1-BP homologs, as well as probes capable of hybridizing with nucleic acids from human specimens for use in detecting the presence of a nucleic acid encoding one of the subject WT1-BPs, including alternate isoforms, e.g. mRNA splicing variants. Nucleic acids within the scope of the invention can also contain linker sequences, modified restriction endonuclease sites and other sequences that are added as they are useful for molecular cloning, expression or purification of recombinant forms of the subject WT1-binding genes and encoded proteins.

As indicated by the examples set out below, a nucleic acid encoding a WT1-binding protein or a homologous gene thereof can be obtained from mRNA present in any of a number of different types of eukaryotic cells. It should also be possible to obtain nucleic acids encoding WT1-binding proteins of the present invention from genomic DNA obtained from both adults and embryos. For example, a gene encoding a WT-1-binding protein can be cloned from either a cDNA or a genomic library in accordance with protocols herein described, as well as those generally known to persons skilled in the art. A cDNA encoding a WT-1-binding protein can be obtained by isolating total mRNA from a cell, e.g. a mammalian cell, e.g. a human cell, including tumor cells. Double stranded cDNAs can then be prepared from the total mRNA, and subsequently inserted into a suitable plasmid or bacteriophage vector using any one of a number of known techniques. The gene encoding the WT-1-binding protein can also be cloned using established polymerase chain reaction techniques in accordance with the nucleotide sequence information provided by the invention. The nucleic acid of the invention can be DNA or RNA. A preferred nucleic acid is a Ciao-1 cDNA represented by the sequence shown in SEQ ID No: 1. Another nucleic acid is a par-4 cDNA represented by the sequence shown in SEQ ID No: 2. Other preferred nucleic acids include cDNA molecules represented by the sequences shown in one of SEQ ID Nos: 1 and 2. A preferred nucleic acid is a cDNA derived from the pSHI-WT1BP library.

Another aspect of the invention relates to the use of the isolated nucleic acid in “antisense” therapy. As used herein, “antisense” therapy refers to administration of an exogenous oligonucleotide, or in situ generation of an oligonucleotide or its derivative, which specifically hybridizes (e.g. binds) under cellular conditions, with the cellular mRNA and/or genomic DNA encoding a WT1-binding protein so as to inhibit expression of that protein, as for example by inhibiting transcription and/or translation. The binding can be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a WT1-binding protein. Alternatively, the antisense construct can be an oligonucleotide probe which is generated ex vivo and which, when introduced into the cell causes inhibition of expression by hybridizing with the mRNA and/or genomic sequences of a WT1-BP gene. Such oligonucleotide probes are preferably modified oligonucleotides which are resistant to endogenous nucleases, e.g. exonucleases and/or endonucleases, and is therefore stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by van der Krol et al. (1988) Biotechniques 6:958-976; and Stein et al. (1988) Cancer Res 48:2659-2668.

Accordingly, the modified oligomers of the invention are useful in therapeutic, diagnostic, and research applications. In therapeutic applications, the oligomers are utilized in a manner appropriate for gene therapy and for antisense therapy in general. For such therapy, the oligomers of the invention can be formulated for a variety of loads of administration, including systemic and topical or localized administration. Techniques and formulations generally can be found in Remmington's Pharmaceutical Sciences, Meade Publishing Co., Easton, Pa. For systemic administration, injection is preferred, including intramuscular, intravenous, intraperitoneal, and subcutaneous for injection, the oligomers of the invention can be formulated in liquid solutions, preferably in physiologically compatible buffers such as Hank's solution or Ringer's solution. In addition, the oligomers can be formulated in solid form and dissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents can be used to facilitate permeation. Transmucosal administration can be accomplished through nasal spray or by use of suppositories. For oral administration, the oligomers are formulated into conventional oral administration forms such as capsules, tablets, and tonics. For topical administration, the oligomers of the invention are formulated into ointments, salves, gels, or creams as generally known in the art.

In addition to use in therapy, the oligomers of the invention can be used as diagnostic reagents to detect the presence or absence of the target DNA or RNA sequences to which they specifically bind. Such diagnostic tests are described in further detail below.

Likewise, the antisense constructs of the present invention, by antagonizing the normal biological activity of a WT1-binding protein, can be used in the manipulation of tissue, e.g. tissue differentiation, both in vivo and in ex vivo tissue cultures.

This invention also provides expression vectors containing a nucleic acid encoding a peptide having an activity of a WT1-binding protein, operably linked to at least one transcriptional regulatory sequence. Operably linked is intended to mean that the nucleotide sequence is linked to a regulatory sequence in a manner which allows expression of the nucleotide sequence. Regulatory sequences are art-recognized and are selected to direct expression of a recombinant WT1-binding protein. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences, that is, sequences that control the expression of a DNA sequence when operatively linked to that DNA sequence, can be used in these vectors to express DNA sequences encoding the WT1-binding proteins of the present invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. It is advantageous to design of expression vector in light of attributes such as choice of a host cell to be transformed, and the type of protein to be expressed. Moreover, the vector's copy number, the ability to control that copy number and the expression of any other proteins encoded by the vector, such as antibiotic markers, should also be considered. In one embodiment, the expression vector includes a recombinant gene encoding a polypeptide which mimics or otherwise agonizes the action of a WT1-binding protein, or alternatively, which encodes a polypeptide that antagonizes the action of an authentic WT1-binding protein. Such expression vectors can be used to transfect cells and thereby produce polypeptides, including fusion proteins, encoded by nucleic acids as described herein.

Moreover, the gene constructs of the present invention can also be used as a part of a gene therapy protocol to deliver nucleic acids encoding either an agonistic or antagonistic form of one or more of the subject WT1-binding proteins. Thus, another aspect of the invention features expression vectors for in vivo transfection and expression of a WT1-binding protein in particular cell types so as to reconstitute the function of or alternatively, abrogate the function of one or more of the subject WT1-binding proteins in a cell in which that protein or other transcriptional regulatory proteins to which it bind are misexpressed. For example, gene therapy can be used to deliver a gene encoding a WT-binding protein which inhibits neoplastic transformation, such as in the generation of Wilm's tumor, by interfering with the biological function of WT1.

Expression constructs of the subject WT1-binding proteins, and mutants thereof, can be administered in any biologically effective carrier, e.g. any formulation or composition capable of effectively delivering the WT1-BP gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramicidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically. Furthermore, it will be recognized that the particular gene construct provided for in vivo transduction of WT1-BP expression are also useful for in vitro transduction of cells, such as for use in a diagnostic assays.

A preferred approach for in vivo introduction of nucleic acid into a cell is by use of a viral vector containing nucleic acid, e.g. a cDNA, encoding the WT1-binding protein or homolog thereof Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up the vector.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding one of the subject receptors rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:41044115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

In choosing a retroviral vector as a gene delivery system for the subject WT1-BP gene to a target cell, a prerequisite for the successful infection of the target cell by most retroviruses, and thus of stable maintenance of the recombinant WT1-BP gene, is that the target cell must be a dividing cell. Such a requirement to use of retroviral vectors is met with most target cells for the present invention. The gene therapy constructs of the present invention are intended for cells, for example, of a Wilm's tumor, for example for use of antagonistic forms of WT1-BP^{Ciao-1} or WT-1-BP^{par-4}. The limitation on infection of dividing cells is beneficial, in that surrounding tissue comprising nontransformed cells do not undergo as rapid cell division as target tumor cells, and is therefore more refractory to infection with a retroviral vector.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) PNAS 86:9079-9083; Julan et al. (1992) J. Gen Virol 73:3251-3255; and Goud et al. (1983) Virology 163:251-254); or coupling cell surface receptor ligands to the viral env proteins (Nedaet al. (1991) J Biol Chem 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector in to an amphotropic vector.

Moreover, use of retroviral gene delivery can be further enhanced by the use of tissue- or cell-specific transcriptional regulatory sequences which control expression of the WT1-BP gene of the retroviral vector.

Another viral gene delivery system useful in the present invention utilitizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the inserted WT1-BP gene can be under control of, for example, the E1A promoter, the major late promoter (MLP) and associated leader sequences, the E3 promoter, or exogenously added promoter sequences.

Yet another viral vector system useful for delivery of the subject WT1-BP genes is the adeno-associated virus (AAV). Adeno-associated virus is a naturally occurring defective virus that requires a helper virus, such as an adenovirus or a herpes virus, for replication and productive growth. (For a review see Muzyczka et al. Curr. Topics in Micro. and Immunol. (1992) 158:97-129). It is also one of the few viruses capable of integrating its DNA into non-dividing cells with high frequency of stable integration (see for example Flotte et al. (1992) Am. J Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Up to about 4.5 kb of exogenous DNA can be incorporated. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J Biol. Chem. 268:3781-3790).

In addition to viral transfer methods, such as those illustrated above, non-viral methods can also be employed to cause expression of a WT1-binding protein in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject WT1-BP gene by the targeted cell. Exemplary gene delivery systems of this type include liposomal derived systems, poly-lysine conjugates, and artificial viral envelopes.

In a representative embodiment, a therapeutic WT1-BP gene can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075). For example, lipofection of Wilms tumor cells can be carried out using liposomes tagged with monoclonal antibodies against, for example, the Thy-1 antigen, the cell adhesion molecule NCAM, carbohydrate antigen 125 (CA125), or any other cell surface antigen present on the tumor cells.

In clinical settings, the gene delivery systems for the therapeutic WT1-BP gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal, being quite localized to a particular tissue or organ. For example, the gene delivery vehicle can be introduced by catheter (see U.S. Pat. No. 5,328,470) or by stereotactic injection (e.g. Chen et al. (1994) PNAS 91: 3054-3057). To illustrate, an antagonistic form of one of the subject WT1-binding proteins, such as the fragment of the Ciao-1 clone described in the examples below, can be delivered in a gene therapy construct to a cell by electroporation using techniques described, for example, by Dev et al. ((1994) Cancer Treat Rev 20:105-115).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

A wide variety of pathological cell proliferative conditions present targets for the gene constructs of the present invention to provide therapeutic benefits. The general strategy is the inhibition of aberrent gene expression or repression mediated by WT1 or other transcriptional regulatory proteins with which the subject WT1-binding proteins interact. More generally, however, another aspect of the present invention relates to a method of inducing and/or maintaining a differentiated state, enhancing survival and/or affecting proliferation of a cell in which one of the subject WT-1-binding proteins is expressed, by contacting the cell with an agent which modulates the function (as an agonist or an antagonist) of the WT1-binding protein. For instance, it is contemplated by the invention that, in light of the apparent broad involvement of WT1 in the formation of ordered spatial arrangements of various tissues in vertebrates, as well as other possible biological functions which can be ascribed to the subject WT1-binding proteins based on homology, the subject method could be used to generate and/or maintain an array of various tissue both in vitro and in vivo. For instance, modulation of the function of one or more of the subject WT1-binding proteins can be employed in both cell culture and therapeutic methods involving generation and maintenance of renal tissue, and also for non-renal tissue, such as in controlling the development and maintenance of tissue from the digestive tract, pancreas, spleen, lungs, and other organs which derive from the primitive gut, as well as lateral and intermediate mesoderm-derived structures including urogenital structures and mesothelia; and certain ectodermally-derived tissues, such as spinal and brain tissue. The agent can be, as appropriate, any of the preparations described herein, including gene therapy constructs, antisense molecules, peptidomimetics or other agents identified in the drug screening assays provided herein. For purposes of the description of therapeutic inventions and the like, as well as drug screening assays, the term WT1-binding protein can be further understood to include WT1 itself, which, as described below, is able to bind to itself.

In an exemplary embodiment, the present method can be used in the treatment of hyperplastic and neoplastic disorders, particularly those characterized by mis-expression of proteins involved in regulatory complexes which include the subject WT-1-binding proteins, e.g., complexes involving WT1. For instance, nephroblastomas (e.g. Wilms tumors) are marked by abnormal proliferation of renal cells in which alteration in WT1 function is implicated in the pathogenesis of tumor progression. The change in WT1 regulatory function is presumbaly due to mechanisms which involve either disruption of WT1 repressor activity, e.g., of growth-related genes, or inhibition of WT1-mediated transcriptional acitvation, e.g. of differentiative gene programs, or both. For cells in which WT1 is mis-expressed, e.g. deleted or mutated, compensatory homologs of the subject WT1-binding proteins may be useful to offset the WT1 mutation. Alternatively, modulation of interactions between one or more of the subject WT1-binding proteins and WT1, or other transcriptional regulatory proteins, may result in apoptosis or mitotic catastrophe of the transformed cells. Modulation with low molecular weight drugs that interact with proteins and selected by the methods described here are contemplated for a variety of therapeutic applications including suppression of tumors and metastases and neurological defects.

Furthermore, WT1 is deleted or mutated in only 10 percent of sporadic Wilms tumors, implicating other proteins which act close to WT1 as potential sites for transforming genetic lesions. Accordingly, each of the subject WT1-binding proteins are potential alternate loci in the development of Wilms tumor. In those situations, effective therapeutic intervention may be provided by complementation with agonists of that dysfunctional WT-1-binding protein, as for example, by replacement gene therapy.

Where the nephroblastoma does not result from mutation of either WT1 or a WT1-binding protein, agents which alter the complexes formed by at least one of the subject WT1-binding proteins and WT1 (or other regulatory proteins), may nevertheless provide anti-neoplastic treatment regimens for tumor therapy.

In similar fashion, agents which modulate the function of the subject WT1-binding proteins can be used to treat malignant growth of other tissue in which events mediated by one or more of the subject WT1-binding proteins may contribute in some manner to neoplastic transformation. For instance, analogous to epithelial cells of the renal tube (which are transformed in Wilms tumor at the metanephronic blastema stage of development), mesothelial cells, e.g. epithelial cell layers which line serosal surfaces, undergo mesenchymal-epithelial transition during development. Moreover, there are histologically similar characteristics between mesotheliomas, the tumors which arise from mesothelium, and nephroblastomas, including the apparent involvement of WT1 (Walker et al. (1994) Cancer Res 54:3101-3106). Consequently, discovery of the interaction between WT1 and the subject WT1-binding proteins, as well as the concomitant realization of potential pharmaceutical agents derived with these proteins, provides possible therapeutic courses for intervention in the treatment of mesotheliomas. For example, contacting mesothelioma cells with an agent that modulates the function of a WT1-binding protein may, as in the case of Wilms tumor therapies, inhibit tumor cell growth by causing differentiation of tumorogenic cells to post-mitotic tissue and/or cell death.

Moreover, the expression of WT1 in other tissue, such as the bladder, testis and other urogenital tissue, as well as in lung, heart and spleen tissue, shows that corresponding modes of intervention may exist for neoplasias and hyperplasias originating from these tissues. For instance, in yet another exemplary embodiment, manipulating the involvement of WT1-binding proteins in transcriptional regulatory complexes of endometrial cells can provide a method of treating endometriosis.

Likewise, WT1 has been found to be expressed in hematopoietic cells, indicating a potential role for that protein in hematopoietic differentiation and regulation, as well as possible involvement in hematopoietic tumors.

In yet another embodiment, the subject method can be used in the treatment of neoplastic or hyperplastic transformations of the central nervous system. For instance, certain of the WT1-binding proteins are likely to be involved in regulation of neuronal cells, and therefore could be manipulated to cause re-differentiation or apoptosis of transformed neuronal cells. Treatment with agents which affect the function of the subject WT1-binding proteins in neuronal differentiation, may involve, for example, disruption of autocrine loops, e.g., TGF-b or PDGF autostimulatory loops, believed to be involved in the neoplastic transformation of several neuronal tumors. Accordingly, the subject method may be of use in the treatment of, for example, malignant gliomas, medulloblastomas, neuroectodermal tumors, and ependymonas.

Manipulation of the interaction between a WT1-binding protein and WT1, or other regulatory protein, may be useful for reshaping organs in vivo as well as in vitro organ and tissue cultures. In one embodiment, the present invention makes use of the apparent involvement of the subject WT1-binding proteins in controlling the development of stem cells responsible for formation of the kidneys, as well as of the pancreas, lungs and other organs which derive from the primitive gut. For example, therapeutic compositions for modulating the action of one or more WT1-binding proteins can be utilized in conjunction with transplantation of artificial organs, such as kidney structures, so as to promote implantation, vascularization, and in vivo differentiation and maintenance of the engrafted kidney tissue. In yet another embodiment, the subject method can be employed therapeutically to regulate such organs after physical, chemical or pathological insult. For instance, manipulation of WT1 function via control of the subject WT-1-binding proteins can be utilized in renal repair subsequent to, for example, ischemic injury to the kidney. For instance, regeneration of proximal tubule epithelium by the subject method can present opportunities to effectuate nephrogenic repair in vivo.

Similarly, therapeutic compositions targeting WT1-BP function may be useful to promote regeneration of lung tissue in the treatment of emphysema and other degenerative conditions of the lung. For example, the subject method may useful in the treatment of degenerative disorders of lung tissue caused by, for instance, toxic injuries, as well as inflammatory and degenerative processes induced by viral infections. Tissue degeneration of the lung which may be treatable by the present invention includes degenerative changes affecting the endothelial and epithelial cells, basal membrane, and mesenchymal and matrix structures.

In similar fashion, the subject method can be applied to enhancing both the generation of prosthetic tissue devices and to their implantation. Manipulation of WT1 function, by altering the ability of the protein to form complexes with the subject WT1-binding proteins, can provide a means for more carefully controlling the characteristics of a cultured tissue. In an exemplary embodiment, prosthetic devices which require a mesothelial lining can be generated by the subject method. For example, vascular grafts can be created utilizing vascular prostheses material (see, for example, Pronk et al. (1994) Cell Transplant 3:4148) seeded with human peritoneal mesothelial cells, as can prosthetic cardiac valves and prostheses for the repair of abdominal wall hernias. By manipulating the characteristics of the seeded cells by a method which includes either restricting or potentiating the function of certain of the subject WT1-binding proteins, medical devices can be designed which make use of desired cell types on a particular surface prior to grafting. In the aforementioned examples, mesothelial differentiation can be more faithfully controlled.

In yet another embodiment, the subject method may be used to enhance attachment of prosthetic devices. To illustrate, the subject method can be used in the implantation of luminal stents or urogenital prostheses, or other devices where it may be desirable to promote bonding of the surrounding tissue or to repair lesions to serosal epithelial layers. Manipulation of tissue surrounding a prosthesis can be used to integrate the prostheses, as well as to inhibit formation of fibrotic tissue proximate the prosthetic device.

Yet another aspect of the present invention concerns the therapeutic application of agents which either disrupt or potentiate the action of at least one of the subject WT1-binding proteins in order to enhance survival of neurons and other neuronal cells in both the central nervous system and the peripheral nervous system. The expression of WT1 in motor neurons, along with the observation that various neuropathies can accompany genetic lesions to the WT1 locus, is consistent with the ability of WT1 to regulate neuronal differentiation and survival during development of the nervous system and also, presumably, in the adult state, and hence at least some of the WT1-binding proteins of the present invention to modulate the regulation by WT1. Accordingly, affecting the formation of regulatory complexes containing the subject WT1-binding proteins can reasonably be expected to facilitate control of adult neurons with regard to maintenance, functional performance, and aging of normal cells; repair and regeneration processes in chemically or mechanically lesioned cells; and prevention of degeneration and premature death which result from loss of differentiation in certain pathological conditions. In light of this understanding, the present invention specifically contemplates applications of the subject method to the treatment of (prevention and/or reduction of the severity of) neurological conditions deriving from: (i) acute, subacute, or chronic injury to the nervous system, including traumatic injury, chemical injury, vasal injury and deficits (such as the ischemia resulting from stroke), together with infectious/inflammatory and tumor-induced injury; (ii) chronic neurodegenerative diseases of the nervous system, including Parkinson's disease, Huntington's chorea, amylotrophic lateral sclerosis and the like, as well as spinocerebellar degenerations; (iii) aging of the nervous system including Alzheimer's disease; and (iv) chronic immunological diseases of the nervous system or affecting the nervous system, including multiple sclerosis.

It is intended by the present invention that the subject method, involving treatment with agents which modulate the cellular function of one or more of the subject WT-1-binding proteins, can be used in the treatment of neurogenic and myopathic diseases which ultimately affect the somatic division of the peripheral nervous system and which are manifest as neuromuscular disorders. Examples include chronic atrophies such as amyotrophic lateral sclerosis, Guillain-Barre syndrome and chronic peripheral neuropathy, as well as other diseases which can be manifest as progressive bulbar palsies or spinal muscular atrophies.

For example, the subject method can be used to treat amyotrophic lateral sclerosis. ALS is a name given to a complex of disorders that comprise upper and lower motor neurons. Patients may present with progressive spinal muscular atrophy, progressive bulbar palsy, primary lateral sclerosis, or a combination of these conditions. The major pathological abnormality is characterized by a selective and progressive degeneration of the lower motor neurons in the spinal cord and the upper motor neurons in the cerebral cortex. Therapeutic agents which control the ability of WT-1-binding proteins to participate in regulatory complexes that influence apoptosis and/or differentiation, can be used alone or in conjunction with other neurotrophic factors such as CNTF, BDNF, NGF, hedgehog or noggin, to prevent and/or reverse motor neuron degeneration in ALS patients by enhancing motor neuron survival.

Furthermore, a potential role of WT1-BP modulators involves the development and maintenance of dendritic processes of axonal neurons. In particular, such therapeutic intervention may be useful in treatments designed to rescue, for example, motomeurons, from lesion-induced death as well as guiding reprojection of these neurons after such damage. Such diseases and conditions include but are not limited to CNS trauma, infarction, infection (such as viral infection with varicella-zoster), metabolic disease, nutritional deficiency, toxic agents (such as cisplatin treatment). For instance, control of motor neurons by the present method can be used in nerve prostheses for the repair of central and peripheral nerve damage. In particular, where a crushed or severed axon is entubulated by use of a prosthetic device, manipulation of WT1-BP activities in the cell can be used to increase the rate of growth and regeneration of the dendritic processes. Exemplary nerve guidance channels are described in U.S. Pat. Nos.5,092,871 and 4,955,892. Accordingly, a severed axonal process can be directed toward the nerve ending from which it was severed.

The subject method may also be applied opportunely in the treatment of neurodegenerative disorders which have manifestations of tremors and involuntary movements. Parkinson's disease, for example, primarily affects subcortical structures and is characterized by degeneration of the nigrostriatal pathway, raphe nuclei, locus ceruleus, and the motor nucleus of vagus. Ballism is typically associated with damage to the subthalmic nucleus, often due to acute vascular accident.

In yet another embodiment of the present invention, the subject method can be used to inhibit spermatogenesis. For instance, in light of the finding that WT1 and par-4 are apparently involved in the differentiation and/or proliferation and maintenance of testicular germ cells, antagonist of transcriptional regulatory complexes involving one or more of the subject WT1-binding proteins can be utilized to block the biological activity of WT1 and/or par-4 with respect to spermatogenesis, by competitvely inhibiting their function in the testis. In similar fashion, WT1-BP agonists and antagonists are potentially useful for modulating normal ovarian function.

As set out above, the present method is also applicable to cell culture techniques. In one embodiment, manipulation of differentiative states of renal or urogenital tissue can be performed in order to provide cell lines, especially primary cell lines, which maintain a particular phenotype. For example, modulating the action of WT1 via interactions with one or more of the subject WT1-binding proteins can be used to establish cell lines which are derived from uteric bud cells and maintain their phenotype in culture. In another embodiment, the differentiation of gondal tissue in culture, such as Sertoli cells, can be controlled by manipulation of the subject WT-1-binding proteins.

Furthermore, the subject method can be used to maintain neuronal cell cultures. For instance, in vitro neuronal culture systems have proved to be fundamental and indispensable tools for the study of neural development, as well as the identification of neurotrophic factors such as nerve growth factor (NGF), ciliary trophic factors (CNTF), and brain derived neurotrophic factor (BDNF). Once a neuronal cell has become terminally-differentiated it typically will not change to another terminally differentiated cell-type. However, neuronal cells can nevertheless readily lose their differentiated state. This is commonly observed when they are grown in culture from adult tissue, and when they form a blastema during regeneration. The present method provides a means for ensuring an adequately restrictive environment in order to maintain neuronal cells at various stages of differentiation, and can be employed, for instance, in cell cultures designed to test the specific activities of other trophic factors. In such embodiments of the subject method, cultured motor neuron cells, or progenitor cells which give rise to motor neurons, can be contacted with an agent which enhances the role of WT1 and WT-1-binding proteins in neuronal differentiation, such as a gene construct which causes over-expression of Ciao-1 or par-4, in order to induce neuronal differentiation (e.g. of a stem cell), or to maintain the integrity (e.g. promote survival) of a culture of terminally-differentiated neuronal cells by preventing loss of differentiation. In an exemplary embodiment, a naive neuronal cell is treated with an expression vector encoding an agonistic form of a WT-1-binding protein in order to induce differentiation of the cells into, for example, motor neurons.

Conversely, control of the function of one or more of the subject WT1-binding proteins can be accomplished to inhibit differentiation along WT-1-mediated pathways, particularly where uncommitted pluripotent stem cells are being cultured, so that cultures can be induced along one of an alternate developmental pathways. Accordingly, manipulation of WT1-binding protein function in cultured stem cells can be to induce differentiation of uncommitted progenitor cells in order to give rise to a committed progenitor cell, or to cause further restriction of the developmental fate of a committed progenitor cell along a path towards becoming a terminally-differentiated neuronal cell. Such neuronal cultures can be used as convenient assay systems as well as sources of implantable cells for therapeutic treatments.

The manipulation of the biological function of the subject WT1-binding proteins can be carried out solely using such reagents, as for example, described herein, or in combination with treatment with trophic and other growth factors which act to more particularly enhance a specific differentiation fate of the neuronal progenitor cell. In the later instance, manipulation of WT1-binding protein involvement in cell regulation might be viewed as ensuring that the treated cell is poised along a certain developmental pathway so as to be properly induced upon contact with a neurotrophic factor.

Another aspect of the present invention concerns recombinant forms of the subject WT1-binding proteins which are encoded by genes derived from eukaryotic organisms, e.g. mammals, e.g. humans, and which possess at least one biological activity of a naturally occurring form of the protein, or is an antagonist thereof (including naturally occurring dysfunctional mutants). The term “recombinant protein” refers to a protein of the present invention which is produced by recombinant DNA techniques, wherein generally DNA encoding the subject WT-1-binding protein is inserted into a suitable expression vector which is in turn used to transform a host cell to produce the heterologous protein. Moreover, the phrase “derived from”, with respect to a recombinant gene, is meant to include within the meaning of “recombinant protein” those proteins having an amino acid sequence of a native WT1-binding protein, or an amino acid sequence similar thereto which is generated by mutations including substitutions and deletions (including truncation). Recombinant proteins preferred by the present invention, in addition to native WT1-binding proteins, are at least 60% homologous, more preferably 70% homologous and most preferably 80% homologous with an amino acid sequence shown in one of SEQ ID Nos: 3 and 4. Polypeptides having an activity of the subject WT-1-binding proteins (i.e. either agonistic or antagonistic of the naturally-occurring form of the protein) and having at least about 90%, more preferably at least about 95%, and most preferably at least about 98-99% homology with a sequence of either in SEQ ID No: 3 and 4 are also within the scope of the invention.

The present invention further pertains to recombinant forms of the subject WT1-binding proteins which evolutionarily related to a WT-1-binding protein of represented in one of SEQ ID No: 3 and 4, that is, not identical, yet which are capable of functioning as an agonist or an antagonist of at least one biological activity of a WT1-binding protein. The term “evolutionarily related to”, with respect to amino acid sequences of recombinant WT1-binding proteins, refers to proteins which have amino acid sequences that have arisen naturally, as well as to mutational variants which are derived, for example, by recombinant mutagenesis. Such evolutionarily derived WT1-binding proteins preferred by the present invention are at least 60% homologous, more preferably 70% homologous and most preferably 80% homologous with an amino acid sequence shown in one of SEQ ID No: 3 and SEQ ID No: 4. Polypeptides having at least about 90%, more preferably at least about 95%, and most preferably at least about 98-99% homology with a sequence shown in any of SEQ ID Nos: 3 and 4 are also within the scope of the invention.

The present invention further pertains to methods of producing the subject WT1-binding proteins. For example, a host cell transfected with a nucleic acid vector directing expression of a WT1-binding protein can be cultured under appropriate conditions to allow expression of the polypeptide to occur. The polypeptide may be secreted, e.g. with the use of an exogenous signal sequence, and isolated from a mixture of cells and medium containing the recombinant WT1-BP. Alternatively, the peptide may be retained cytoplasmically, as the naturally occurring form of the protein is believed to be, and the cells harvested, lysed and the protein isolated. A cell culture includes host cells, media and other byproducts. Suitable media for cell culture are well known in the art. The recombinant WT1-binding protein can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant WT1-binding protein is a fusion protein containing a domain which facilitates its purification, such as a glutathione-S-transferase domain or a polyhistidine leader sequence in the form of a fusion protein with the subject polypeptides.

This invention also pertains to a host cell transfected with a WT1-BP gene to express a recombinant form of a WT1-binding protein. The host cell may be any prokaryotic or eukaryotic cell. Thus, a nucleotide sequence derived from the cloning of the WT1-binding proteins of the present invention, encoding all or a selected portion of a protein, can be used to produce a recombinant form of a WT1-BP via microbial or eukaryotic cellular processes. Ligating a polynucleotide sequence into a gene construct, such as an expression vector, and transforming or transfecting host cells with the vector are standard procedures used in producing other well-known proteins, e.g. insulin, interferons, p53, myc, cyclins and the like. Similar procedures, or modifications thereof, can be employed to prepare recombinant WT1-binding proteins, or portions thereof, by microbial means or tissue-culture technology in accord with the subject invention. Host cells suitable for expression of a recombinant WT1-binding protein can be selected, for example, from amongst eukaryotic (yeast, avian, insect or mammalian) or prokaryotic (bacterial) cells.

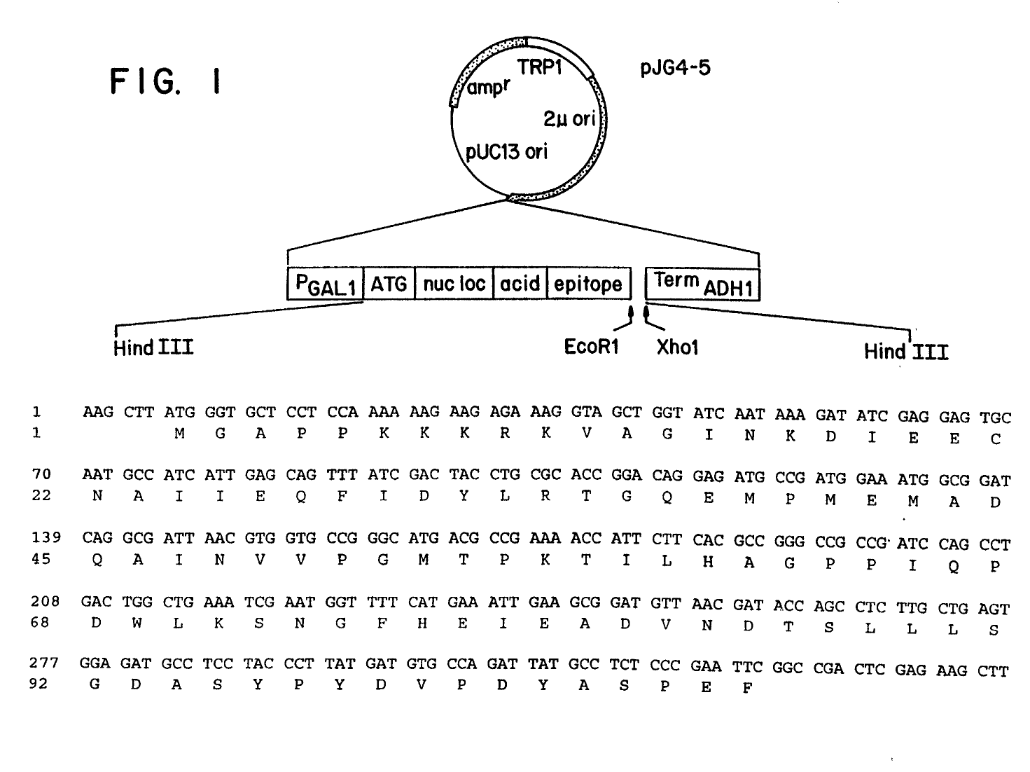

The recombinant WT1-BP gene can be produced by ligating nucleic acid encoding a WT1-binding protein, or a portion thereof, into a vector suitable for expression in either prokaryotic cells, eukaryotic cells, or both. Expression vectors for production of recombinant forms of WT-1-binding proteins include plasmids and other vectors. For instance, suitable vectors for the expression of a WT1-BP include plasmids of the types: pBR322-derived plasmids, pEMBL-derived plasmids, pEX-derived plasmids, pBTac-derived plasmids and pUC-derived plasmids for expression in prokaryotic cells, such as E. coli. In an illustrative embodiment, a WT1-binding protein is produced recombinantly utilizing an expression vector generated by sub-cloning a gene encoding the protein from the pSHI-WT1BP library, using, for example, primers based on SEQ ID No: 1 and 2 (see Table 3) and/or primers based on the flanking plasmid sequence (e.g. the primers represented by the sequence in FIG. 1).

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

Preferred mammalian expression vectors contain prokaryotic sequences to facilitate the propagation of the vector in bacteria, and one or more eukaryotic transcription regulatory sequences that cause expression of a recombinant WT1-BP gene in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found above in the description of gene therapy delivery systems.

In some instances, it may be desirable to express a recombinant WT1-binding protein by the use of a baculovirus expression system (see, for example, Current Protocols in Molecular Biology, eds. Ausubel et al. John Wiley & Sons: 1992). Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III).

The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press: 1989) Chapters 16 and 17.

When expression of a portion of one of the subject WT1-binding proteins is desired, i.e. a trunction mutant, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine, if desired, can be achieved either in vivo by expressing WT1-BP-derived polypeptides in a host which produces MAP (e.g., E. coli or CM89 or S. cerevisiae), or in vitro by use of purified MAP (e.g., procedure of Miller et al., supra).

Alternatively, the coding sequences for the polypeptide can be incorporated as a part of a fusion gene. This type of expression system can be useful under conditions where it is desirable to produce an immunogenic fragment of a WT1-binding protein. For example, the VP6 capsid protein of rotavirus can be used as an immunologic carrier protein for portions of the WT1-BP polypeptide, either in the monomeric form or in the form of a viral particle. The nucleic acid sequences corresponding to the portion of a subject WT1-binding protein to which antibodies are to be raised can be incorporated into a fusion gene construct which includes coding sequences for a late vaccinia virus structural protein to produce a set of recombinant viruses expressing fusion proteins comprising a portion of the protein WT1-BP as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of a WT-1-binding protein and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple Antigen Peptide system for peptide-based immunization can also be utilized to generate an immunogen, wherein a desired portion of a subject WT1-binding protein is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of the subject WT1-binding proteins can also be expressed and presented by bacterial cells.

In addition to utilizing fusion proteins to enhance immunogenicity, it is widely appreciated that fusion proteins can also facilitate the expression and purification of proteins, such as any one of the WT1-binding proteins of the present invention. For example, a WT1-binding protein can be generated as a glutathione-S-transferase (GST) fusion protein. Such GST fusion proteins can simplify purification of a WT l-binding protein, as for example by affinity purification using glutathione-derivatized matrices (see, for example, Current Protocols in Molecular Biology, eds. Ausubel et al. (N.Y.: John Wiley & Sons, 1991)). In another embodiment, a fusion gene coding for a purification leader sequence, such as a peptide leader sequence comprising a poly-(His)/enterokinase cleavage sequence, can be added to the N-terminus of the desired portion of a WT-1-binding protein in order to permit purification of the poly(His)-fusion protein by affinity chromatography using a Ni²⁺ metal resin. The purification leader sequence can then be subsequently removed by treatment with enterokinase (e.g., see Hochuli et al. (1987) J. Chromatography 411:177; and Janknecht et al. PNAS 88:8972).

Techniques for making fusion genes are known to those skilled in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which are subsequently annealed to generate a chimeric gene sequence (see, for example, Current Protocols in Molecular Biology, eds. Ausubel et al. John Wiley & Sons: 1992).

The present invention also makes available isolated WT1-binding polypeptides which are isolated from, or otherwise substantially free of other cellular proteins, especially WT1, p53 or other transcriptional regulatory factors, normally associated with the WT1-binding protein. The term “substantially free of other cellular or viral proteins” (also referred to herein as “contaminating proteins”) or “substantially pure or purified preparations” are defined as encompassing preparations of WT-BP polypeptides having less than 20% (by dry weight) contaminating protein, and preferably having less than 5% contaminating protein. Functional forms of the subject WT1-binding proteins can be prepared, for the first time, as purified preparations by using recombinant proteins as described herein. Alternatively, the subject WT1-binding proteins can be isolated by affinity purification using, for example, matrix bound WT1 protein. By “purified”, it is meant, when referring to a peptide or DNA or RNA sequence, that the indicated molecule is present in the substantial absence of other biological macromolecules, such as other proteins (particularly transcriptional factors such as WT1, as well as other contaminating proteins). The term “purified” as used herein preferably means at least 80% by dry weight, more preferably in the range of 95-99% by weight, and most preferably at least 99% by weight, of biological macromolecules of the same type present (but water, buffers, and other small molecules, especially molecules having a molecular weight of less than 5000, can be present). The term “pure” as used herein preferably has the same numerical limits as “purified” immediately above. “Isolated” and “purified” do not encompass either natural materials in their native state or natural materials that have been separated into components (e.g., in an acrylamide gel) but not obtained either as pure (e.g. lacking contaminating proteins, or chromatography reagents such as denaturing agents and polymers, e.g. acrylamide or agarose) substances or solutions.

Furthermore, isolated peptidyl portions of the subject WT1-binding proteins can also be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a WT1-binding protein of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a WT1-binding protein activity, such as by microinjection assays or in vitro protein binding assays. In an illustrative embodiment, peptidyl portions of a WT1-binding protein, such as Ciao-1 or par-4, can tested for WT1-binding activity by expression as thioredoxin fusion proteins, each of which contains a discrete fragment of the WT1-binding protein (see, for example, U.S. Pat. Nos. 5,270,181 and 5,292,646; and PCT publication WO94/02502).

It will also be possible to modify the structure of a WT1-binding protein for such purposes as enhancing therapeutic or prophylactic efficacy, or stability (e.g., ex vivo shelf life and resistance to proteolytic degradation in vivo). Such modified peptides, when designed to retain at least one activity of the naturally-occurring form of the protein, are considered functional equivalents of the WT1-binding protein described in more detail herein. Such modified peptide can be produced, for instance, by amino acid substitution, deletion, or addition.

For example, it is reasonable to expect that an isolated replacement of a leucine with an isoleucine or valine, an aspartate with a glutamate, a threonine with a serine, or a similar replacement of an amino acid with a structurally related amino acid (i.e. conservative mutations) will not have a major effect on the folding of the protein, and may or may not have much of an effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine (see, for example, Biochemistry, 2nd ed., Ed. by L. Stryer, W H Freeman and Co.: 1981). Alternatively, amino acid replacement can be based on steric criteria, e.g. isosteric replacements, without regard for polarity or charge of amino acid sidechains. Whether a change in the amino acid sequence of a peptide results in a functional WT1-BP homolog (e.g. functional in the sense that it acts to mimic or antagonize the wild-type form) can be readily determined by assessing the ability of the variant peptide to produce a response in cells in a fashion similar to the wild-type WT1-BP or competitively inhibit such a response. Peptides in which more than one replacement has taken place can readily be tested in the same manner.

This invention further contemplates a method of generating sets of combinatorial mutants of the presently disclosed WT1-binding proteins, as well as truncation and fragmentation mutants, and is especially useful for identifying potential variant sequences which are functional in binding to a WT1 protein but differ from a wild-type form of the protein by, for example, efficacy, potency and/or intracellular half-life. One purpose for screening such combinatorial libraries is, for example, to isolate novel WT1-BP homologs which function as either an agonist or an antagonist of the biological activities of the wild-type protein, or alternatively, possess novel activities all together. To illustrate, WT1-BP homologs can be engineered by the present method to provide proteins which bind WT1 yet prevent complete assembly of WT1-dependent transcription regulatory complexes. Such proteins, when expressed from recombinant DNA constructs, can be used in gene therapy protocols.

Likewise, mutagenesis can give rise to WT1-BP homologs which have intracellular half-lives dramatically different than the corresponding wild-type protein. For example, the altered protein can be rendered either more stable or less stable to proteolytic degradation or other cellular process which result in destruction of, or otherwise inactivation of, a WT1-binding protein. Such WT1-BP homologs, and the genes which encode them, can be utilized to alter the envelope of expression for a particular recombinant WT1-binding protein by modulating the half-life of the recombinant protein. For instance, a short half-life can give rise to more transient biological effects associated with a particular recombinant WT1-binding protein and, when part of an inducible expression system, can allow tighter control of recombinant protein levels within a cell. As above, such proteins, and particularly their recombinant nucleic acid constructs, can be used in gene therapy protocols.

In an illustrative embodiment of this method, the amino acid sequences for a population of WT1-BP homologs or other related proteins are aligned, preferably to promote the highest homology possible. Such a population of variants can include, for example, WT1-BP homologs from one or more species, or WT1-BP homologs from the same species but which differ due to mutation. Amino acids which appear at each position of the aligned sequences are selected to create a degenerate set of combinatorial sequences. There are many ways by which the library of potential WT1-BP homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential WT1-BP sequences. The synthesis of degenerate oligonucleotides is well known in the art (see for example, Narang, SA (1983) Tetrahedron 39:3; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos: 5,223,409, 5,198,346, and 5,096,815).

Alternatively, other forms of mutagenesis can be utilized to generate a combinatorial library, particularly where no other naturally occurring homologs have yet been sequenced. For example, WT1-BP homologs (both agonist and antagonist forms) can be generated and isolated from a library by screening using, for example, alanine scanning mutagenesis and the like (Ruf et al. (1994) Biochemistry 33:1565-1572; Wang et. al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085), by linker scanning mutagenesis (Gustin et al. (1993) Virology 193:653-660; Brown et al. (1992) Mol. Cell Biol. 12:2644-2652; McKnight et al. (1982) Science 232:316); by saturation mutagenesis (Meyers et al. (1986) Science 232:613); by PCR mutagenesis (Leung et al. (1989) Method Cell Mol Biol 1:11- 19); or by random mutagenesis (Miller et al. (1992) A Short Course in Bacterial Genetics, CSHL Press, Cold Spring Harbor, N.Y.; and Greener et al. (1994) Strategies in Mol Biol 7:32-34).

A wide range of techniques are known in the art for screening gene products of combinatorial libraries made by point mutations, as well as for screening cDNA libraries for gene products having a certain property. Such techniques will be generally adaptable for rapid screening of the gene libraries generated by the combinatorial mutagenesis of WT1-binding proteins. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate WT1-BP sequences created by combinatorial mutagenesis techniques.

In one screening assay, the candidate gene products are displayed on the surface of a cell or viral particle, and the ability of particular cells or viral particles to bind a WT1 protein via this gene product is detected in a “panning assay”. For instance, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). In a similar fashion, fluorescently labeled WT1 can be used to score for potentially functional WT1-BP homologs. Cells can be visually inspected and separated under a fluorescence microscope, or, where the morphology of the cell permits, separated by a fluorescence-activated cell sorter.

In an alternate embodiment, the gene library is expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (Ladner et al. PCT publication WO 90/02909; Garrard et al., PCT publication WO 92/09690; Marks et al. (1992) J. Biol. Chem. 267:16007-16010; Griffiths et al. (1993) EMBO J 12:725-734; Clackson et al. (1991) Nature 352:624-628; and Barbas et al. (1992) PNAS 89:4457-4461). In an illustrative embodiment, the recombinant phage antibody system (RPAS, Pharmacia Catalog number 27-9400-01) can be easily modified for use in expressing and WT1-BP combinatorial libraries, and the WT1-BP phage library can be panned on glutathione immobilized WT1-GST fusion proteins. Successive rounds of phage amplification and panning can greatly enrich for WT1-BP homologs which retain an ability to bind WT1and which can subsequently be screened further for biological activities in automated assays, in order to distinguish between agonists and antagonists.

The invention also provides for identification and reduction to functional minimal size of the WT1-binding domains of the subject WT1-binding proteins to generate mimetics, e.g. peptide or non-peptide agents, which are able to disrupt binding of a polypeptide of the present invention with a WT1 protein. Thus, such mutagenic techniques as described above are also useful to map the determinants of WT1-binding proteins which participate in protein-protein interactions involved in, for example, binding to a WT1 protein. To illustrate, the critical residues of a WT1-binding protein which are involved in molecular recognition of WT1 can be determined and used to generate WT1-BP-derived peptidomimetics that competitively inhibit binding of the WT1-BP to WT1. By employing, for example, scanning mutagenesis to map the amino acid residues of a particular WT1-binding protein involved in binding WT1, peptidomimetic compounds can be generated which mimic those residues in binding to WT1, and which, by inhibiting binding of the WT1-BP to WT1, can interfere with the function of WT1 in transcriptional regulation of one or more genes. For instance, non-hydrolyzable peptide analogs of such residues can be generated using retro-inverse peptides (e.g., see U.S. Pat. Nos. 5,116,947 and 5,218,089; and Pallai et al. (1983) Int J Pept Protein Res 21:84-92), benzodiazepine (e.g., see Freidinger et al. in Peptides: Chemistry and Biology, G. R. Marshall ed., ESCOM Publisher: Leiden, Netherlands, 1988), azepine (e.g., see Huffinan et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun126:419; and Dann et al. (1986) Biochem Biophys Res Commun 134:71).

Another aspect of the invention pertains to an antibody specifically reactive with one of the subject WT1-binding proteins. For example, by using immunogens derived from a WT1-binding protein, anti-protein/anti-peptide antisera or monoclonal antibodies can be made by standard protocols (See, for example, Antibodies: A Laboratory Manual Eds. Harlow and Lane (Cold Spring Harbor Press: 1988)). A mammal, such as a mouse, a hamster or rabbit can be immunized with an immunogenic form of the peptide (e.g., a full length WT1-binding protein or an antigenic fragment which is capable of eliciting an antibody response). Techniques for conferring immunogenicity on a protein or peptide include conjugation to carriers or other techniques well known in the art. An immunogenic portion of the subject WT1-binding proteins can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of the WT-1-binding proteins of the present invention, e.g. antigenic determinants of a protein represented in one of SEQ ID Nos: 3 and 4 or a closely related human or non-human mammalian homolog thereof. For instance, a favored anti-WT1-BP antibody of the present invention does not substantially cross react (i.e. react specifically) with a protein which is less than 90 percent homologous to one of SEQ ID Nos: 3 and 4; though antibodies which do not substantially cross react with a protein which is less than 95 percent homologous with one of SEQ ID Nos: 3 and 4, or even less than 98-99 percent homologous with one of SEQ ID Nos: 3 and 4, are specifically contemplated. By “not substantially cross react”, it is meant that the antibody has a binding affinity for a non-homologous protein (e.g. WT1) which is less than 10 percent, more preferably less than 5 percent, and even more preferably less than 1 percent, of the binding affinity for a protein represented one of SEQ ID Nos: 3 and 4.

Following immunization, anti-WT1-BP antisera can be obtained and, if desired, polyclonal anti-WT1-BP antibodies isolated from the serum. To produce monoclonal antibodies, antibody producing cells (lymphocytes) can be harvested from an immunized animal and fused by standard somatic cell fusion procedures with immortalizing cells such as myeloma cells to yield hybridoma cells. Such techniques are well known in the art, an include, for example, the hybridoma technique (originally developed by Kohler and Milstein, (1975) Nature, 256: 495497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a WT1-binding protein of the present invention and monoclonal antibodies isolated from a culture comprising such hybridoma cells.

The term antibody as used herein is intended to include fragments thereof which are also specifically reactive with one of the subject WT1-binding protein. Antibodies can be fragmented using conventional techniques, including recombinant engineering, and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. The antibody of the present invention is further. intended to include bispecific and chimeric molecules having an anti-WT1-BP portion.

Both monoclonal and polyclonal antibodies (Ab) directed against a WT1-binding protein can be used to block the action of that protein and allow the study of the role of a particular WT1-binding protein in transcriptional regulation generally, or in the etiology of Wilm's tumors specifically, e.g. by microinjection of anti-WT1-BP into cells.

Antibodies which specifically bind WT1-BP epitopes can also be used in immunohistochemical staining of tissue samples in order to evaluate the abundance and pattern of expression of each of the subject WT1-binding proteins. Anti-WT-1-BP antibodies can be used diagnostically in immuno-precipitation and immuno-blotting to detect and evaluate WT1-BP levels in tissue or bodily fluid as part of a clinical testing procedure. For instance, such measurements as the level of WT1-BP/WT1 complexes can be useful in predictive valuations of the onset or progression of, for example, nephroblastomas such as Wilm's tumors. Likewise, the ability to monitor WT1-BP levels in the cells of an individual can permit determination of the efficacy of a given treatment regimen for an individual afflicted with such a disorder. The level of a WT1-binding protein can be measured in cells found in bodily fluid, such as in samples of cerebral spinal fluid, or can be measured in tissue, such as produced by biopsy. Diagnostic assays using anti-WT-1-BP antibodies can include, for example, immunoassays designed to aid in early diagnosis of a neoplastic or hyperplastic disorder, and may aid in detecting the presence of cancerous cells in the sample, e.g. Wilm's tumor cells, by detecting cells in which a lesion of the WT1-BP gene has occurred or in which the protein is misexpressed or found in abnormal protein complexes.

Another application of the subject antibodies is in the immunological screening of cDNA libraries constructed in expression vectors such as λgt11, λgt18-23, λZAP, and λORF8. Messenger libraries of this type, having coding sequences inserted in the correct reading frame and orientation, can produce fusion proteins. For instance, λgt11 will produce fusion proteins whose amino termini consist of β-galactosidase amino acid sequences and whose carboxy termini consist of a foreign polypeptide. Antigenic epitopes of a WT1-binding protein can then be detected with antibodies, as, for example, reacting nitrocellulose filters lifted from infected plates with anti-WT1-BP antibodies. Phage, scored by this assay, can then be isolated from the infected plate. Thus, the presence of WT1-BP homologs can be detected and cloned from other animals, and alternate isoforms (including splicing variants) can be detected and cloned from human sources.

Moreover, the nucleotide sequence determined from the clones of the subject WT1-binding proteins from a human cell line will allow generation of further probes to identify homologs in other human cell types, and WT1-BP homologs (e.g. orthologs) from other animals. For instance, the present invention also provides a probe/primer comprising a substantially purified oligonucleotide, which oligonucleotide comprises a region of nucleotide sequence that hybridizes under stringent conditions to at least 10 consecutive nucleotides of sense or anti-sense sequence of one of SEQ ID Nos: 1 and 2, or naturally occurring mutants thereof. In preferred embodiments, the probe/primer further comprises a label group attached thereto and able to be detected, e.g. the label group is selected from the group consisting of radioisotopes, fluorescent compounds, enzymes, and enzyme co-factors. Such probes can also be used as a part of a diagnostic test kit for identifying transformed cells, such as for measuring a level of a WT1-BP nucleic acid in a sample of cells from a patient; e.g. detecting mRNA encoding a WT1-BP mRNA level; e.g. determining whether a genomic WT1-BP gene has been mutated by transition, transversion, inversion, deletion, duplication, or transposition. A diagnostic for duplication of a WT1-BP on chromosome IV, for example, encoding the par-4 gene, may be useful for prediction of cell transformation, associated with potential for a predisposition for cancer.

In addition, nucleotide probes can be generated which allow for histological screening of intact tissue and tissue samples for the presence of a WT1-BP mRNA. Similar to the diagnostic uses of anti-WT1-BP antibodies, the use of probes directed to WT1-BP mRNAs, or to genomic WT1-BP sequences, can be used for both predictive and therapeutic evaluation of allelic mutations which might be manifest in, for example, neoplastic or hyperplastic disorders (e.g. unwanted cell growth) or abnormal differentiation of tissue. The nucleotide probes can facilitate determination of the presence of a mutation as the basis for the presence of a developmental disorder, because the mutuation causes an abnormality associated with activity (or lack thereof) or expression (or lack thereof) of a WT1-binding protein. For instance, variation in synthesis of a WT1-binding protein can be distinguished from a mutation in the genes coding sequence.

Accordingly, the present method provides a method for determining if a subject is at risk for a disorder characterized by unwanted cell proliferation or generally aberrant control of differentiation. In preferred embodiments, the subject method can be characterized generally comprising detecting, in a tissue sample of the subject (e.g. a human patient), the presence or absence of a genetic lesion characterized by at least one of (i) a mutation of a gene encoding one of the subject WT-1-binding proteins or (ii) the mis-expression of a WT1-BP gene. To illustrate, such genetic lesions can be detected by ascertaining the existence of at least one of (i) a deletion of one or more nucleotides from a WT1-BP gene, (ii) an addition of one or more nucleotides to such a WT1-BP gene, (iii) a substitution of one or more nucleotides of a WT1-BP gene, (iv) a gross chromosomal rearrangement or amplification of a WT1-BP gene, (v) a gross alteration in the level of a messenger RNA transcript of a WT1-BP gene, (vi) aberrant modification of a WT1-BP gene, such as of the methylation pattern of the genomic DNA, (vii) the presence of a non-wild type splicing pattern of a messenger RNA transcript of a WT1-BP gene, and (viii) a non-wild type level of a WT1-binding protein. As set out below, the present invention provides a large number of assay techniques for detecting lesions in a WT1-BP-gene, and importantly, provides the ability to discern between different molecular causes underlying WT1-BP-dependent aberrant cell growth, proliferation and/or differentiation.

In an exemplary embodiment, there is provided a nucleic acid composition comprising a (purified) oligonucleotide probe including a region of nucleotide sequence which is capable of hybridizing to a sense or antisense sequence of a WT1-BP-gene, such as represented by any of SEQ ID Nos: 1 or 2, or naturally occurring mutants thereof, or 5′ or 3′ flanking sequences or intronic sequences naturally associated with the subject WT1-BP genes or naturally occurring mutants thereof. The nucleic acid of a cell is rendered accessible for hybridization, the probe is exposed to nucleic acid of the sample, and the hybridization of the probe to the sample nucleic acid is detected. Such techniques can be used to detect lesions at either the genomic or mRNA level, including deletions, substitutions, etc., as well as to determine mRNA transcript levels.

In certain embodiments, detection of the lesion comprises utilizing the probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080), which can be particularly useful for detecting point mutations in the WT1-BP gene. In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize to a WT1-BP gene under conditions such that hybridization and amplification of the WT1-BP gene (if present) occurs, and (iv) detecting the presence or absence of an amplification product, or detecting the size of the amplification product and comparing the length to a control sample.

In still another embodiment, the level of a WT1-BP protein can be detected by immunoassay. For instance, the cells of a biopsy sample can be lysed, and the level of a WT1-BP protein present in the cell can be quantitated by standard immunoassay techniques.

In yet another exemplary embodiment, aberrant methylation patterns of a WT1-BP gene can be detected by digesting genomic DNA from a patient sample with one or more restriction endonucleases that are sensitive to methylation and for which recognition sites exist in the WT1-BP gene (including in the flanking and intronic sequences). See, for example, Buiting et al. (1994) Human Mol Genet 3:893-895. Digested DNA is separated by gel electrophoresis, and hybridized with probes derived from, for example, genomic or cDNA sequences. The methylation status of the WT1-BP gene can be determined by comparison of the restriction pattern generated from the sample DNA with that for a standard of known methylation.

Furthermore, the subject gene constructs described above can be utilized in diagnostic assays to determine if a cell's growth or differentiation state is no longer dependent on the regulatory function of a WT1-BP protein, e.g. in determining the phenotype of a transformed cell. Such knowledge can have both prognostic and therapeutic benifits. Such knowledge can have both prognostic and therapeutic benefits. To illustrate, a sample of cells from the tissue can be obtained from a patient and dispersed in appropriate cell culture media, a portion of the cells in the sample can be caused to express a recombinant WT1-BP protein, e.g. by transfection with a ciao-1 or par-4 expression vector, and subsequent growth of the cells assessed. The absence of a change in phenotype of the cells despite expression of the WT1-BP protein may be indicative of a lack of dependence on cell regulatory pathways which include the WT1-BP protein, e.g. WT1-mediated transcription. Depending on the nature of the tissue of interest, the sample can be in the form of cells isolated from, for example, a blood sample, an exfoliated cell sample, a fine needle aspirant sample, or a biopsied tissue sample. Where the initial sample is a solid mass, the tissue sample can be minced or otherwise dispersed so that cells can be cultured, as is known in the art.

In yet another embodiment, a diagnostic assay is provided which detects the ability of a WT1-BP gene product, e.g., isolated from a biopsied cell, to bind to other cellular proteins. For instance, it will be desirable to detect WT1-BP mutants which, while expressed at appreciable levels in the cell, are defective at binding WT1 (having either diminished or enhanced binding affinity). Such mutants may arise, for example, from mutations, e.g., point mutants, which may be impractical to detect by the diagnostic DNA sequencing techniques or by the immunoassays described above. The present invention accordingly further contemplates diagnostic screening assays which generally comprise cloning one or more WT1-BP genes from the sample cells, and expressing the cloned genes under conditions which permit detection of an interaction between that recombinant gene product and a target protein, e.g., a WT1.

As will be apparent from the description of the various drug screening assays set forth below, a wide variety of techniques can be used to determine the ability of a WT1-BP protein to bind to other cellular components. These techniques can be used to detect mutations in a WT-1-BP gene which give rise to mutant proteins with a higher or lower binding affinity for a WT1 relative to the wild-type WT1-BP. Conversely, by switching which of the WT1 and WT1-BP protein is the “bait” and which is derived from the patient sample, the subject assay can also be used to detect WT1 mutants which have a higher or lower binding affinity for a WT-1-BP protein relative to a wild-type form of that WT1.

In an exemplary embodiment, WT1 (e.g. wild-type) can be provided as an immobilized protein (a “target”), such as by use of GST fusion proteins and glutathione-treated microtitre plates. A WT1-BP gene (a “sample” gene) is amplified from cells of a patient sample, e.g., by PCR, ligated into an expression vector, and transformed into an appropriate host cell. The recombinantly produced WT1-BP protein is then contacted with the immobilized WT1, e.g., as a lysate or a semi-purified preparation (see infra), the complex washed, and the amount of WT1/WT1-BP complex determined and compared to a level of wild-type complex formed in a control. Detection can be by, for instance, an immunoassay using antibodies against the wild-type form of the WT1-BP protein, or by virtue of a label provided by cloning the sample WT1-BP gene into a vector which provides the protein as a fusion protein including a detectable tag. For example, a myc epitope can be provided as part of a fusion protein with the sample WT1-BP gene. Such fusion proteins can, in addition to providing a detectable label, also permit purification of the sample WT1-BP protein from the lysate prior to application to the immobilized target.

In yet another embodiment of the subject screening assay, the two hybrid assay, described in the appended examples, can be used to detect mutations in either a WT1-BP gene or WT1 gene which alter complex formation between those two proteins. Accordingly, the present invention provides a convenient method for detecting mutants of WT1-BP genes encoding proteins which are unable to physically interact with a WT1 “bait” protein, which method relies on detecting the reconstitution of a transcriptional activator in a WT1-BP/WT1-dependent fashion.

The role of each of the subject WT1-BP in growth and differentiative events, such as those giving rise to Wilm's tumor, as well as normal cellular functions of each of the subject WT1-binding proteins, e.g. in regulation of transcription, can be investigated by inhibiting endogenous production of a particular WT-1-binding protein by anti-sense techniques (e.g. microinjection of antisense molecules, or transfection with plasmids whose transcripts are anti-sense with regard to a WT1-BP mRNA or gene sequence). Such techniques can be utilized in cell culture, and can also be used following the engineering of transgenic animals.

Furthermore, by making available purified and recombinant WT1-binding proteins, the present invention facilitates the development of assays which can be used to screen for drugs which are either agonists or antagonists of the cellular function of each of the subject WT1-binding proteins, or of their role in the pathogenesis of proliferative and differentiative disorders. For instance, an assay can be generated according to the present invention which evaluates the ability of a compound to modulate binding of a WT1-binding protein to a WT1 protein. A variety of assay formats will suffice and, in light of the present inventions, will be comprehended by one of ordinary skill in the art.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they are used to permit rapid screen of a large number of test compounds, with relatively easy detection of an alteration in a molecular target when contacted with a test compound. Moreover, the effects of cellular toxicity and/or bioavailability of the test compound do not affect the in vitro system, the assay instead being focused primarily on the effect of the drug on the molecular target as may be manifest in an alteration of binding affinity with other proteins or change in enzymatic properties of the molecular target. Accordingly, in an exemplary screening assay of the present invention, the compound of interest is contacted with a mixture generated from an isolated and purified WT1-binding protein, such as Ciao-1 or par-4, and a WT1 protein. Protein-protein WT1 interactions as a target for inhibition are also specifically contemplated as an embodiment of the subject assay. Detection and quantification of WT1/WT1-BP complexes provides a means for determining the compound's efficacy at inhibiting (or potentiating) complex formation between the WT1 protein and the WT1-binding protein. The efficacy of the test compound can be assessed by generating dose response curves from data obtained using various concentrations of the test compound. Moreover, a control assay can also be performed to provide a baseline for comparison. In the control assay, isolated and purified WT1-BP is added to a composition containing the WT1 protein, and the formation of WT1/WT1-BP complex is quantitated in the absence of the test compound.

Complex formation between the WT1-binding protein and a WT1 may be detected by a variety of techniques. For instance, modulation in the formation of complexes can be quantitated using, for example, detectably labelled proteins (e.g. radiolabelled, fluorescently labelled, or enzymatically labelled), by immunoassay, or by chromatographic detection.

Typically, it will be desirable to immobilize either the WT1 protein or the WT1-binding protein to facilitate separation of WT1/WT-1-BP complexes from uncomplexed forms of one of the proteins, as well as to accommodate automation of the assay. In an illustrative embodiment, a fusion protein can be provided which adds a domain that permits the protein to be bound to an insoluble matrix. For example, glutathione-S-transferase-WT1 (GST-WT1) fusion proteins can be adsorbed onto glutathione Sepharose beads (Sigma Chemical, St. Louis, Mo.) or glutathione derivatized microtitre plates, which are then combined with the WT-1-binding protein, e.g. an ³⁵S-labeled WT1-binding protein, and the test compound and incubated under conditions conducive to complex formation. Following incubation, the beads are washed to remove any unbound WT1-BP, and the matrix bead-bound radiolabel determined directly (e.g. beads placed in scintillant), or in the superntantant after the WT1/WT1-BP complexes are dissociated, e.g. when microtitre plates is used. Alternatively, after washing away unbound protein, the complexes can be dissociated from the matrix, separated by SDS-PAGE gel, and the level of WT1-BP found in the matrix-bound fraction quantitated from the gel using standard electrophoretic techniques.

Other techniques for immobilizing proteins on matrices are also available for use in the subject assay. For instance, the WT1 protein can be immobilized utilizing conjugation of biotin and streptavidin. For instance, biotinylated WT1 can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well microtiter plates (Pierce Chemical). Alternatively, antibodies reactive with WT1 can be derivatized to the wells of the plate, and WT1 trapped in the wells by antibody conjugation. As above, preparations of a WT1-binding protein and samples of test compounds are incubated in the WT1-presenting wells of the plate, and the amount of WT1/WT1-BP complex trapped in the well can be quantitated. Exemplary methods for detecting such complexes, in addition to those described above for the GST-immobilized complexes, include immunodetection of complexes using antibodies specific for the WT1-binding protein, or which are specific for the WT1 protein and compete for binding with the WT1-BP; as well as enzyme-linked assays which rely on detecting an enzymatic activity associated with the WT1-binding protein. In the instance of the latter, the enzyme can be chemically conjugated or provided as a fusion protein with the WT-1-binding protein. To illustrate, the WT1-binding protein can be chemically cross-linked with alkaline phosphatase, and the amount of WT1-BP trapped in the complex can be assessed with a chromogenic substrate of the enzyme, e.g. paranitrophenylphosphate. Likewise, a fusion protein comprising the WT1-BP and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the protein, such as the anti-WT1-BP antibodies described herein, can be used. Alternatively, the protein to be detected in the complex can be “epitope tagged” in the form of a fusion protein which includes, in addition to the WT1-BP or WT1 sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which include a 10-residue sequence from c-myc, as well as the pFLAG system (International Biotechnologies, Inc.) or the pEZZ-protein A system (Pharamacia, N.J.).

Additionally, the subject WT1-binding proteins can be used to generate an interaction trap assay, as described in the examples below (see also, U.S. Pat. No.: 5,283,317; PCT publication WO94/10300; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for detecting agents which either potentiate or attenuate complex formation between a WT1-binding protein and WT1. As described below, the interaction trap assay relies on reconstituting in vivo a functional transcriptional activator protein from two separate fusion proteins, one of which comprises the DNA-binding domain of a transcriptional activator fused to a WT1 protein. The second fusion protein comprises a transcriptional activation domain (e.g. able to initiate RNA polymerase binding and transcription) fused to one of the subject WT1-binding proteins. When the WT1 and WT1-binding protein interact, the two domains of the transcriptional activator protein are brought into sufficient proximity as to cause transcription of a reporter gene.

In addition to the LexA interaction trap described in the examples below, yet another illustrative embodiment comprises Saccharomyces cerevisiae YPB2 cells transformed simultaneously with a plasmid encoding a GAL4db-WT1 fusion (db: DNA binding domain) and with a plasmid encoding the GAL4 activation domain (GAL4ad) fused to a subject WT1-BP. Moreover, the strain is transformed such that the GAL4-responsive promoter drives expression of a phenotypic marker. For example, the ability to grow in the absence of histidine can depend on the expression of the HIS3 gene. When the HIS3 gene is placed under the control of a GAL4-responsive promoter, relief of the histidine auxotrophic phenotype indicates that a functional GAL4 activator has been reconstituted through the interaction of the WT1 and the WT1-BP proteins. Thus, an agent able to inhibit WT1-BP interaction with WT1 will result in yeast cells unable to grow on a defined medium in the absence of histidine. Alternatively, the phenotypic marker (e.g. instead of the HIS3 gene) can be one which provides a negative selection when expressed such that agents which disrupt WT1/WT1-BP interactions confer positive growth selection to the cells. Commercial kits which can be modified to develop two-hybrid assays with the subject WT1-binding proteins are presently available (e.g., MATCHMAKER kit, ClonTech catalog number K1605-1, Palo Alto, Calif.).

Moreover, in instances wherein one of the subject WT-1-binding proteins possesses an enzymatic activity, inhibitors of the enzymatic activity can be identified using assays derived from measuring the ability of an agent to inhibit catalytic conversion of a substrate by the subject enzyme.

Another aspect of the present invention concerns transgenic animals which are comprised of cells (of that animal) which contain a transgene of the present invention and which preferably (though optionally) express an exogenous WT1-binding protein in one or more cells in the animal. The WT1-BP transgene can encode the wild-type form of the protein, or can encode homologs thereof, including both agonists and antagonists, as well as antisense constructs designed to inhibit expression of the endogenous gene. In preferred embodiments, the expression of the transgene is restricted to specific subsets of cells, tissues or developmental stages utilizing, for example, cis-acting sequences that control expression in the desired pattern. In the present invention, such mosaic expression of the subject WT1-binding proteins can be useful for lineage analysis, and can additionally provide a means to assess the effects of, for example, lack of binding of a WT1-BP to WT1, which deficiency might grossly alter development in small patches of tissue within an otherwise normal embryo. Toward this end, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques which allow for the expression of transgenes can be regulated via site-specific in vivo genetic manipulation known to those of ordinary skill in the art. For instance, genetic systems are available which allow for the regulated expression of a recombinase that catalyzes the genetic recombination a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of a subject WT-1-binding protein. For example, excision of a target sequence which interferes with the expression of a recombinant WT1-BP gene can be designed to activate expression of that gene. This interference with expression of the gene by the target sequence can result from a variety of mechanisms, such as spatial separation of the gene from a promoter element or the presence of an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in the correct orientation with respect to the promoter element, which allows for promoter driven transcriptional activation.

In an illustrative embodiment, either the cre/loxP recombinase system of bacteriophage PI (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences, which are 34 base pair nucleotide repeat sequences to which the Cre recombinase binds and catalyzes Cre recombinase mediated genetic recombination. The orientation of loxP sequences determines whether the intervening target sequence is excised or inverted when Cre recombinase is present (Abremski et al. (1984) J. Biol. Chem. 259:1509-1514).

Accordingly, genetic recombination of the target sequence is made dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a WT1-binding protein can be regulated via regulation of recombinase expression.

Use of the cre/loxP recombinase system to regulate expression of a recombinant WT1-binding protein, such as Ciao-1 or par-4, requires the construction of a transgenic animal containing transgenes encoding each of the Cre recombinase and the subject protein. Animals containing genes both for the Cre recombinase and the recombinant WT1-BP can be provided, for example, by mating two transgenic animals each containing one transgene, e.g., mating the animal carrying the WT1-BP gene with the animal carrying the recombinase gene.

One advantage derived from initially constructing transgenic animals containing a transgene in a recombinase-mediated expressible format derives from the likelihood that the subject protein will be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues. Thus, the creation of a founder population in which, for example, an antagonistic WT1-BP transgene is silent will allow the study of progeny from that founder in which disruption of WT1 transcriptional regulatory complexes in a particular tissue or at developmental stages would result in, for example, a lethal phenotype.

Similar conditional transgenes can be provided using prokaryotic promoter sequences which require prokaryotic proteins to be simultaneous expressed in order to facilitate expression of the transgene. Exemplary promoters and the corresponding trans-activating prokaryotic proteins are given in U.S. Pat. No. 4,833,080. Moreover, expression of the conditional transgenes can be induced by gene therapy-like methods wherein a gene encoding the trans-activating protein, e.g. a recombinase or a prokaryotic protein, is delivered to the tissue and caused to be expressed using, for example, one of the gene therapy constructs described above. By this method, the WT1-BP transgene could remain silent into adulthood and its expression “turned on” by the introduction of the trans-activator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The zygote is the best target for micro-injection. In the mouse, the male pronucleus reaches the size of approximately 20 micrometers in diameter which allows reproducible injection of 1-2 p1 of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic non-human animal will carry the incorporated transgene. This will in general also be reflected in the efficient transmission of the transgene to offspring of the founder since 50% of the germ cells will harbor the transgene. Microinjection of zygotes is the preferred method for incorporating transgenes in practicing the invention.

Retroviral infection can also be used to introduce a WT1-BP transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally segregate in the offspring and are further inherited in a Mendelian fashion. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

Methods of making knock-out or disruption transgenic animals are also generally known. See, for example, Hogan, B., Manipulating the Mouse Embryo, (Cold Spring Harbor Laboratory Press, Cold Spring Harbor, N.Y., 1986). Recombinase dependent knockouts can also be generated, e.g. by homologous recombination to insert recombinase target sequences, such that tissue specific and/or temporal control of inactivation of a gene can be controlled as above.

The invention is further illustrated by the following examples, which should not be construed as further limiting. The contents of all references, pending patent applications and published patents, cited throughout this application, are hereby expressly incorporated by reference.

### EXEMPLIFICATION

Materials and Methods

The following methodology described in the Materials and Methods section was used throughout these Examples, set forth below.

Isolation of WT-1-interacting proteins

Escherichia coli (E. coli) and Saccharomyces cerevisiae were manipulated essentially as described (Ausubel, F. M. et al. (ed.) (1993) Current protocols in molecular biology., vol. 2. John Wiley & Sons, Inc). EGY48 MATa trp1, ura3, his3, LEU2::pLexAop6-LEU2 was used as a host for all interaction experiments (Zervos, A. S. et al. (1993) Cell 72:223-232). Yeast plasmids were rescued into E. coli K-12 strain KC8 pyrF::Tn5, hsdR, leuB600, trpC9830, lacD74, strA, galK, hisB436 as described (Zervos, A. S. et al. (1993) Cell 72:223-232). The full-length WT1 A cDNA was inserted into pEG202 at the EcoRI site and the generated plasmid pEG-WT1 was used as the bait. The oligo-primed HeLa cDNA yeast expression library was screened essentially as described (Zervos, A. S. et al. (1993) Cell 72:223-232).

Fresh yeast colonies from His⁻Ura⁻Trp⁻ plates (defined medium with glucose or galactose as energy source) were lifted onto nitrocellulose membranes and lysed by submersing in liquid nitrogen for 1 min. The membranes were then placed on top of Whatman filter papers saturated with 3 ml of Z-buffer (100 mM sodium phosphate, pH 7.0, 10 mM KCl, 1 mM MgSO4, 40 mM β-mercaptoethanol) containing 1 mg/ml 5-bromo-4-chloro-3-indolyl-β-D-galactopyranoside (X-gal). The membranes were incubated at room temperature and the color of colonies was recorded through the course of 30 min.

Cells and Transfection Methods

HeLa and 293 cells were grown in Dulbecco's modified Eagle medium (DMEM) supplemented with 10% heat inactivated calf serum (for HeLa cells ) or fetal calf serum (for 293 cells). The cells were transfected by the calcium phosphate precipitation method (Shi, Y. et al. (1991) Cell 67:377-388). The total amount of DNA was adjusted with the plasmid pSP72 to be identical for each transfection. Cells were harvested 48 hr after addition of the precipitate. All transfection assays were carried out with at least two independent DNA preparations and were repeated between three and five times. Human melanoma cells A375-C6 were cultured in RPMI 1640 medium supplemented with 10% serum and transfected as described (Shi, Y. et al. (1991) Cell 67:377-388). In cotransfection experiments equal amounts of each plasmid DNA was used. Transfectants were selected in culture medium supplemented with 300 μg/ml of G418 sulfate (Life Technologies, Inc., Gaithersburg, Md.). To quantify the effect of WT1 and Par-4 on growth of A375-C6 cells, the transfected cells were seeded at a density of 2,000 cells/200 μl in 96-well plates and grown for 72 h. Thereafter, the cells were pulsed with [³H]thymidine for 8 h and subjected to [³H]thymidine incorporation assays as described (Sells, S. F. et al. (1995) Mol. Cell. Biol. 15:682-692).

ChloramRhenical acetyl transferase (CAT) Assays

Whole cell extracts were prepared from transfected cells. CAT activity was assayed as described (Shi, Y. et al. (1991) Cell 67:377-388) and quantitated with a Beckman LS6500 scintillation counter. Proper amounts of cell extracts were used to measure CAT activity to ensure that the assays were performed within linear range.

Northern Blotting

Nitrocellulose filters containing approximately 2 μg of poly A⁺ RNA per lane from sixteen different adult human tissues (Clontech, Palo Alto, Calif.) were used for Northern analysis. Filters were prehybridized and hybridized in 50% deionized formamide, 5×Denhardts' solution, 5×SSPE, 0.5% SDS, 100 μg/ml denatured salmon sperm DNA at 42° C. Blots were hybridized with ³²P labeled human par-4, WT1 or β-actin cDNA probes for 16 hr at 42° C. The filters were washed twice in 2×SSC, 0.1% SDS for 30 min at room temperature and twice in 0.2×SSC, 0.1% SDS for 30 min at 65° C. for 30 min.

Immunoprecipitation/Western Blotting Assays

293 cells were cotransfected with 15 μg of pCMVFLAG-par-4 together with either 15 μg of pRSV-WT1 or 15 μg of the RSV vector DNA. After 48 hr, cells were lysed in a buffer containing 25 mm HEPES (pH7.0), 0.25 M NaCl, 2.5 mM EDTA, 0.5 mM DTT, 10 μg/ml leupeptin, 1 μg/ml pepstatin A, 2 mM phenylmethylsulfonyl flouride and 0.1% NP-40 for 30 min on ice. Extracts were incubated with WT1 antibodies (C19, Santa Cruz Biotechnology Inc., CA) or normal rabbit serum (NRS) overnight and immune complexes were collected with protein A-Sepharose beads at 4° C. for 1 hr. The beads were washed eight times with lysis buffer and the proteins eluted with Laemmli sample buffer. Proteins were separated by electrophoresis through a 10% polyacrylamide gel, transferred onto Immobilon PVDF membrane (Millipore, Bedford, Mass.) and probed with 0.1 μg/ml monoclonal anti-FLAG antibody (Kodak, New Haven, Conn.). Blots were incubated with alkaline phosphatase coupled goat anti-mouse antibody (BioRad, Hercules, Calif.) and immunoreactive proteins were visualized with 5-bromo4-chloro-3-indolylphosphate-nitro blue tetrazolium (Boehringer Mannheim, Mannheim, Germany).

For the analysis of the interaction between endogenous WT1 and par-4, M15 cells (80% confluency) were lysed in ELB buffer (25 mM HEPES [pH 7.0], 250 mM NaCl, 2.5 mM EDTA, 1 mM sodium orthovanadate, 50 mM B-glycerophosphate, 0.1% Nonidet P40 containing 2 mM phenylmethysulfonyl fluoride and 10 μg each of aprotinin and leupeptin per ml) for 30 min on ice. Lysates were clarified by centrifugation at 13,000 rpm for 15 minutes at 4 C. and incubated with a control monoclonal antibody (12CA5) or mouse anti-human Par-4 monoclonal antibody (A10, Santa Cruz Biotechnology, Inc.). After rocking lysates overnight, protein A-Sepharose beads were added and incubation continued for another 30 min at 4 C. Immunoprecipitates were washed four times in ELB buffer, denatured in SDS-gel loading buffer and fractionated on a SDS-12% polyacrylamide gel. Proteins were blotted onto nitrocellulose and the filters blocked with 5% nonfat milk in TBS-T (20 mM Tris-HCl [pH 7.5], 150 mM NaCl, 0.1% Tween 20) for 2 hours at room temperature. Primary antibody interaction was carried out by an overnight incubation of blots with a rabbit anti-WT-1 polyclonal antibody (C-19, Santa Cruz Biotechnology Inc.). After the binding of peroxidase-conjugated goat anti-rabbit immunoglobin G (Fab′)₂ secondary antibody (Jackson ImmunoResearch Laboratories, Inc.) for 1 hour in TBS containing 1% nonfat milk, filters were washed in TBS-T and developed with Supersignal CL-HRP Substrate System (Pierce) according to manufacture instructions.

GST-based assays of the WT1/par-4 interaction

All GST fusion proteins were purified as described (Kaelin Jr., W. G. et al. (1991) Cell 64:521-531) and the yield of each protein was determined by SDS-PAGE analysis and Coomassie blue staining. GST proteins bound to glutathione-agarose beads (Sigma, St. Louis, Mo.) were washed twice in NET-50 (20 mM Tris pH8,0, 1 mM EDTA, 50 mM NaCl) for 15 min at room temperature and were incubated in 200 μl binding buffer [25 mM HEPES pH7.5, 12.5 mM MgCl₂, 20% glycerol, 0.1%NP-40, 150 mM KCl, 1 mM dithiothreitol (DTT), 150 μg/ml bovine serum albumin (BSA), 200 μg per ml ethidium bromide (EtBr)] for 10 min at room temperature. In vitro transcription/translation reactions (Promega, Madison, Wis.) containing ³⁵S-methionine (1175 Ci/mmol) were programmed with pSP64-WT1 or pGem7zf(+)-YY1 and 5 μl was incubated with immobilized GST-fusion proteins for 1 hr at room temperature as indicated. The beads were washed five times with 1 ml washing buffer (20 mM Tris pH8.0, 100 mM NaCl, 1 mM EDTA, 0.5% NP-40, 200 μg/ml EtBr). Bound proteins were eluted with Laemmli sample buffer, separated on a 10% SDS-polyacrylamide gel which was soaked in the fluorographic solution “Amplify” purchased from Amersham and visualized by fluorography.

To map the region of WT1 that interacts with par-4, various GST-WT1 fusion proteins were produced as above, and 293 cells transfected with 10 μg pCMVFLAG-par-4 were lysed in lysis buffer (25 mM Hepes, pH7.0, 0.25 M NaCI, 2.5 mM EDTA, 0.5 mM DTT, 10 mg/ml leupeptin, 1 mg/ml pepstatin A, 2 mM phenylmethylsulfonyl fluoride, 0.1% NP40). Approximately 1 μg of each fusion protein was incubated with 200 μg of whole cell lysate. Beads were washed five times in 1 ml washing buffer as above, proteins were separated by SDS-PAGE and bound FLAG-par-4 was visualized by Western blot as above.

Detection of par-4 and WT1 in nuclear and cytoplasmic fractions

Contransfections of 293 cells with 10 μg each of pCMVFLAG-par-4 and pRSV-WT1 were performed, and cytoplasmic and nuclear fractions were prepared (Dignam, J. D. et al. (1983) Nucleic Acids Res 11:1475-1489). Lysates were quantitated for protein by Bradford assay and equal amounts of cytoplasmic and nuclear proteins were separated by SDS-PAGE. Subsequent Western analyses were performed with either an anti-FLAG monoclonal antibody (Kodak, New Haven, Conn.) or anti-WT1 antibody (Santa Cruz Biotechnology, Santa Cruz, Calif.).

Production of affinity-purified α-par-4 polyclonal antibodies

Rabbits were immunized with human GST-par-4 as described previously (Harlow, E. et al. (1988) Antibodies, A Laboratory manual Cold Spring Harbor Press). Following three boosts with the GST-PAR-4, rabbit sera was tested for reactivity with par-4 by immunoprecipitation and Western blot. Affinity purified α-par-4 antibodies were obtained by using immunoaffinity columns containing GST-par-4 and GST attached to cyanogen bromide-agarose beads (Harlow, E. et al. (1988) Antibodies, A Laboratory manual Cold Spring Harbor Press).

Interaction Trap

A general transcription-based selection for protein-protein interactions was used demonstrate self-association of WT1 and to isolate cDNA encoding proteins able to bind to WT1. Development of the “interaction trap assay” is described in, for example, Gyuris et al. (1993) Cell 75:791-803; Chien et al. (1991) PNAS 88:9578-9582; Dalton et al. (1992) Cell 68:597-612; Durfee et al. (1993) Genes Dev 7:555-569; Vojteck et al. (1993) Cell 74:205-214; Fields et al. (1989) Nature 340:245-246; and U.S. Pat. No. 5,283,173). As carried out in the present invention, the interaction trap comprises three different components: a fusion protein that contains all or a portion of WT1 and the LexA DNA-binding domain (the “bait”); reporter genes that have no basal transcription and whose transcriptional regulatory sequences are dependent on binding of LexA; and a second fusion protein comprising a WT1 polypeptide or proteins encoded by a HeLa cDNA library, the fusion protein having an amino terminus containing an activation domain and other useful moieties (the “fish”). Briefly, baits were produced constitutively from a 2μ HIS3+ plasmid under the control of the ADH1 promoter and contained the LexA carboxy-terminal oligomerization region, which contributes to operator occupancy by LexA derivatives. Baits were made in pLexA(1-202)+PL (described in Ruden et al. Nature (1991) 350:250-252; and Gyuris et al. Cell (1993) 75:791-803) after PCR amplification of the bait coding sequences from WT1 (see Table I). PCR primers provided WT1 fragments with restriction markers for cloning into pLexA(1-202)+PL as EcoR1-BamH1, EcoR1-Sal1, EcoR1-Xho1 or BamH1-Sal1 fragments. When EcoR1 is used, there are two amino acids inserted (EF) between the last amino acid of LexA and the bait moieties. BamH1 fusion results in five amino acid insertion (EFPGI) between LexA and the fused protein.

Reporters

The LexAop-LEU2 construction replaced the yeast chromosomal LEU2 gene. The other reporter, pRB1840, one of a series of LexAop-GAL1-lacZ genes (Brent et al. (1985) Cell 43:729-736; Kamens et al. (1990) Mol Cell Biol 10:2840-2847), was carried on a 2μ plasmid. Basal reporter transcription was extremely low, presumably owing both to the removal of the entire upstream activating sequence from both reporters and to the fact that LexA operators introduced into yeast promoters decrease their transcription (Brent and Ptashne (1984) Nature 312:612-615). Reporters were chosen to differ in sensitivity. The LEU2 reporter contained three copies of the high affinity LexA-binding site found upstream of E. coli Co1E1, which presumably bind a total of six dimers of the bait. In contrast, the lacZ gene contained a single lower affinity operator that binds a single dimer of the bait. The operators in the LEU2 reporter were closer to the transcription start point than they were in the lacZ reporter. These differences in the number, affinity, and position of the operator contribute to increased sensitivity of the LEU2 reporter compared to the lacZ gene.

Expression Vectors and Library

Library proteins were expressed from pJG4-5, a member of a series of expression plasmids designed to be used in the interaction trap and to facilitate analysis of isolated proteins. These plasmids carry the 2μ replicator and the TRP1 marker. pJG4-5, shown in FIG. 1, directs the synthesis of fusion proteins. Proteins expressed from this vector possess the following features: galactose-inducible expression so that synthesis is conditional; an epitope tag to facilitate detection; a nuclear localization signal to maximize intranuclear concentration to increase selection sensitivity; and an activation domain derived from E. coli (Ma and Ptashne (1987) Cell 57:113-119). The activation domain was chosen because its expression and activity are not subject to known regulation by yeast proteins, and because it is weak enough to avoid toxicity (Gill and Ptashne (1988) Nature 334:721-724; Berger et al. (1992) Cell 70:251-265) that might restrict the number or type of interacting proteins that are recovered.

EcoRI-Xhol cDNA-containing fragments, generated from total HeLa cell cDNA, were introduced into the pJG4-5 plasmid. Alternatively, the coding sequence for WT1 was ligated into pJG4-5, in frame with the B42 transcriptional activation domain.

### EXAMPLE 1

WT1 self-associates via a domain overlapping with its repressor domain.

WT-1-mediated repression of the PDGF-A chain and the human p18 promoters requires binding of WT1 to both the 5′ and 3′ sites flanking the transcription start sites of the promoters. This finding raised the possibility to us that self association of WT1 may be important for its repressor function. We wished to determine whether WT1 self associates and whether self-association may underlie its repression function. The yeast “two hybrid” system was used to explore WT1/WT1 interactions. WT1 utilizing cDNA was cloned into both the “bait” and the “prey” plasmids. In this embodiment, when each of the WT1 domains interacts with the other, the acidic activation domain should be brought to the promoter, leading to activation of the lacZ gene.

As shown in Table 1, the LexA-WT1 protein encoded by the bait plasmid (pEG202/WT1) alone did not activate the lacZ gene. However, WT1 protein was supplied when both on the bait and the prey (pJG4-5/WT1) plasmids, lacZ gene expression was induced, as determined by color assays (Zervos et al. (1993) Cell 72:223-232). This shows that WT1 protein-protein interactions are necessary for function. Neither controls pJG4-5/WT1 nor the acidic activator alone (pJG4-5), fused to the LexA DNA-binding domain, activated the lacZ gene (Table 1).

To map the domain of interaction, amino acid residues 1-180 of WT1 (containing the repressor domain), residues 175-301 (activation domain) and residues 275-429 (zinc finger domain) were each cloned into the bait plasmid pEG202. As shown in Table 1, pJG4-5/WT1 interacted with the fill length WT1 as well as WT 1-180, but not with the LexA protein alone nor the rest of the WT1 protein including the activation domain (residues 175-301) and the DNA-binding domain (residues 275-429). These results show that residues 1-180 of WT1 are involved in WT1 self association. The domain important for self association seems to overlap with the repressor domain previously defined by transfection assays (Madden et al. (1993) Oncogene 8:1713-1720; and Wang et al. (1993) J. Biol. Chem. 268:9972-9975). The same results were obtained also from a GST affinity matrix-based assay.

TABLE 1
WT1 self-association in yeast
Lac Z activity

| | pJG4-5 | pJG4-5/WT1 |
|---|---|---|
| Lex A alone | −− | −− |
| Lex A-WT1 | −− | ++++ |
| Lex A-WT1/1-180 | −− | ++++ |
| Lex A-WT1/175-301 | −− | −− |
| Lex A-WT1/275-429 | −− | −− |

Bait and prey plasmids were transformed in pairs into the yeast strain EGY48 containing the reporter LexAop-LacZ. Blue color of the colonies is indicated by −− or ++++ signs.

### EXAMPLE 2

Identification of WT1-interacting proteins.

We also used the “two hybrid” system to identify other cellular WT1-binding proteins. The LexA-WT1 construct, described above, was used as a “bait”, and the pJG4-5 cDNA library was used as a source for interacting proteins. Approximately 2×10⁶ independent colonies were screened, and a number of candidate proteins were identified based on their apparent ability to interact with WT1, but not with another zinc finger repressor/activator YY1 (Shi et al. (1991) Cell 67:377-388) or the p85 subunit of the P13 kinase (Skolnick et al. (1991) Cell 65:83-90) in yeast (Table 2). The specificity of the interactions was confirmed by independent protein interaction assays.

The cDNA plasmids that were identified as encoding proteins that specifically interacted with WT1 were subjected to DNA sequence analysis using a primer initiating within the B42 coding sequence. This provides information on the reading frame at the fusion point with the cDNA library insert. In general, this primer was used to obtain about 200-300 nucleotides of cDNA sequence. In each case an open reading frame for the insert was identified. Sequencing with primers appropriate for determination of the 3′ end of the cDNA insert was also performed.

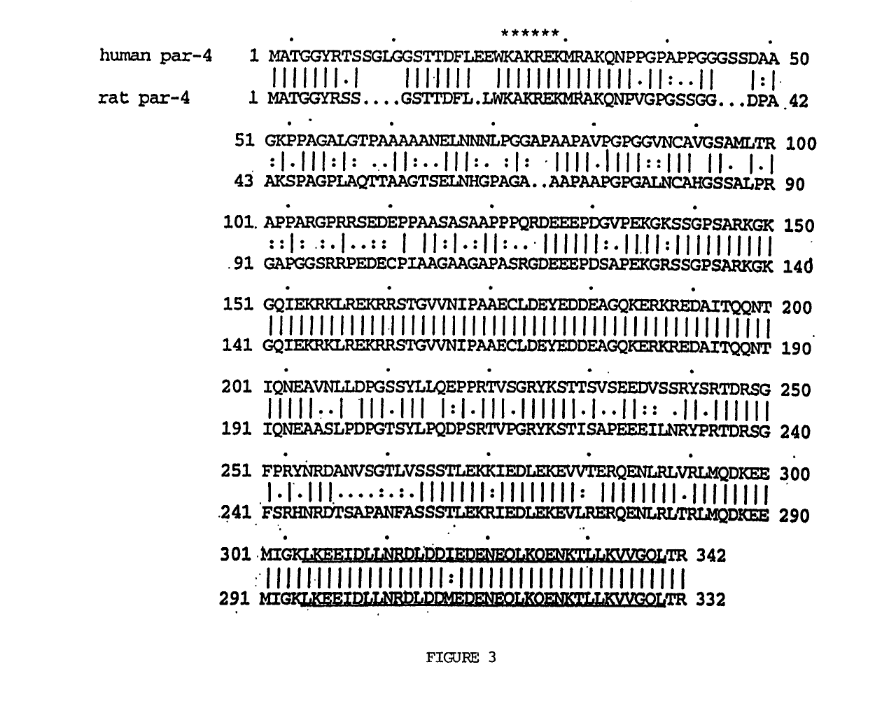

Sequence analysis indicated that an isolate termed “H2-73” encodes a polypeptide homologous to the rat par-4 protein (prostate apoptotic response) believed to be involved in apoptosis (Sells et al. (1994) Cell Growth Diff5:457466). A putative WT1-interacting protein encoded by the insert in isolate H2-73 was found to be a polypeptide that is 96% identical to the C-terminus (residues 280-332) of a rat protein termed par-4 (Sells, S. F. et al. (1994) Cell Growth & Dif. 5:457-466). The interaction of this partial par-4 protein with WT1 is specific, as it did not interact with another zinc finger repressor/activator YY1 (Shi, Y. et al. (1991) Cell 67:377-388) or the p85 subunit of P13 kinase (Skolnik, F. Y. et al. (1991) Cell 65:83-90). Using the isolated H2-73 cDNA as a probe, a full length human par-4 cDNA was isolated and its amino acid sequence was compared with that of the rat par-4 (FIG. 3). The human and the rat par-4 share 85% similarity throughout the entire coding region.

The nucleotide sequence of full length human par-4 cDNA (SEQ ID No.: 2) was deposited in Genbank under the accession number U63809. The rat par-4 sequence can be obtained under accession number U05989.

An isolate H3-45 (Ciao-1) contains an insert encoding a polypeptide that includes previously characterized repeat motifs, and is described in Example 4 below.

TABLE 2
Interactions Between WT1 and two binding protein clones in yeast
Lac Z Activity

| Interactors | p85 | YYI | WT1 |
|---|---|---|---|
| H2-73 (par-4) | −− | −− | +++ |
| H3-45 (Ciao-1) | +/− | −− | +++ |

### EXAMPLE 3

Genetic manipulations and sequence determination of cloned WT1-Binding Proteins.

The plasmids used for fusion to the bait (p85, YY1, WT1) and for the newly cloned prey (potential interactors) plasmids were transformed in pairs into the yeast strain EGY48 containing the reporter LexAop-LacZ. Results for the two clones described above are shown in Table 2, in which the extent of the blue color of the colonies is indicated by −−, ± or +++ signs. The data in the table show that the proteins encoded by each of the par-4 and the Ciao-1 isolates interact in vivo with the WT1 protein, and not with control proteins p85 and YY 1.

The cDNAs of the clones selected from the library were inserted into this vector as EcoR1-Xho1 fragments. The EcoR1 adaptor sequence is 5′-GAATTCTGCGGCCGC-3′ (SEQ ID No.:5) and the open reading frame encoding the interacting protein starts with the first G. With this information in hand, one of ordinary skill in the art can generate the subject recombinant WT1-BP genes and express recombinant forms of the subject WT1-binding proteins. Further, the WT1-binding proteins of the present invention can be amplified by PCR using the following primers:

5′-TAC CAG CCT CTT GCT GAG TGG AGA-3′ (SEQ ID No.:6)
5′-TAG ACA AGC CGA CAA CCT TGA TTG-3′ (SEQ ID No.:7)

It is evident to one of ordinary skill in the art, given the guide to the 5′ and 3′ ends to each of the clones provided in Table 3, that these clones and homologs and orthologs and polymorphisms of each, can be isolated using primers based on the nucleotide sequences provided by SEQ ID Nos.: 1 and 2, and SEQ ID Nos.: 6 and 7, using suitable combinations of appropriate primers. Clones of these can of a subject human or animal can be obtained from cDNA made from RNA from appropriate tissues, or from genomic DNA from cells, by PCR amplification or hybridization procedures described herein.

Isolated clones can be subcloned into expression vectors in order to produce a recombinant protein, or can be used to generate anti-sense constructs, or can be used to generate oligonucleotide probes. In an illustrative embodiment, oligonucleotide probes have been generated using the coding sequences for each of the clones of the subject invention, and used in Southern hybridization and in situ hybridization assays to detect the pattern and abundance of expression of each of the WT1-binding proteins.

Moreover, because each clone carries a plasmid encoding a fusion protein identified from an interaction trap assay, the clone can be utilized directly, for example, in a drug screening assay, or alternatively, in a mutagenesis procedure for mapping WT1 binding epitopes.

These sequences are also used to in combination with sequences of related proteins for which three-dimensional structure information has been obtained, for example by NMR, or X-ray crystallography. A comparison of the sequences and superposition of the structure of the known protein are useful for further engineering of WT1-binding protein determinants, for example, that have greater affinity to WT1 or other transcriptional effectors, using recombinant methods. The structural information thus obtained can be used also in modeling studies, to develop mimetic drugs using rational drug design, and for additional applications described in the instant invention. See for example these and other methods described in U.S. Pat. Nos. 5,223,409 and 5,096,815 by Ladner et al.

TABLE 3
Guide to pSHI- WT1BP

| Clone | Nucleotide Sequence | Peptide Sequence | Name |
|---|---|---|---|
| Ciao-1 | SEQ ID No. 1 | SEQ ID No. 3 | WT1-BP^{Ciao-1} |
| par-4 | SEQ ID No. 2 | SEQ ID No. 4 | WT1-BP^{par-4} |

### EXAMPLE 4

Ciao-1 is a member of the WD-40 family of proteins.

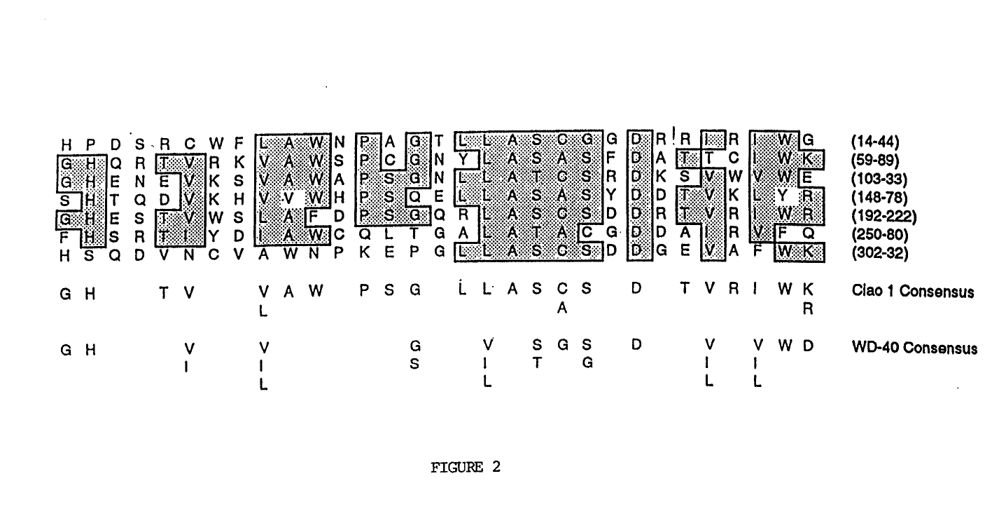

Ciao-1 encodes a protein with theoretical molecular weight of 37.7 KD (see SEQ ID No.: 3). The deduced protein sequence of Ciao-1 contains seven WD-40 repeats and their alignment with the WD-40 consensus is shown in FIG. 2. Evidence that the Ciao-1 cDNA clone comprises the complete coding region for Ciao-1 is based on the following findings: 1) Northern analysis indicated that Ciao-1 mRNA is 1.7 kb, while the cDNA clone obtained is 1.4 kb. Since the poly (A) stretches are usually 100-200 bases long, the 1.4 kb cDNA is therefore near full-length; 2) a translation initiation sequence is located near the 5′ end of the cDNA that conforms to the Kozak consensus (Kozak et al. (1984) Nature 308:241-246). In vitro translation experiments suggested that this AUG can be efficiently used, and results in a protein of the same molecular weight as Ciao-1 detected in vivo (described below). Taken together, the isolated cDNA clone most likely contains the complete coding information for Ciao-1.

### EXAMPLE 5

Physical interaction between Ciao-1 and WT1.

As described above, a clone of Ciao-1, H3-45, was shown in yeast “two hybrid” assays to interact specifically with WT1 but not with another zinc finger-containing transcription repressor/activator, protein YY1 (see Table 2). The glutathione-S-transferase transferase (GST) affinity matrix-based assays (Kaelin et al. (1991) Cell 64:521-581) were used in an independent approach to assess the specificity of the interactions. Both WT1 and Ciao-1 were fused to GST and the fusion proteins were purified using the glutathione Sepharose resins. ³⁵S-labeled WT1 was incubated with GST, GST-Ciao-1, GST-p53, as well as with GST-RACK1, another WD-40 family member which binds activated protein kinase C (Ron et al. (1994) PNAS 91:839-843).

It was observed that radiolabeled WT1 bound each of proteins GST-Ciao-1 and GST-p53. Furthermore, WT1 failed to bind control GST, or the fusion protein GST-RACK 1. The RACK1 protein is composed of seven WD-40 repeat motifs, a structure very similar to that proposed for Ciao-1, thus serving as a stringent control for the specificity of the WT1/Ciao-1 interaction. GST-Ciao 1 was also assayed for its ability to bind YY1, and binding was not observed. Taken together, these data show that the interaction observed here between WT1 and Ciao-1 is specific.

### EXAMPLE 6

Ciao-1 is predominantly a nuclear protein.

Most of the proteins involved in transcription are localized in the nucleus with a few exceptions, such as NFκB which is translocated from the cytoplasm into the nucleus under certain conditions. We determined the cellular localization of Ciao-1 by both indirect immunofluorescence staining and Western blotting. Full-length Ciao-1 cDNA was fused to the FLAG epitope tag (IBI), and then cloned into a cytomegalovirus (CMV) expression vector (Shi et al. (1991) Cell 67:377-388). Upon transfection of this plasmid into HeLa cells, the expression of the FLAG/Ciao-1 fusion protein was detected using monoclonal antibodies specific for the FLAG tag (α-FLAG M2, IBI).

Immunofluorescence demonstrated that the fusion protein was predominantly localized in the nucleus. Cells transfected with the CMV vector showed no detectable signals. Furthermore, fractionation of total cellular proteins into nuclear and cytoplasmic portions, followed by Western blotting analysis, also suggested nuclear localization of the Ciao-1 protein. This finding is consistent with a role for Ciao-1 in transcription.

### EXAMPLE 7

Initial characterization of tissues in which Ciao-1 is expressed in vivo.

We examined expression patterns of Ciao-1 by Northern analysis. It has been shown previously that, in addition to fetal kidney, WT1 is expressed in testis, and at lower level in spleen and adult kidney (Buckler et al. (1991) Mol. Cell Biol. 11:1701-1712; and Seto et al. (1991) Nature 345-:241-245). Total RNAs were isolated from these tissues (mouse) and from a human Wilms' tumor sample. These RNA samples, and RNA isolated from HeLa cells, 293 embryonic kidney cells, and K562 erythroleukemia cells, were probed with ³²P-labeled Ciao-1 cDNA fragments.

Ciao-1 mRNA was detected in kidney, testis, the Wilms' tumor sample and HeLa cells. Expression of Ciao-1 was also detected in 293 embryonic kidney cells and K562 erythroleukemia cells, cell lines where WT1 was expressed. Its expression in the spleen and the liver was lower than the level of detectability in the same assay. Therefore, Ciao-1 is indeed expressed in WT1-expressing tissues under physiological conditions. The negative result with liver and spleen, however, do not necessarily exclude them from being Ciao 1-expressing tissues. These observations are consistent with a physiological role for Ciao-1/WT1 interactions.

### EXAMPLE 8

Polyclonal antibodies against Ciao-1.

To facilitate further studies of Ciao-1/WT1 interactions, polyclonal antibodies against Ciao-1 were prepared. Full-length Ciao-1 was subcloned into GEX-2TK vector to create GST-Ciao-1 fusion proteins, which were purified by affinity chromatography using glutathione Sepharose resins. GST-Ciao-1 was injected into rabbits, rabbits were bled periodically to determine antibody titer, blood samples for antibody preparation were removed, and sera were prepared following standard procedures (Harlow, E. et al. (1988) Antibodies, A Laboratory manual, Cold Spring Harbor Laboratory Press).

The resulting serum from immunized rabbits, but not the preimmune serum, reacted with the in vitro translated Ciao-1, indicating that antibodies specific for Ciao-1 had been elicited. The serum was then used to probe HeLa nuclear extracts. The anti-Ciao-1 polyclonal serum, but not preimmune serum, recognized a 43 kD protein in HeLa cells. This recognized protein has the same MW (43 kD) as Ciao-1 translated in vitro. As a control, CMV-LAG/Ciao-1 and the CMV vector were transfected into HeLa cells and the lysates were probed with α-FLAG antibodies. The α-FLAG antibodies recognized the FLAG/Ciao 1 fusion protein in CMV-FLAG/Ciao 1 transfected but not the CMV vector transfected cell lysate. Again, the FLAG/Ciao-1 showed a similar electrophoretic mobility as the protein recognized by α-Ciao-1 antibodies. The reasons for the discrepancy between the deduced MW of Ciao-1 (37.7 kD) and that of Ciao-1 synthesized both in vivo and in vitro (43 kD) are unknown. Ciao-1 can have a structural feature that causes its aberrant migration on an SDS protein gel, a phenomenon observed with other proteins, for instance, YY1 which migrates as a 68 kD protein on SDS-PAGE gel electorphoresis even though it has a deduced MW of 44 kD (Shi et al. supra).

### EXAMPLE 9

Over-expression of HA-tagged WT1 in insect Sf9 cells.

To facilitate studies of WT1 interactions with other proteins, we constructed a recombinant baculovirus that expresses influenza hemaglutinin (HA) epitope tagged WT1. Full-length WT1 cDNA was first fused to a DNA fragment encoding the HA polypeptide to generate HA-WT1 which was then cloned into baculovirus vector pVL1392 (Invitrogen, CA). The recombinant viruses were isolated and used to infect insect Sf9 cells to determine the expression of HA-WT1.

Anti-WT1 antibodies were used to recognize a protein produced in insect cells with a molecular weight expected for HA-WT1, while the same antibody did not react with protein in control cells infected with either a recombinant virus carrying HA-YY1 , or control cells infected with the wild-type baculovirus. A duplicate blot was probed with α-HA antibodies 12CA5 (Field et al (1988) Mol. Cell. Biol. 8:2159-2165) which recognized both HA-WT1 and HA-YY1. Again, wild-type baculovirus infected cell extracts were used as a negative control. The HA-WT1 expressed in insect cells can be purified by affinity column chromatography using α-HA antibody coupled protein A-Sepharose resins, and provides a source of WT1 for the diagnostic assays and drug screens of the present invention.

### EXAMPLE 10

In Vitro and In Vivo Interactions between Par-4 and WT.

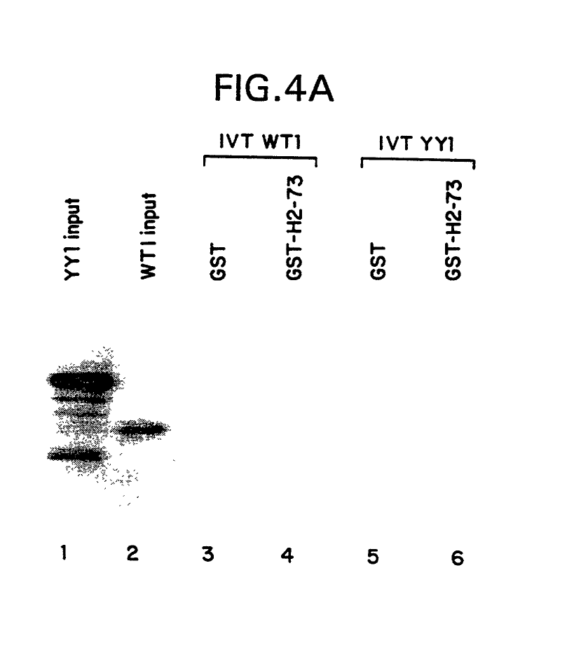

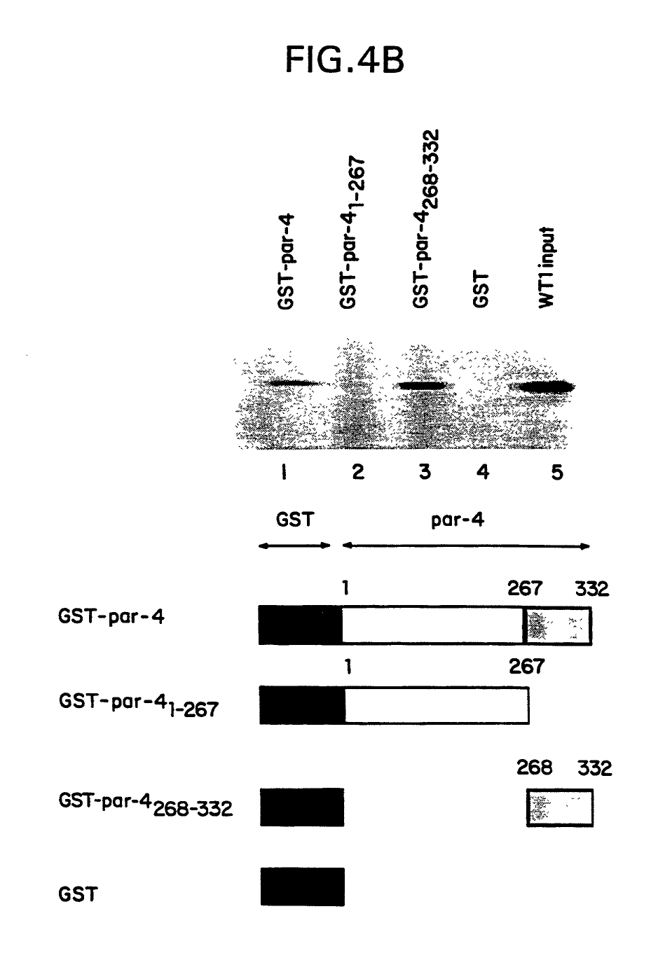

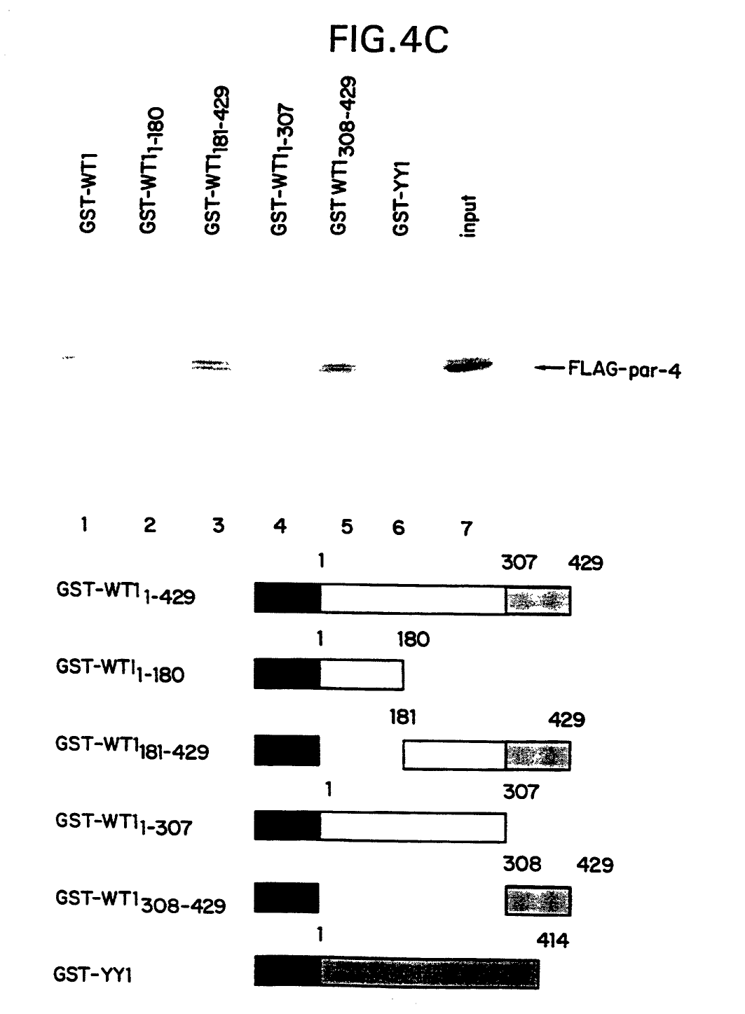

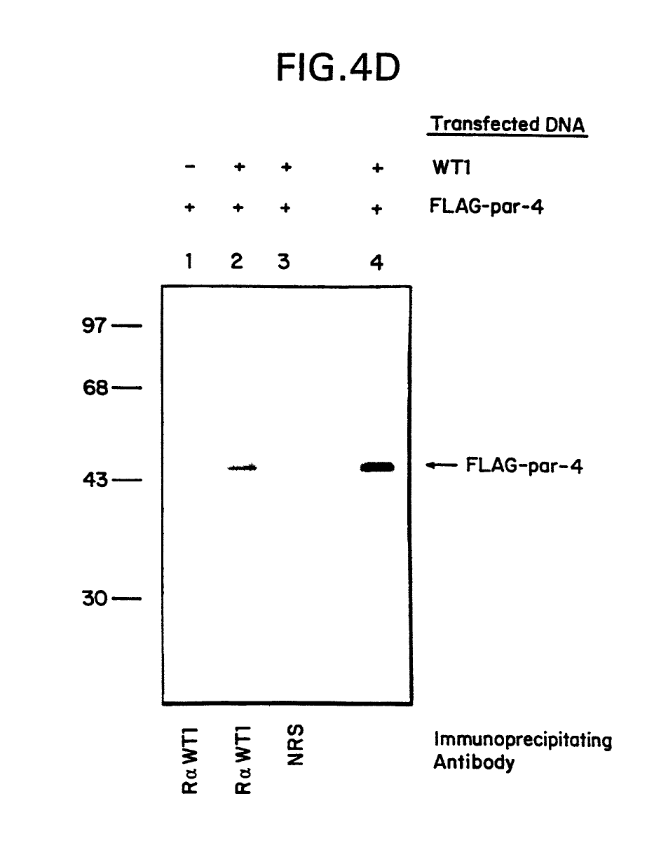

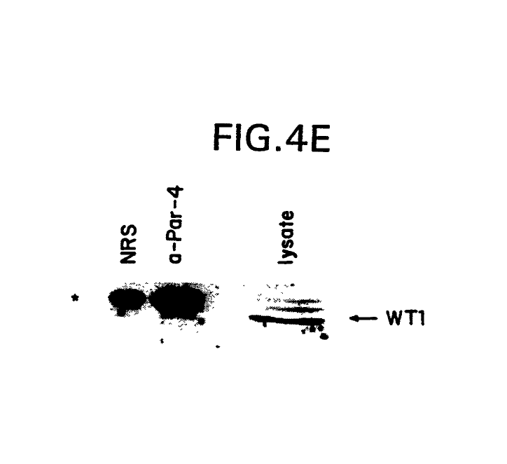

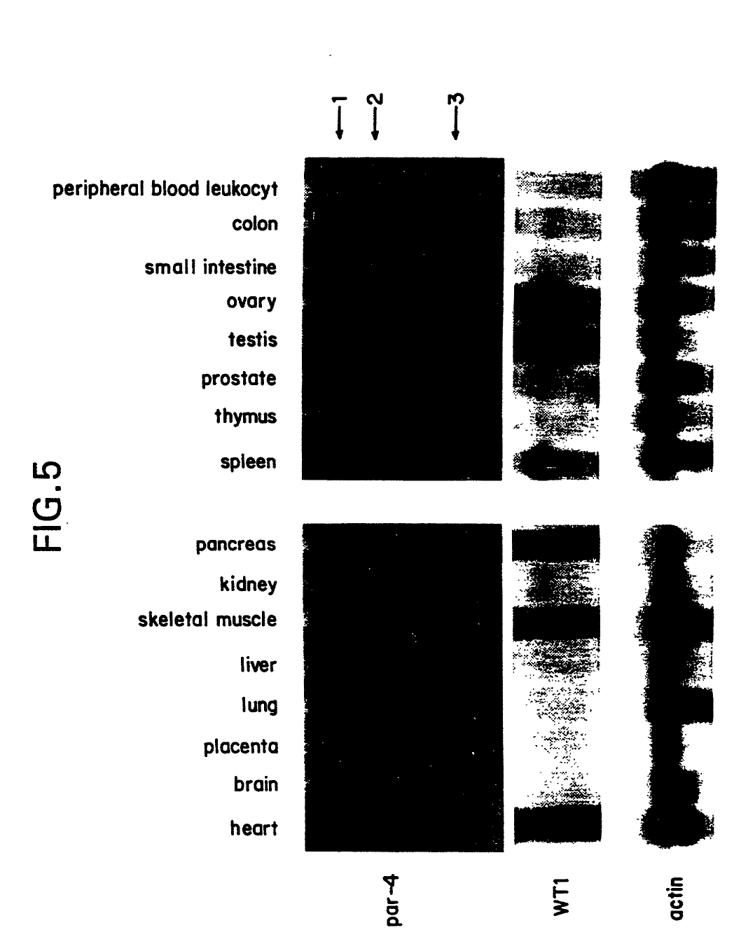

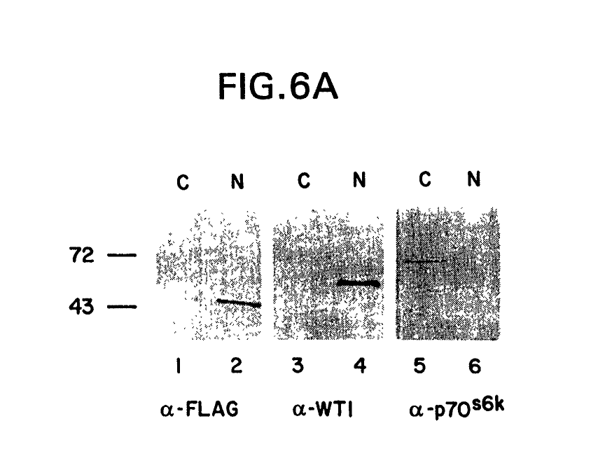

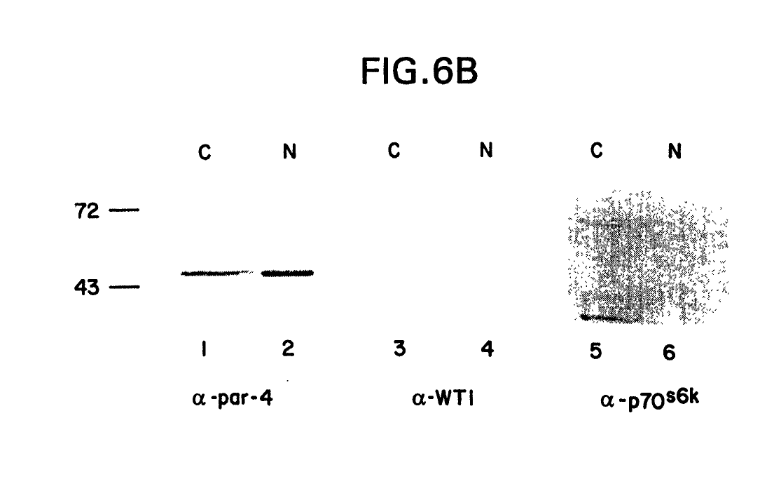

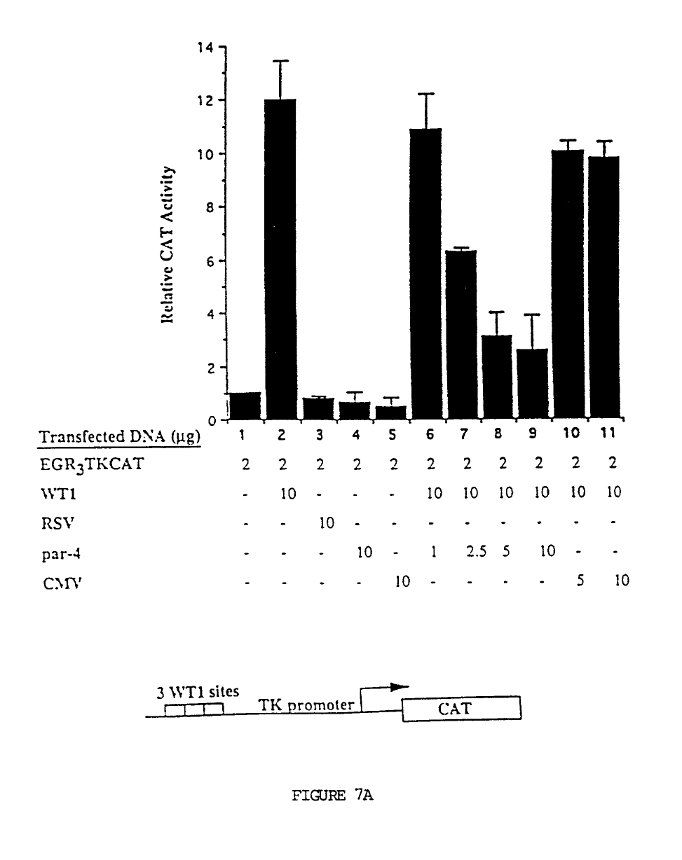

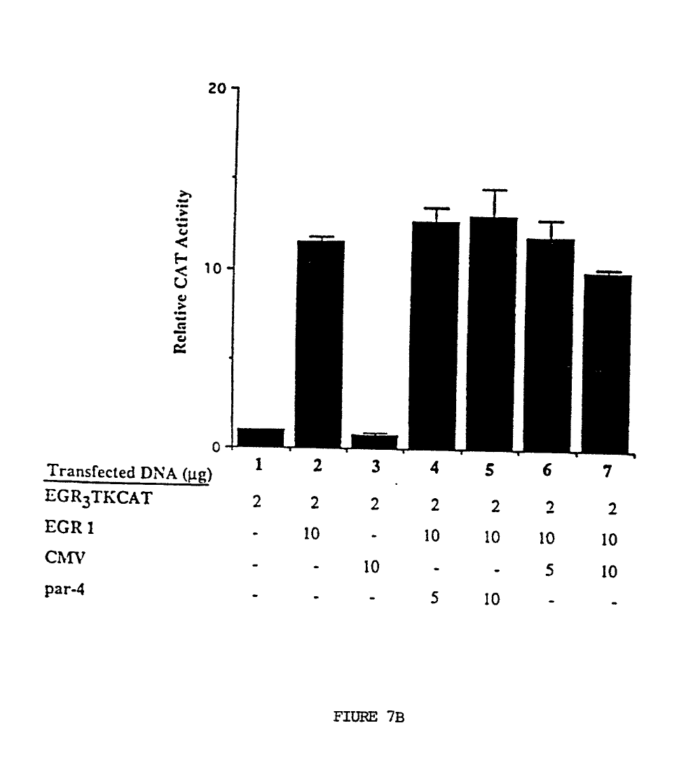

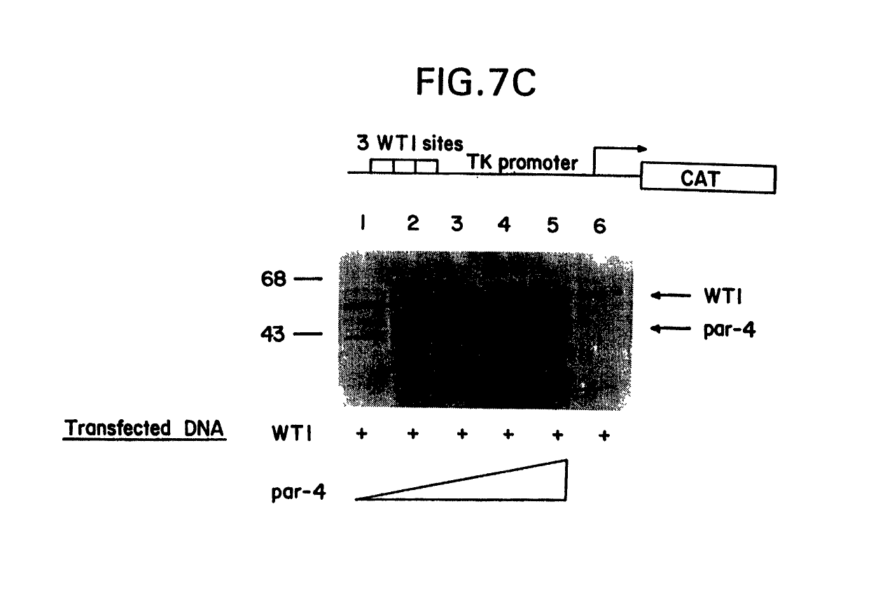

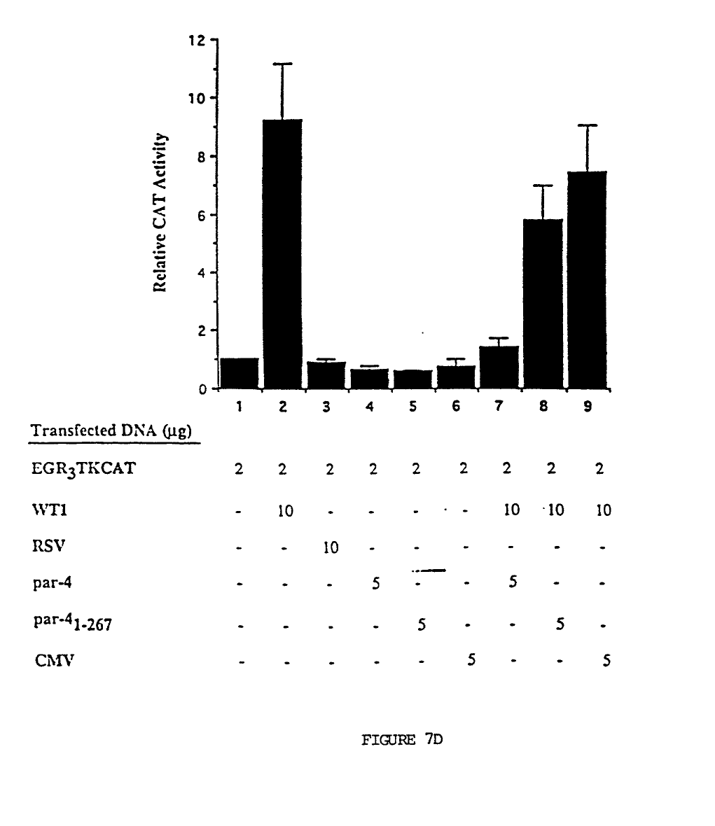

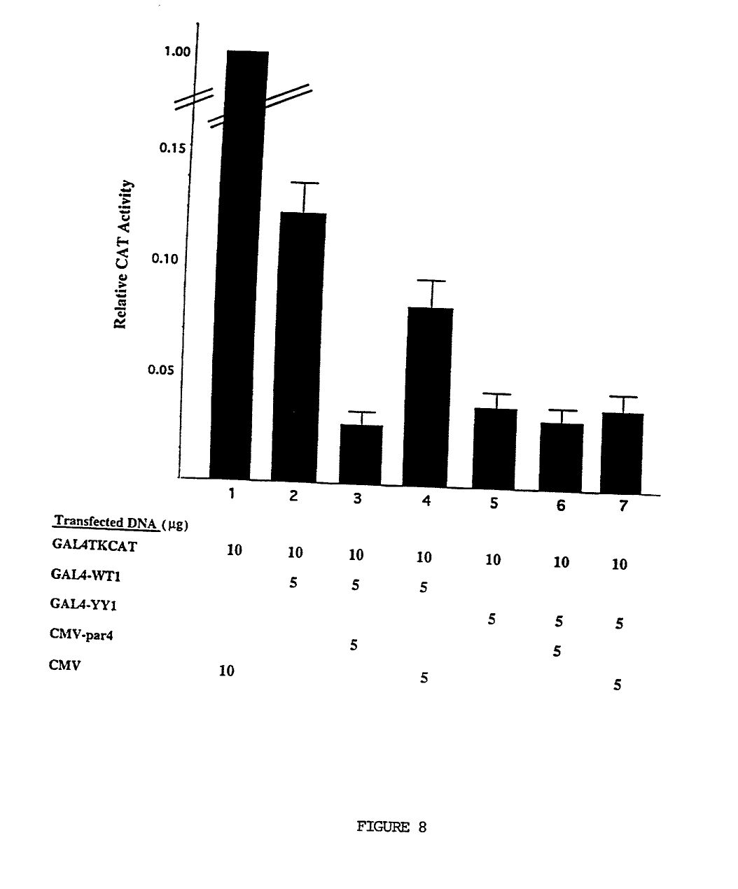

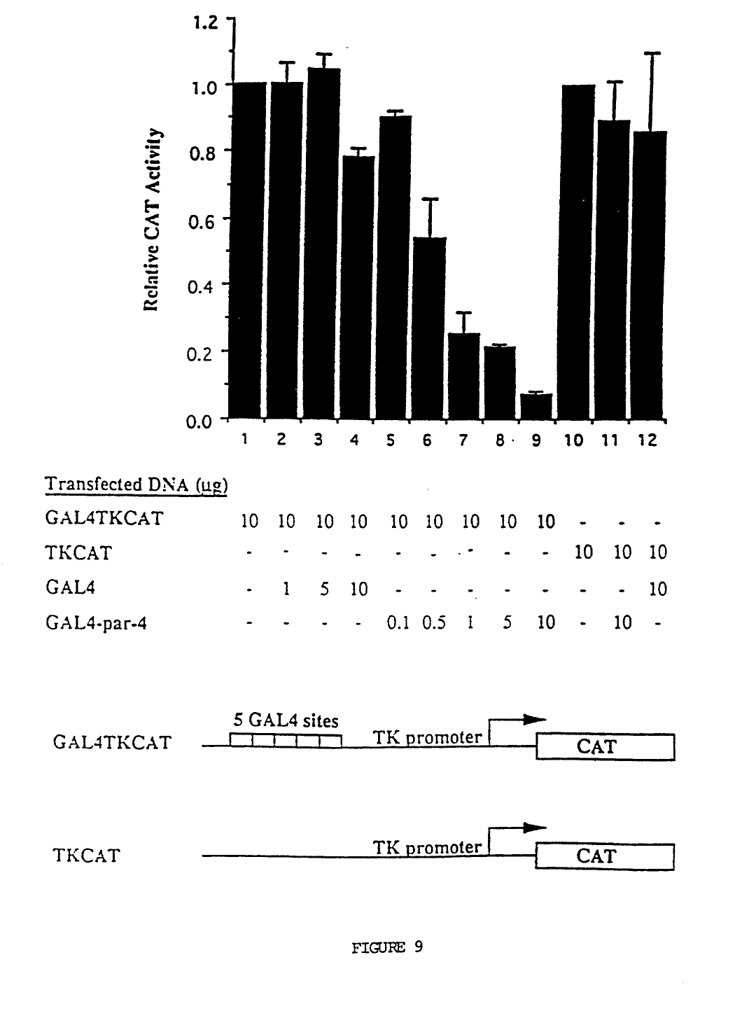

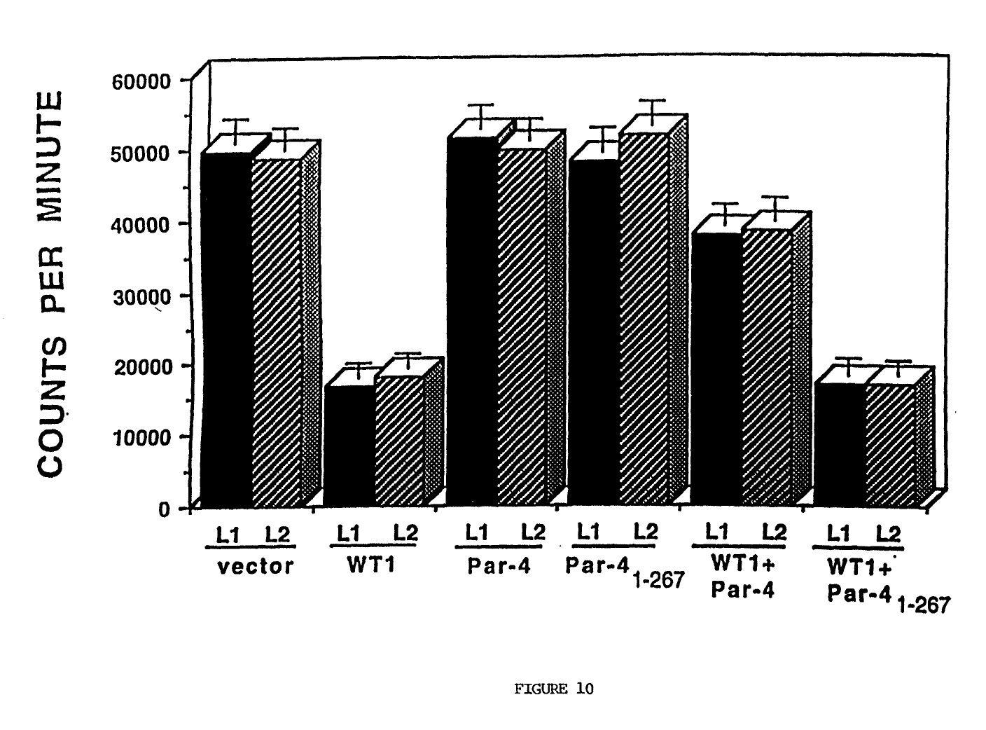

To confirm the WT1/H2-73 (par-4) interaction observed in yeast cells, GST fusion protein-based assays were carried out. As shown in FIG. 4A, in vitro translated WT1 (A) was specifically retained by GST-H2-73, but not by GST alone (lanes 3 and 4). As a specificity control, the zinc finger repressor/activator YY1 (Flanagan, J. R. et al. (1992) Mol. Cell. Biol. 12:38-44; Hariharan, N. et al. (1991) Proc. Natl. Acad Sci. USA 88:9799-9803; Park, K. et al. (1991) Proc. Natl. Acad. Sci. USA 88:9804-9808; Shi, Y. et al. (1991) Cell 67:377-388) failed to interact with par-4 under the same assay condition (FIG. 4A, lanes 5 and 6 ). To determine whether WT1 interacted with full length rat par-4 and to identify domains within WT1 and par-4 that mediate the physical interaction, further in vitro binding experiments were performed. The full length rat par-4, the N-(residues 1-267) and C-terminal (residues 268-332) domains of the rat par-4 were fused to GST. In vitro translated WT1 bound both GST-par-4 and GST-par-4₂₆₈₋₃₃₂ (FIG. 4B, lanes 1 and 3) but failed to interact with GST-par-4₁₋₂₆₇ (FIG. 4B, lane 2). Therefore, residues 268-332 of par-4 are necessary and sufficient to mediate the physical interaction between par-4 and WT1. In addition, WT1 isoforms B, C and D were found to bind par-4 in a similar manner as the WT1 A isoform (data not shown).

To map the region of WT1 that is involved in the physical interaction with par-4, par-4 was expressed in 293 cells, a human embryonic kidney cell line (Graham, F. et al. (1977) J. Gen. Virol. 36:59-72), as a fusion protein with a FLAG epitope attached to the amino terminus (FLAG-par-4). Whole cell lysates were incubated with purified GST-WT1, GST-WT1₁₋₈₀, GST-WT1₁₈₁₋₄₂₉, GST-WT1_{1-307,} GST-WT1₃₀₈₋₄₂₉ and GST-YY1. Bound FLAG-par-4 was detected by Western blotting using a monoclonal a-FLAG antibody (Kodak, New Haven, Conn.). As shown in FIG. 4C, FLAG-par-4 was captured specifically by the full length WT1, WT1₁₈₁₋₄₂₉ and WT1₃₀₈₋₄₂₉ (lanes 1, 3 and 5) but not by WT1₁₋₁₈₀ or WT1₁₋₃₀₇ (lanes 2 and 4). These results suggest that residues 308 to 429 of WT1 are necessary and sufficient to interact with par-4. This region of WT1 is composed of four zinc fingers that constitute the DNA-binding domain of WT1 (Call, K. M. et al. (1990) Cell 60:509-520; Gessler, M. et al. (1990) Nature 343:774-778; Rauscher III, F. et al. (1990) Science 250:1259-1263). Since FLAG-par-4 did not bind GST-YY1 which also contains zinc fingers at its C-terminus (FIG. 4C, lane 6), these findings indicate that par-4 binds specifically to the zinc finger domain of WT1, but not to zinc finger structure in general. Finally, two hybrid analysis also identified the zinc finger domain of WT1 as the par-4-interacting domain in yeast, consistent with the results obtained from the GST fusion protein-based assays described above.

The WT1/par-4 interaction was further examined in mammalian cells by co-immunoprecipitation assays. FLAG-par-4 was co-transfected with WT1 or RSV vector into 293 cells. Cells were lysed and WT1 was immunoprecipitated with α-WT1 polyclonal antibodies followed by Western blotting using α-FLAG antibodies to detect the presence of FLAG-par-4. As shown in FIG. 4D, FLAG-par-4 was specifically co-immunoprecipitated by α-WT1 antibodies (lane 2) but not by preimmune serum (lane 3).

Co-immunoprecipitation of par-4 was not due to antibody cross reactivity as par-4 was absent among proteins that were immunoprecipitated by α-WT1 antibodies from 293 cells that lacked transfected WT1 (FIG. 4D, lane 1).

We further examined the endogenous WT1/par-4 interaction in the M15 mouse mesonephric cell line M15 without overexpression of either proteins. Both WT1 and par-4 proteins are expressed in M15 cells (Larsson, S. H. et al. (1995) Cell 81:391-401), and FIG. 6B). Par-4 was immunoprecipitated from M15 whole cell lysates and the immunoprecipitate was analyzed for the presence of WT1 by Western blotting using a-WT1 antibodies. As shown in FIG. 4E, WT1 is coprecipitated using an α-par-4 monoclonal antibody (lane 2) but not normal rabbit serum (lane 1). This result, combined with the WT1/par-4 interaction in 293 cells, yeast two-hybrid and GST-binding data, demonstrates that WT1 and par-4 interact with one another both in vivo and in vitro.

Using the yeast two-hybrid approach, a clone designated as H2-73 was obtained that encoded a polypeptide capable of specifically interacting with WT1. A search of the database revealed 96% identity between H2-73 and the rat par-4 protein indicating that H2-73 was a partial clone of the human homologue of rat par-4. Subsequent GST-based assays confirmed the WT1/par-4 interaction observed in yeast cells (FIG. 4A-C). This interaction can also be recapitulated when par-4 and WT1 were co-expressed in 293 cells, a human embryonic kidney cell line. Significantly, the interaction between the endogenous WT1 and par-4 without overexpression can also be detected in mouse mesonephric cells (FIG. 4E). Judging from the amount of coprecipitated proteins, only a small percentage of either protein is found in the complex.

Further GST-binding analyses identified the domains of WT1 and par-4 that were involved in their physical interactions. The interaction of WT1 was mediated by the four C₂H₂-type zinc fingers located at its C-terminus (FIG. 4C). This interaction is clearly not due to the general zinc finger structure but is specific to the zinc fingers of WT1 as YY1, another C₂H₂-type zinc finger protein did not interact with par-4. The idea that zinc fingers, in addition to binding DNA/RNA, may also mediate protein/protein interaction has been demonstrated in other experimental systems. For instance, the zinc fingers of YY1 have been shown to be involved in physical interactions with Sp1 (Lee, J. -S. et al. (1993) Proc. Natl. Acad. Sci. USA 90:6145-6149; Seto, E. et al. (1993) Nature 365:462-464), the transcriptional cofactor p300 (Lee, J. -S. et al. (1995) Genes & Dev. :1188-1198) and with bZIP-containing proteins such as CREB [(Zhou, Q. et al. (1995) J. Virol. 69:4323-4330), Galvin, K. and Shi, Y., unpublished result)]. The zinc fingers of Sp1 have been shown to interact with the GATA factors (Lee, J. -S. et al. (1995) Genes & Dev. :1188-1198).

The finding that the zinc fingers of WT1 mediate its interaction with par-4 is reminiscent of the WT1/p53 interaction reported previously (Maheswaran, S. et al. (1993) Proc. Natl. Acad. Sci. USA 90:5100-5104). In these studies, the zinc fingers of WT1 were shown to interact with p53, and this p53/WT1 interaction plays a role in WT1-mediated transcriptional repression (Maheswaran, S. et al. (1993) Proc. Natl. Acad. Sci. USA 90:5100-5104). WT1 was shown to inhibit the ability of p53 to induce apoptosis (Maheswaren, S. et al. (1995) Genes & Dev. 9:2143-2156). It thus appears that WT1 may interact with a number of cellular proteins via its zinc finger domain. The biochemical relationship of the three proteins, WT1, p53 and par-4 is currently unknown. It is possible that p53 and par-4 may compete for binding to WT1 since they both bind the zinc fingers of WT1. Alternatively, they may bind to the same WT1 molecule simultaneously. Under certain conditions, p53 and par-4 may be able to bind the same WT1 molecule.

Previously, the transcriptional repression and activation domains of WT1 have been mapped to regions outside the zinc finger domain. A transcriptional cofactor of WT1 is expected to interact with these defined transcriptional domains. Since the WT1/par-4 interaction does not occur within the established transcriptional domains of WT1, it seems that par-4 may not function as a classical co-factor for WT1-mediated transcription. Rather, par-4 may serve a critical modulatory role, i.e., its interaction with WT1 may contribute to the determination of whether WT1 functions as an activator or repressor. Significantly, this presumed modulatory role of par-4 was supported by the observation that overexpression of par-4 overcame growth suppression induced by WT1 (FIG. 10). In this regard, it is interesting to mention the zinc finger-containing transcription factors GATA1 and GATA2, which are believed to play an important role in the development of erythroid cells, megakaryocytes and mast cells. In cell culture, the zinc fingers of GATA-1 alone can rescue GATA-l-deficient embryonic stem cells (Blobel, G. A. et al. (1995) Mol. Cell. Biol. 15:626-633). The C-terminal zinc fingers of these GATA factors are also sufficient to induce megakaryocytic differentiation without the requirement of any known transcriptional domains (Visvader, J. E. et al. (1995) Mol. Cell. Biol. 15:634-641).

The domain within par-4 that mediates its interaction with WT1 has also been identified. The C-terminal leucine repeats of par-4 are necessary and sufficient to mediate the WT1/par-4 physical interaction (FIG. 4B). Thus, the par-4/WT1 interaction occurs via the leucine zipper domain of par-4 and the zinc-finger region of WT1. It has been recently shown that the interaction between the ATF/CREB family of bZIP transcription factors and the zinc-finger protein YY1 is also mediated by leucine zipper/zinc finger interactions (Zhou, Q. et al. (1995) J. Virol. 69:4323-4330). The leucine repeat structure has been found in many transcription factors including Jun, Fos and C/EBP (reviewed in (Johnson, P. F. et al. (1989) Annu. Rev. Biochem. 58:799-839). In addition to the leucine repeats, these transcription factors contain an adjacent subdomain composed of a consensus sequence of predominantly basic amino acids known as the basic region (Pu, W. T. et al. (1991) Mol. Cell. Biol. 11:4918-4926). The leucine repeats are essential for the formation of homo- and hetero-dimeric complexes while the basic region is responsible for the DNA-binding properties of these proteins (Johnson, P. F. et al. (1989) Annu. Rev. Biochem. 58:799-839). As expected, par-4 is capable of homo-oligomerizing and the C-terminal 56 amino acid fragment containing the leucine repeats is necessary and sufficient for oligomerization. However, upon inspection of the par-4 sequence, a consensus basic region is not obvious. While par-4 does not seem to contain an immediate adjacent basic domain, there are stretches of basic residues farther 5′ to the first leucine.

By Northern analysis, par-4 was found to be ubiquitously expressed. Consequently, par-4 and WT1 mRNA were found to be expressed in some of the same adult tissues (FIG. 5). Although circumstantial, this result is consistent with the possibility that the WT1/par-4 interaction is physiologically important. Indeed, physical interactions between the two proteins can be detected in mouse mesonephric cells, and the biological significance of the WT1/par-4 interaction is underscored by the finding that par-4, but not a mutant defective for binding WT1, partially rescued growth suppression caused by WT1.

### EXAMPLE 11

WT1 and par-4 tissue and cellular expression

WT1 has limited expression in adult tissues. Previously, WT1 mRNA was shown to be present in adult human, rat and mouse kidney, ovary, testis, heart, diaphragm, peritoneum and uterus (Armstrong, J. F. et al. (1992) Mec. Dev. 40:85-97; Mundlos, S. et al. (1993) Development 119:1329-1341; Pritchard-Jones, K., et al. (1990) Nature 346:194-197; Rauscher, F. J. (1993) FASEB J. 7:896-903; Sharma, P. M. et al. (1992) Cancer Res. 52:6407-6412). Therefore, adult human polyA⁺ mRNA from a variety of tissues was analyzed for WT1 expression by Northern blot analysis.

A 3.6 kb WT1 mRNA species was strongly expressed in testis and ovary, weakly expressed in heart and kidney (FIG. 5). Marginal expression of WT1 was also found in prostate and colon (FIG. 5). The presence of two transcripts in testis is similar to that observed in mice by Pelletier et al (Pelletier, J. et al. (1991) Genes Dev. 5:1345-1356). The blots were stripped and reprobed with radiolabeled human par-4 cDNA. Three major species designated 1, 2 and 3 of approximately 7.3, 5.0 and 2.1 kb respectively were detected (FIG. 5). The 2.1 kb species is expressed in all tissues, with very weak expression in skeletal muscle and strong expression in testis and ovary. The 5.0 kb species was not detected in brain, skeletal muscle, spleen and peripheral blood lymphocytes, while the 7.3 kb species was not present in brain, skeletal muscle, spleen, thymus and prostate. In another experiment, par-4 mRNA species designated 1, 2 and 3 were also detected by par-4 cDNA probes consisting only of the most 5′ or the most 3′ sequences. As a control for the amount of RNA present on the blot, the blot was reprobed with radiolabeled human actin cDNA (FIG. 5, bottom panel). The three RNA species detected by the par-4 probe represent alternatively spliced isoforms, differentially processed nuclear precursors or closely related family members of par-4. In summary, these data show that par-4 transcripts are ubiquitously expressed.

The subcellular localization of par-4 was next determined. FLAG-par-4 and WT1 were expressed in 293 cells and cells were lysed, fractionated into cytoplasmic and nuclear preparations and probed with α-FLAG and α-WT1 antibodies. As shown in FIG. 6A, while WT1 was found exclusively in the nuclear fraction (lanes 3 and 4), FLAG-par-4 was detected in both the cytoplasmic and nuclear fractions with greater than 50% of par-4 found in the nuclear fraction (FIG. 6A, lanes 1 and 2). As a further control for reliability of the fractionation procedure, a cytoplasmic protein, p₇₀^{s6k} (Reinhard, C. et al. (1994) EMBO Jo. 13:1557-1565), was analyzed and detected only in the cytoplasmic fraction (FIG. 6A, lanes 5 and 6), suggesting that there was minimal cross-contamination. The distribution of the native par-4 protein in the mouse mesonephric cells M15 in which WT1 is expressed (Larsson, S. H. et al. (1995) Cell 81:391-401) was also determined. As shown in FIG. 6B, par-4 was again found in both the nuclear and the cytoplasmic fractions, using affinity-purified α-par-4 polyclonal antibodies (Santa Cruz Biotechnology, Santa Cruz, Calif.). Immunofluorescence analysis of M15 cells with the affinity-purified α-par-4 polyclonal antibodies detected par-4 in the nucleus as well as diffuse staining in the cytoplasm (data not shown).

Taken together, both the fractionation/Western blotting experiments and the immunofluorescence staining identified par-⁴ in both the nucleus and the cytoplasm, consistent with its role as a transcriptional regulator.

### EXAMPLE 12

Par-4 inhibits WT1-mediated transcriptional activation

To determine the functional consequence of the WT1/par-4 interaction, transfection experiments were carried out to analyze the effect of par-4 on both the transcriptional activation and repression activities of WT1. WT1 A and B isoforms have previously been shown to activate a reporter construct containing three WT1/ERG1 binding sites (pEGR₃TKCAT, (Reddy, J. C. et al. (1995) J. Biol. Chem. 270: 10878-10884).

As shown in FIG. 7A, co-transfected WT1 activated this reporter approximately 12-fold in 293 cells (lane 2), similar to the results reported previously (Reddy, J. C. et al. (1995) J. Biol. Chem. 270: 10878-10884). While addition of the pCMV vector had no effect on WT-1-induced activation of the pEGR₃TKCAT reporter (FIG. 7A, lanes 10 and 11), increasing amounts of pCMVpar-4 resulted in a dose dependent decrease in WT1-mediated transcriptional activation (FIG. 7A, lanes 6-9). Par-4 was found not to affect the basal activity of the pEGR₃TKCAT reporter (FIG. 7A, lane 4). The specificity of the WT1/par-4 interaction was demonstrated by examining the effect of par-4 on transcription mediated by EGR1, which shares with WT1 common DNA recognition sites (Rauscher III, F. et al. (1990) Science 250:1259-1263). As shown in FIG. 7B, EGR1 activated the same reporter, pEGR₃TKCAT, in 293 cells (lane 2). However, co-transfection of pCMVpar-4 or pCMV had little effect on EGR1-induced transcriptional activation (FIG. 7B, lanes 4-7).

To rule out the possibility that the observed inhibition of the WT1-induced transcriptional activation by par-4 is due to the effect of par-4 on the expression of genes carried on the co-transfected WT1 plasmid, the levels of WT1 in the presence and absence of transfected par-4 were compared. As shown in FIG. 7C, transfection of 1-15 mg of pCMVpar-4 resulted in an increase in par-4, as judged by Western blotting using α-par-4 antibodies. However, the increase in the expression of transfected par-4 had little effect on the expression level of transfected WT1 protein (FIG. 7C, lanes 1-6). Thus, the decrease in WT-1-mediated transcriptional activation caused by par-4 is not due to a reduction in the WT1 protein level.

To determine whether the physical interaction between WT1 and par-4 underlies their functional interaction, the ability of a mutant par-4 to modulate the transcriptional activity of WT1 was examined. This mutant (pCMVpar-4₁₋₂₆₇) lacks the leucine repeat domain and is unable to physically interact with WT1 (FIG. 4B). As shown in FIG. 7D, par-4₁₋₂₆₇ had little effect on WT-1-mediated transcriptional activation of the pEGR₃TKCAT reporter (lane 8). Thus, the ability of par-4 to inhibit WT-1-mediated transcriptional activation is correlated with its ability to physically interact with WT1. Taken together, these results show that par-4 specifically inhibited WT1-induced transcriptional activation through its physical association with WT1.

### EXAMPLE 13

Par-4 augments WT1-mediated transcriptional repression.

WT1 is capable of activating and repressing transcription. When fused to the DNA-binding domain of GAL4, GAL4-WT1 efficiently repressed the target plasmid pGAL4TKCAT (FIG. 8, lane 2), as reported (Lee, J. -S. et al. (1995) Genes & Dev. :1188-1198). Co-transfection of CMVpar-4 resulted in further repression of the reporter (FIG. 8, lane 3). Since the pCMV vector caused a slight reduction of the CAT activity (FIG. 8, lane 4), the net contribution of par-4 to the enhanced WT1-mediated repression was calculated to be approximately 3-fold. This effect of par-4 on the repression function of WT1 was specific as par-4 did not augment the ability of GAL4-YY1 to repress transcription (FIG. 8, lanes 5-7).

These results indicate that, in addition to inhibiting the activation function of WT1, par-4 specifically enhances its repressor activity.

### EXAMPLE 14

Par-4 is a novel transcriptional repressor.

The fact that par-4 not only inhibited the activation but also enhanced the repression function of WT1 shows that par-4 itself is a transcriptional repressor. To test this model further, full-length par-4 was fused to the GAL4 DNA-binding domain and the fusion protein (GAL4-par-4) was analyzed for transcriptional activity. As shown in FIG. 9, while the GAL4 DNA-binding domain alone had no effect, GAL4-par-4 repressed the CAT expression directed by the GAL4-TKCAT reporter plasmid in a dose-responsive manner (lanes 5-9). This repression was dependent on the GAL4 sites as GAL4-par-4 had no effect on pTKCAT that lacks the GAL4 binding sites (FIG. 9, lane 11).

These results demonstrate that par-4 is capable of repressing transcription when brought to a promoter via a heterologous DNA-binding domain.

### EXAMPLE 15

Par-4 partially rescues WT1-induced growth suppression in a human melanoma cell line.

To determine the biological consequences of the WT1/par-4 interaction, the ability of WT1 and par-4, either alone or together, to regulate cell growth was analyzed in cells of A375-C6, a melanoma cell line (Endo, Y. et al. (1988) J. Immunol 141:2342-2348). The cells were transfected separately with CMV vector, CMV-WT1, CMV-par-4, pCMVpar-41-267, or cotransfected with CMV-WT1 plus CMV-par-4 or pCMVpar-4₁₋₂₆₇.

The number of G418-resistant colonies obtained with the CMV-WT1 plasmid was about 40% of that seen with control vector DNA, with CMV-par-4 or pCMVpar-4₁₋₂₆₇ alone, indicating that WT1 caused growth suppression of the melanoma cells. Cotransfection with CMV-par-4, but not with pCMVpar-4₁₋₂₆₇, restored the colony number to about 90% of that seen with the control vector, suggesting that par-4 may be capable of rescuing cells from growth suppression by WT1, and that the WT1-interacting domain of par-4 was necessary for this effect.

The biological interaction between WT1 and par-4 in A375-C6 cells was also studied by [³H]thymidine incorporation. Results from two different transfectant cell lines (L1 and L2) expressing the indicated plasmids are shown in FIG. 10. Cells transfected with vector DNA, CMV-par-4, or pCMVpar-4₁₋₂₆₇ alone in 72 h of culture showed similar proliferation potential. Consistent with the results from the colony assays, cells transfected with CMV-WT1 showed [³H]thymidine incorporation to be approximately 40% compared with cells transfected with the control vector DNA, deomonstrating growth suppression by WT1. In contrast, cells transfected both with WT1 and par-4 showed about 75% thymidine incorporation compared with vector DNA alone. This represents about 35% increase in thymidine intake compared with cells transfected with WT1 alone, indicating that par-4 partially rescued growth suppression caused by WT1.

The par-4 mutant (CMVpar-4₁₋₂₆₇) that lacks the WT1-interacting domain had no effect on the ability of WT1 to suppress cell growth. Taken together, these results show that WT1 inhibited growth of A375-C6 cells and that the ability of par-4 to overcome growth suppression by WT1 is dependent on the physical interaction between the two proteins.

Several conclusions Are made from the data in Examples 10-15: 1) par-4 is a WT1-interacting protein that modulates the transcriptional activities of WT1 via physical interactions; 2) par-4 overcomes growth suppression caused by WT1 in melanoma cells, possibly as a result of the ability of par-4 to modulate the transcriptional activity of WT1; 3) par-4 is a novel transcriptional repressor. That WT1 and par-4 interact physically is shown by multiple independent protein/protein interaction assays that demonstrated the WT1/par-4 association both in vitro (GST assay) and in vivo (in each of M15 cells, 293 cells, and yeast cells). The significance of the WT1/par-4 interaction is substantiated by the finding that par-4 regulates transcription as well as growth suppression functions of WT1, in a manner that is dependent on the WT1-interacting domain of par-4 (FIG. 7, 8 and 10). Transcriptionally, par-4 specifically inhibited WT1-mediated transcriptional activation but enhanced the ability of WT1 to repress transcription (FIG. 7 and 8). When analyzed as a GAL4-fusion protein, par-4 potently repressed transcription (FIG. 9). These results show that the novel repressor par-4 physically interacts with WT1, bringing in an additional repression domain to the promoter. As a result, par-4 inhibits WT1 activation but potentiates WT1 repression. One biological consequence of this interaction is that par-4 rescues cells whose growth is suppressed by WT1 (FIG. 10).

The mechanisms that control the activation and repression functions of WT1 have yet to be fully elucidated. The number as well as the position of the WT1 binding sites with respect to the TATA box may affect the ability of WT1 to either activate or repress transcription of the reporter plasmids (Wang, Z. -Y. et al. (1993) J. Bio. Chem. 268:9972-9975). The presence or absence of functionally active p53 may also affect the transcriptional activity of WT1 (Maheswaran, S. et al. (1993) Proc. Natl. Acad. Sci. USA 90:5100-5104). We demonstrated in this report that par-4 can modulate the transcriptional activities of WT1 via physical interactions. Using a reporter containing WT1 binding sites that has been shown previously to be specifically activated by WT1 (Reddy, J. C. et al. (1995) J. Biol. Chem. 270: 10878-10884), overexpression of par-4 resulted in a specific dose-dependent decrease in WT1-mediated activation (FIG. 7A). This effect of par-4 was likely to be mediated by its physical interaction with WT1 as the mutant par-4 lacking the WT1-interacting domain failed to inhibit WT-1-induced transcription. Importantly, the effect of par-4 was specific to WT1 since transcription induced by EGR-1, which recognizes the same DNA site as WT1, was unaffected by overexpression of par-4 (FIG. 7B). Furthermore, par-4 was shown to augment the transcriptional repression directed by GAL4-WT1 (FIG. 8).

The mechanism by which par-4 inhibits activation mediated by WT1 can be due to par-4 interfering with the ability of WT1 to bind its recognition sequences. Addition of either in vitro translated or bacterially-produced and purified par-4 protein had no effect on the DNA-binding ability of WT1 in electrophoretic mobility shift assays. An alternative mechanism by which par-4 can inhibit WT1 transcriptional activation is par-4 repressor activity per se. By bringing an additional repressor domain to the promoter via protein-protein interaction with WT1, par-4 can counteract transcriptional activation by WT1. The par-4 protein augments the repression function of WT1, as is shown by assay as a GAL4 fusion protein, in which form par-4 was a potent transcriptional repressor (FIG. 9). These data show that par-4 is a repressor.

That par-4 partially rescued growth suppression of melanoma cells caused by WT1 strengthens the importance of the WT1/par-4 interaction. Transcriptionally, par-4 inhibits activation and also potentiates repression functions of WT1. Further, both activation and repression functions of WT1 have been implicated in its tumor suppressor functions (Haber, D. A. et al. (1993) Science 262:2057-2059; Park, S. et al. (1993) Cancer Res. 53:4757-4760; Reddy, J. C. et al. (1995) J. Biol. Chem. 270: 10878-10884).

In addition to the WT1 gene on chromosome 11p13, WT2 on 11p15.5 has also been implicated in Wilms tumorigenesis (Reeve, A. E. et al. (1989) Mol. Cell. Biol. 9:1799-1803). Chromosomal events causing deletion of chromosome 16 or duplication of chromosome 12 have been correlated with Wilms tumor (Austruy, E. et al. (1995) Genes, Chro. & Cancer. 14:285-294). Other genetic loci have also been suggested to contribute to the disease (Altura, R. A. et al. (1996) Cancer Res. 56:3837-3841). These findings indicate involvement of novel gene products. Mutation or abnormal expression of proteins such as par-4, which modulates both the transcriptional and growth regulatory functions of WT1, could lead also to aberrant expression of certain growth-regulatory proteins, and thus contribute to Wilms tumor formation.

### EXAMPLE 16

par-4 nuclear translocation and role in apovtosis.

As shown in Example 11, par-4 protein can be detected in both the nuclei and the cytoplasmic fractions in Western blotting experiments. Immunofluorescence staining of human 293 cells showed predominant cytoplasmic staining of par-4. Since par-4 functions as a transcriptional regulator, we investigated the conditions under which par-4 may be translocated into the nucleus.

Par-4 has recently been implicated in apoptosis or in sensitizing cells to apoptotic signals. Therefore, we decided first to examine par-4 cellular localization using apoptotic-inducing reagents. Human 293 cells, but not 293 cells transformed with the SV40 large T antigen (293T), have been shown to undergo apoptosis upon treatment with TNF-α and cyclohexamide (16-24 hours post treatment). When 293 and 293T cells were treated with TNF-a and cyclohexamide, nuclear translocation of par-4 was observed after 8-12 hours post in 293, but not 293T cells, using immunofluorescence microscopy. These data show correlations among par-4, its translocation to the nucleus, and induction of the process of apoptosis. Further, treatment of cells with each of TNF-α, cyclohexamide, or actinomycin D also caused nuclear translocation of par-4 after 9 hours. par-4 subcellular localization is thus regulated by the signaling pathways that determine entry into apoptosis.

### EXAMPLE 17

Isolation of proteins that interact with par-4.

To understand the mechanisms underlying par-4 function, we performed the yeast two hybrid assay, using par-4 as a “bait” to identify interacting proteins of par-4. We screened 5×10⁵ colonies of a mouse embryonic library, and identified 6 positive clones which passed the plasmid dependency tests, and were thus considered as clones of potential interacting proteins of par-4.

TABLE 4
Summary of the potential interators identified by the two-hybrid screen

| | | Growth on HIS- | | | | |
|---|---|---|---|---|---|---|
| | Size of | media 25mM 3-AT | | X-gal Assay | | |
| Name of | insert | | pPC97 | | pPC97 | |
| clone | bp | pPC97 | par-4 | pPC97 | par-4 | Sequence |
| 1 A3-3 | 600 | — | + | — | + | Annexin V |
| | | | | | | binding |
| | | | | | | protein |
| 1 A4-4 | 600 | — | + | — | + | Annexin V |
| | | | | | | binding |
| | | | | | | protein |
| 1 A5-5 | 600 | — | + | — | + | Annexin V |
| | | | | | | binding |
| | | | | | | protein |
| 1 B4-2 | 600 | — | + | — | + | Annexin V |
| | | | | | | binding |
| | | | | | | protein |
| 1 B7-3 | 600 | — | + | — | + | Annexin V |
| | | | | | | binding |
| | | | | | | protein |
| 9 A2-1 | 1800 | — | + | — | ++ | ERK 5 |

As shown in Table 4, five out of the six clones were identical, and encode a mouse protein that is highly homologous to human annexin V binding protein. Annexin V is a protein that binds to lipoproteins in the cell membrane (Dubois T, et al (1996) Biochim Biophys Acta, Vol. 1313, 290-294). It has also been shown to inhibit the kinase activity of conventional isoforms of protein kinase C.

Another clone encodes a sequence that is the mouse homolog of human ERK5 (extracellar related signal-regulated kinase) kinase. ERK5 is a member of the MAP kinase family, proteins that are components of protein kinase cascades that respond to extracellular stimuli, and comprise the signal transduction pathway. ERK5 is distinct from other ERKs in that it contains an extra C-terminal domain. (Abe, J. et al. (1996) J. Biol. Chemistry, 271, 16586-16590; Zhou, G. et al. (1995) J. Biol. Chemistry, 270, 12665-12669). Thus par-4 can function in the cytoplasm to modulate activities of kinases that play a role in apoptosis and other signal transduction pathways, can bind to annexin V binding proteins and modulate cytoskeletal events, and also bind to WT1 and modulate its transcriptional regulatory activity.

It is to be understood that while the invention has been described in conjunction with the detailed description thereof, that the foregoing description is intended to illustrate and not limit the scope of the invention, which is defined by the scope of the appended claims. Other aspects, advantages, and modifications within the scope of the invention will be apparent to those skilled in the art to which the invention pertains.

All patents and publications referred to herein are hereby incorporated by reference.

What is claimed is:

1. A protein comprising the amino acid sequence of SEQ ID No: 3.

2. An isolated ciao-1 protein or a portion thereof that is a WT1 binding protein.

3. An isolated protein comprising an amino acid sequence at least 75% homologous to the amino acid sequence of SEQ ID No: 3, wherein the protein is a WT1 binding protein.

4. The isolated protein of claim 3 comprising an amino acid sequence at least 85% homologous to the amino acid sequence of SEQ ID No: 3.

5. The isolated protein of claim 4 comprising an amino acid sequence at least 95% homologous to the amino acid sequence of SEQ ID No: 3.

6. The protein of claim 3 that is a naturally occurring mutant or polymorphism of the protein of SEQ ID No: 3 or a portion thereof that is a WT1 binding protein.

7. An isolated or recombinant protein encoded by a nucleic acid which hybridizes under stringent conditions to a nucleic acid sequence represented in SEQ ID No: 1.

8. A human par-4 protein or a portion thereof that is a WT1 binding protein.

9. A protein comprising the amino acid sequence of SEQ ID No: 4.

10. An isolated protein comprising an amino acid sequence at least 95% identical to the amino acid sequence shown in SEQ ID No: 4, wherein the protein is a WT1 binding protein.

11. The isolated protein of claim 10 comprising an amino acid sequence at least 99% identical to the amino acid sequence shown in SEQ ID No: 4.

12. An isolated or recombinant human par-4 protein encoded by a nucleic acid which hybridizes under stringent conditions to a nucleic acid sequence represented in SEQ ID No: 2.

13. The protein of claim 10 that is a naturally occurring mutant or polymorphism of the protein of SEQ ID No.: 4., or a portion thereof that is a WT1 binding protein.

14. A par-4 deletion mutant wherein the par-4 protein comprises the leucine zipper motif of residues 305-340 shown in SEQ ID No: 4.

15. An antibody that specifically binds a ciao-1 protein.

16. An antibody that specifically binds a human par-4 protein.

17. A ciao-1 fusion protein.

18. A human par-4 fusion protein.

19. The fusion protein of claim 17 or claim 18, wherein said fusion protein is functional in a two-hybrid assay.

20. An isolated nucleic acid molecule comprising a nucleotide sequence encoding ciao-1 protein.

21. An isolated nucleic acid molecule comprising a nucleotide sequence encoding a protein, wherein the protein: (i) comprises an amino acid sequence at least 75% homologous to the amino acid sequence of SEQ ID No.: 3 and (ii) is a binding protein of WT1 protein.

22. The isolated nucleic acid molecule of claim 21, wherein the protein comprises an amino acid sequence at least 85% homologous to the amino acid sequence of SEQ ID No.: 3.

23. The isolated nucleic acid molecule of claim 21, wherein the protein comprises an amino acid sequence at least 95% homologous to the amino acid sequence of SEQ ID No.: 3.

24. An isolated nucleic acid molecule comprising a nucleotide sequence encoding achuman par-4 protein.

25. An isolated nucleic acid molecule comprising a nucleotide sequence encoding a protein, wherein the protein: (i) comprises an amino acid sequence at least 95% homologous to the amino acid sequence of SEQ ID No: 4 and (ii) is a binding protein of WT1 protein.

26. The isolated nucleic acid molecule of claim 3, wherein the protein comprises an amino acid sequence at least 99% homologous to the amino acid sequence of SEQ ID No.: 4.

27. A vector comprising the nucleic acid molecule of claim 20.

28. The vector of claim 27, which is a recombinant expression vector.

29. A host cell containing the vector of claim 28.

30. A method for producing a ciao-1 protein comprising culturing the host cell of claim 44 in a suitable medium until a ciao-1 protein is produced.

31. The method of claim 30, further comprising isolating the ciao-1 protein from the host cell or the medium.

32. A vector comprising the nucleic acid molecule of claim 24.

33. The vector of claim 32, which is a recombinant expression vector.

34. A host cell containing the vector of claim 33.

35. A method for producing a human par-4 protein comprising culturing the host cell of claim 34 in a suitable medium until the human par-4 protein is produced.

36. The method of claim 35, further comprising isolating the human par-4 protein from the host cell or the medium.

37. A nonhuman transgenic animal having cells carrying a transgene encoding a ciao-1 protein or a portion of ciao-1 protein that is a WT1 binding protein.

38. The nonhuman transgenic animal of claim 37, wherein the transgene alters an endogenous gene encoding an endogenous ciao-1 protein.

39. A nonhuman transgenic animal having cells carrying a transgene encoding human par-4 protein or a portion of human par-4 protein that is a WT1 binding protein.

40. The nonhuman transgenic animal of claim 39, wherein the transgene alters an endogenous gene encoding an endogenous par-4 protein.

41. A method for detecting the presence of ciao-1 activity in a biological sample, comprising contacting the biological sample with an agent capable of detecting an indicator of ciao-1 activity, such that the presence of ciao-1 is detected in the biological sample.

42. The method of claim 41, wherein the agent detects ciao-1 mRNA.

43. The method of claim 42, wherein the agent is a labeled nucleic acid probe capable of hybridizing to ciao-1 mRNA.

44. The method of claim 41, wherein the agent detects ciao-1 protein.

45. The method of claim 44, wherein the agent is a labeled antibody that specifically binds to ciao-1 protein.

46. A method for detecting the presence of human par-4 activity in a biological sample, comprising contacting the biological sample with an agent capable of detecting an indicator of human par-4 activity, such that the presence of human par-4 is detected in the biological sample.

47. The method of claim 46, wherein the agent detects human par-4 mRNA.

48. The method of claim 47, wherein the agent is a labeled nucleic acid probe capable of hybridizing to human par-4 mRNA.

49. The method of claim 46, wherein the agent detects human par-4 protein.

50. The method of claim 49, wherein the agent is a labeled antibody that specifically binds to human par-4 protein.

51. A method for identifying an agent that modulates the binding of a ciao-1 protein to WT1, comprising:
combining:
(i) a WT1 protein or a ciao-1 binding portion thereof;
(ii) and a ciao-1 protein or the WT-1-binding portion thereof,
in the presence and absence of the agent; and determining the degree of binding of (i) and (ii) in the presence and absence of the agent,
wherein a statistically significant change in the degree of binding in the presence of the agent identifies an agent that modulates the binding of the ciao- I protein to the WT1 protein.

52. A method for identifying an agent that modulates the binding of a par-4 protein to WT1, comprising:
combining:
(i) a WT1 protein or a par-4 binding portion thereof;
(ii) and a par-4 protein or the WT1-binding portion thereof,
in the presence and absence of the agent; and
determining the degree of binding of (i) and (ii) in the presence and absence of the agent,
wherein a statistically significant change in the degree of binding in the presence of the agent identifies an agent that modulates the binding of the par-4 protein to the WT1 protein.

53. A method for identifying an agent that modulates the binding of a par-4 protein to a par-4 binding protein, comprising:
combining:
(i) a par-4 protein or a binding portion thereof specific for an ERK5 protein or an annexin V binding protein;
(ii) and a par-4 binding protein which is an ERK5 protein or an annexin V protein or the par-4 binding portion thereof,
in the presence and absence of the agent; and
determining the degree of binding of (i) and (ii) in the presence and absence of the agent,
wherein a statistically significant change in the degree of binding in the presence of the agent identifies an agent that modulates the binding of the par-4 protein to the par-4 binding protein.


SEQUENCE LISTING (7 sequences)

SEQ ID NO: 1 - DNA (cDNA), 1413 bp; both-stranded, linear
  CDS 157..1176
ggtgagtttccggggcgacccaggtcctccggcggaacggggagcctctgtcggccgcggaagcctggagtgggcggtacgcagacgcgcgcggcgagacccgctgtctgctcagcggactctgcccgcccccacctccccctgcgtcgggccgacatgaaggactcgctggtgctgctgggccgtgtcccggcgcacccggactcccgttgctggttcctggcctggaaccccgcggggaccctgctggcctcgtgcggcggcgaccggagaatccgcatctggggcacggagggtgacagctggatctgcaagtctgtcctttctgaaggccaccagcgcaccgtgcggaaggtagcctggtccccctgcggtaattacctggcctctgccagctttgatgctaccacttgcatttggaagaagaaccaggatgactttgagtgtgtaaccactctcgagggccatgaaaatgaggtcaagtcagtggcttgggccccatctggcaacctcctggccacttgcagccgagataagagcgtttgggtctgggaagttgatgaagaggatgagtatgaatgtgtcagtgttctcaactcccacacacaggatgtcaagcatgtggtttggcacccaagtcaggagctcttagcttctgccagctatgatgacacagtgaagctgtaccgggaggaagaggatgactgggtatgctgtgccacccttgagggccatgaatccactgtgtggagcttggcctttgacccgagtggccagcgcctggcgtcttgtagtgatgaccgtactgtgcgtatctggcgtcagtatctaccaggcaatgaacaaggggtggcatgcagcggctctgaccccagttggaaatgtatctgtactttgtccggcttccactcaaggaccatttatgacattgcttggtgtcagctgacaggggctctggccacagcttgtggggatgacgcgatccgcgtgtttcaggaggatcccaactcggatccacagcagcccaccttctccctgacagcccacttgcatcaggcccattcccaggatgtcaactgtgtggcctggaaccccaaggagccagggctactggcctcctgcagtgatgatggggaggtggccttctggaagtatcagcggcctgaaggcctctgagctaccacgtctttggacagagtaatgactccccagaaaacgtcatataagacttttaccagcccctgagaggaccaggaggagcatccttgaccttcatttaacttggctcacttctcttcagacttgggtagaagtgcagagccacagaattgctttccttccccgcctttgacatgaggccttcagtaaagagctacagaacatgaaaaaaaaaaaaaaaaaaaaaaaaaaaaa

SEQ ID NO: 2 - DNA (cDNA), 1719 bp; both-stranded, linear
  CDS 239..1267
ctttccgcgcggcggaagagcgcgcgccagcttcggcacacttgggagccggatcccagccctacgcctcgtcccctacaagctcctccaagccccgccggctgctgtgggagcggcggccgtcctctcctggaggtcgtctcctggcatcctcggggccgcaggaaggaagaggaggcagcggccggagccctggtgggcggcctgaggtgagagcccgaccggcccctttgggaatatggcgaccggtggctaccggaccagcagcggcctcggcggcagcaccacagacttcctggaggagtggaaggcgaaacgcgagaagatgcgcgccaagcagaaccccccgggcccggcccccccgggagggggcagcagcgacgccgctgggaagccccccgcgggggctctgggcaccccggcggccgccgctgccaacgagctcaacaacaacctcccgggcggcgcgccggccgcacctgccgtccccggtcccgggggcgtgaactgcgcggtcggctccgccatgctgacgcgggcgcccccggcccgcggcccgcggcggtcggaggacgagcccccagccgcctctgcctcggctgcaccgccgccccagcgtgacgaggaggagccggacggcgtcccagagaagggcaagagctcgggccccagtgccaggaaaggcaaggggcagatcgagaagaggaagctgcgggagaagcggcgctccaccggcgtggtcaacatccctgccgcagagtgcttagatgagtacgaagatgatgaagcagggcagaaagagcggaaacgagaagatgcaattacacaacagaacactattcagaatgaagctgtaaacttactagatccaggcagttcctatctgctacaggagccacctagaacagtttcaggcagatataaaagcacaaccagtgtctctgaagaagatgtctcaagtagatattctcgaacagatagaagtgggttccctagatataacagggatgcaaatgtttcaggtactctggtttcaagtagcacactggaaaagaaaattgaagatcttgaaaaggaagtagtaacagaaagacaagaaaacctaagacttgtgagactgatgcaagataaagaggaaatgattggaaaactcaaagaagaaattgatttattaaatagagacctagatgacatagaagatgaaaatgaacagctaaagcaggaaaataaaactcttttgaaagttgtgggtcagctgaccaggtagaggattcaagactcaatgtggaaaaaatattttaaactactgattgaatgttaatggtcaatgctagcacaatattcctatgctgcaatacattaaaataactaagcaagtatatttatttctagcaaacagatgtttgttttcaaaatacttctttttcattattggttttaaaaaagcattatccttttatctcacaaataagtaatatctttcagttattaaatgatagataatgcctttttggttttgtgtggtattcaactaatacatggtttaaagtcacagccgtttgaatatattttatcttggtagtacattttctcccttaggaatatacatagtctttgtttacatgagttccaatacttttgggatgttaccctcacatgtccctatactgatgtgtgccaccttttatgtgttgatgactcactcataaggttttggtc

SEQ ID NO: 3 - protein, 339 aa
MKDSLVLLGRVPAHPDSRCWFLAWNPAGTLLASCGGDRRIRIWGTEGDSWICKSVLSEGHQRTVRKVAWSPCGNYLASASFDATTCIWKKNQDDFECVTTLEGHENEVKSVAWAPSGNLLATCSRDKSVWVWEVDEEDEYECVSVLNSHTQDVKHVVWHPSQELLASASYDDTVKLYREEEDDWVCCATLEGHESTVWSLAFDPSGQRLASCSDDRTVRIWRQYLPGNEQGVACSGSDPSWKCICTLSGFHSRTIYDIAWCQLTGALATACGDDAIRVFQEDPNSDPQQPTFSLTAHLHQAHSQDVNCVAWNPKEPGLLASCSDDGEVAFWKYQRPEGL

SEQ ID NO: 4 - protein, 342 aa
MATGGYRTSSGLGGSTTDFLEEWKAKREKMRAKQNPPGPAPPGGGSSDAAGKPPAGALGTPAAAAANELNNNLPGGAPAAPAVPGPGGVNCAVGSAMLTRAPPARGPRRSEDEPPAASASAAPPPQRDEEEPDGVPEKGKSSGPSARKGKGQIEKRKLREKRRSTGVVNIPAAECLDEYEDDEAGQKERKREDAITQQNTIQNEAVNLLDPGSSYLLQEPPRTVSGRYKSTTSVSEEDVSSRYSRTDRSGFPRYNRDANVSGTLVSSSTLEKKIEDLEKEVVTERQENLRLVRLMQDKEEMIGKLKEEIDLLNRDLDDIEDENEQLKQENKTLLKVVGQLTR

SEQ ID NO: 5 - DNA (cDNA), 15 bp; single-stranded, linear
gaattctgcggccgc

SEQ ID NO: 6 - DNA (cDNA), 24 bp; single-stranded, linear
taccagcctcttgctgagtggaga

SEQ ID NO: 7 - DNA (cDNA), 24 bp; single-stranded, linear
tagacaagccgacaaccttgattg